(19) United States
(12) Patent Application Publication
(10) Pub. No.: US 2020/0063166 A1
(43) Pub. Date: Feb. 27, 2020

(54) ZINC BINUCLEAR CLUSTER TRANSCRIPTIONAL REGULATOR-DEFICIENT STRAIN

(71) Applicant: DSM IP Assets B.V., Heerlen (NL)

(72) Inventors: Noeel Nicolaas Maria Elisabeth VAN PEIJ, Echt (NL); Peter Jozef Ida VAN DE VONDERVOORT, Echt (NL)

(21) Appl. No.: 16/493,017

(22) PCT Filed: Mar. 12, 2018
(86) PCT No.: PCT/EP2018/056015

§ 371 Date: Sep. 11, 2019

(30) Foreign Application Priority Data
Mar. 13, 2017 (EP) 17160536.3

(51) Int. Cl. C12P 1/02; C07K 14/38; C12N 15/80

(52) U.S. Cl. CPC C12P 1/02; C12N 15/80; C07K 14/38

(57) ABSTRACT

The present disclosure relates to a mutant filamentous fungal host cell which is deficient in the zinc binuclear cluster transcriptional regulator oreR or in a functional homologue thereof if compared with a parent filamentous fungal host cell which has not been modified and measured under the same conditions. It has been surprisingly found that when the mutant filamentous fungal host cell according to the disclosure is used in a method to produce a compound of interest by microbial fermentation, for example an enzyme, substantially no oxalic acid is produced extracellularly by the cell during the fermentation as a by-product, which allows a more economical and efficient recovery of the compound of interest from the fermentation broth.

### FIELD

The disclosure relates to a mutant filamentous fungal host cell which has been modified to result in a deficiency in the production of a zinc binuclear cluster transcriptional regulator or the DNA binding domain thereof, to a method to produce the mutant filamentous fungal host cell and to a method to produce a compound of interest using said mutant filamentous host cell.

### BACKGROUND

Filamentous fungal host cells are widely used for the industrial production of useful compounds such as polypeptides, e.g. enzymes, and secondary metabolites. Prior to their use as production hosts, filamentous fungi are often modified to reduce the secretion of enzymes and/or other substances which can interfere with the expression, recovery, and purification of said useful compounds.

Oxalic acid is a fungal secondary metabolite and by-product in filamentous fungal fermentations, for example in Aspergillus niger. The occurrence of oxalic acid in the fermentation broth is not desired because of its toxicity and the formation of insoluble salts with calcium ions. Such insoluble oxalate salts hamper the downstream processing of industrial fermentations and may lead to a lower product yield and to damages to the downstream processing equipment. Furthermore, part of the carbon source used in the fermentation broth is wasted in the production of oxalic acid instead of being used to produce the desired product. The latter renders the fermentation process less economical.

Several solutions have been proposed to reduce or eliminate the presence of oxalic acid in filamentous fungal fermentations. Pedersen and Christensen (Metabolic Engineering (2000) 2: 34-41) and WO00/50576 described Aspergillus niger strains wherein the gene oahA coding for the enzyme oxaloacetate hydrolase, was disrupted. Oxaloacetate hydrolase catalyses the hydrolysis in the cell of oxaloacetate into oxalic acid and acetate and a deficiency in this enzyme yields a cell with a reduced production of oxalic acid. WO2004/070022 A2 described oxalate-deficient A. niger strains capable of expressing a polypeptide of interest which were produced by UV-irradiation and selection of low oxalate producing strains which at the same time had no impairment in polypeptide expression.

Despite above solutions there is still a need for new oxalate-deficient filamentous fungal cells which can be used for the expression of useful compounds in an industrial setting.

Zinc-binding proteins form one of the largest families of transcriptional regulators in eukaryotes. Within this group, Class III (C₆) zinc finger proteins contain a DNA binding domain which comprises six cysteine residues bound to two zinc atoms and they are named zinc cluster, zinc binuclear cluster or Zn(II)₂Cys₆ (Zn₂C₆) proteins (MacPherson et al, Microbiol. Mol. Biol. Rev. (2006) 70(3): 583-604). The Saccharomyces cerevisiae Gal4 transcription factor is the most studied zinc binuclear cluster in this family. In yeast members of the Gal4 transcription factor superfamily are involved in many cellular processes including sugar metabolism, gluconeogenesis, respiration, amino acid metabolism etcetera. Zn(II)₂Cys₆ transcriptional factors are also found abundantly in filamentous fungi (even though functionally much less characterised then in yeast) wherein some have been found to be involved e.g. in the regulation of the expression of extracellular enzymes for biomass degradation, metabolism and catabolism, secondary metabolites such as aflatoxin or fumonisin for example (Knox and Keller in Zeilinger et al. (Eds.) Biosynthesis and Molecular Genetics of Fungal Secondary Metabolites, vol. 2, chapter 2, Springer Science+Business Media New York 2015, ISBN: 978-1-4939-2530-8).

The analysis of Aspergillus niger genome has revealed the presence of more than 300 transcriptional regulators belonging to the Zn(II)₂Cys₆ family (Pel et al Nature Biotechnology (2007) 25(2): 221-231, Supplementary Table 4, http://www.nature.com/nbt/journal/v25/n2/extref/nbt1282-S21.pdf). The function of many of these factors remains until now largely unknown. Very recently a study aimed at the identification of Carbohydrate-Active enZymes (CAZy) genes relevant to lignocellulose degradation has shed some light on the role of some transcriptional regulators in the expression of CAZymes. In this study, it has been postulated that one of these factors, An14g5670, might play a role in the activation of pectinases which are induced in Aspergillus during lignocellulose degradation (Daly et al. Biotechnol. Biofuels (2017) 10:35, DOI 10.1186/s13068-017-0700-9).

### SUMMARY

The disclosure provides in a first aspect a mutant filamentous fungal host cell which has been modified to result in a deficiency in the cell of a polypeptide comprising a polypeptide selected from:
a. a polypeptide with an amino acid sequence according to SEQ ID NO: 5 or a polypeptide with an amino acid sequence at least 70% identical to the amino acid sequence according to SEQ ID NO: 5 which is a functional equivalent of the polypeptide with the amino acid sequence according to SEQ ID NO: 5;
b. a polypeptide with an amino acid sequence according to SEQ ID NO: 6 or a polypeptide with an amino acid sequence at least 70% identical to the amino acid sequence according to SEQ ID NO: 6 which is a functional equivalent of the polypeptide with the amino acid sequence according to SEQ ID NO: 6;
c. a polypeptide with an amino acid sequence according to SEQ ID NO: 4 or a polypeptide with an amino acid sequence at least 70% identical to the amino acid sequence according to SEQ ID NO: 4 which is a functional equivalent of the polypeptide with the amino acid sequence according to SEQ ID NO: 4;
d. a polypeptide encoded by a polynucleotide with a nucleotide sequence according to SEQ ID NO: 2 or 3 or encoded by a polynucleotide at least 70% identical to a nucleotide sequence according to SEQ ID NO: 2 or 3, wherein said polypeptide encoded by a polynucleotide at least 70% identical to a nucleotide sequence according to SEQ ID NO: 2 or 3 is a functional equivalent of the polypeptide encoded by the polynucleotide according to SEQ ID NO: 2 or 3;
e. a polypeptide encoded by a polynucleotide capable of hybridising under low, medium or high stringency conditions to the complementary strand of a polynucleotide with a nucleotide sequence according to SEQ ID NO: 2 or 3 or a polypeptide encoded by a polynucleotide capable of hybridizing under low, medium or high stringency conditions to the complementary strand of a polynucleotide with a nucleotide sequence at least 70% identical to a nucleotide sequence according to SEQ ID NO: 2 or 3, wherein said polypeptide encoded by a polynucleotide capable of hybridizing under low, medium or high stringency conditions to the complementary strand of a polynucleotide at least 70% identical to a nucleotide sequence according to SEQ ID NO: 2 or 3 is a functional equivalent of the polypeptide encoded by the polynucleotide according to SEQ ID NO: 2 or 3;

if compared with a parent filamentous fungal host cell which has not been modified when measured under the same conditions.

The disclosure further provides a method of producing a mutant filamentous fungal host cell according to the first aspect of the disclosure comprising the steps of:
a) providing a parent filamentous fungal host cell;
b) modifying the parent filamentous fungal host cell to yield a mutant filamentous fungal host cell which is deficient in the cell in a polypeptide comprising a polypeptide selected from:
   i. a polypeptide with an amino acid sequence according to SEQ ID NO: 5 or a polypeptide with an amino acid sequence at least 70% identical to the amino acid sequence according to SEQ ID NO: 5 which is a functional equivalent of the polypeptide with the amino acid sequence according to SEQ ID NO: 5;
   ii. a polypeptide with an amino acid sequence according to SEQ ID NO: 6 or a polypeptide with an amino acid sequence at least 70% identical to the amino acid sequence according to SEQ ID NO: 6 which is a functional equivalent of the polypeptide with the amino acid sequence according to SEQ ID NO: 6;
   iii. a polypeptide with an amino acid sequence according to SEQ ID NO: 4 or a polypeptide with an amino acid sequence at least 70% identical to the amino acid sequence according to SEQ ID NO: 4 which is a functional equivalent of the polypeptide with the amino acid sequence according to SEQ ID NO: 4;
   iv. a polypeptide encoded by a polynucleotide with a nucleotide sequence according to SEQ ID NO: 2 or 3 or encoded by a polynucleotide at least 70% identical to a nucleotide sequence according to SEQ ID NO: 2 or 3, wherein said polypeptide encoded by a polynucleotide at least 70% identical to a nucleotide sequence according to SEQ ID NO: 2 or 3 is a functional equivalent of the polypeptide encoded by the polynucleotide with the nucleotide sequence according to SEQ ID NO: 2 or 3;
   v. a polypeptide encoded by a polynucleotide capable of hybridising under low, medium or high stringency conditions to the complementary strand of a polynucleotide with a nucleotide sequence according to SEQ ID NO: 2 or 3 or a polypeptide encoded by a polynucleotide capable of hybridizing under low, medium or high stringency conditions to the complementary strand of a polynucleotide with a nucleotide sequence at least 70% identical to a nucleotide sequence according to SEQ ID NO: 2 or 3, wherein said polypeptide encoded by a polynucleotide capable of hybridizing under low, medium or high stringency conditions to the complementary strand of a polynucleotide at least 70% identical to a nucleotide sequence according to SEQ ID NO: 2 or 3 is a functional equivalent of the polypeptide encoded by the polynucleotide with the nucleotide sequence according to SEQ ID NO: 2 or 3;
if compared with a parent filamentous fungal host cell which has not been modified when measured under the same conditions.

In a third aspect, the disclosure provides a method for the production of a compound of interest by microbial fermentation comprising:
a) providing a mutant filamentous fungal host cell according to the first aspect of the disclosure or obtained by a method according to the second aspect of the disclosure, capable of expressing the compound of interest,
b) culturing said mutant filamentous fungal host cell under conditions conducive to the expression of the compound of interest,
c) optionally isolating the compound of interest from the culture medium.

### DESCRIPTION OF THE FIGURES

FIG. 1 depicts a schematic representation of the oreR disruption strategy employed.

FIG. 2a to FIG. 2e depict the alignment of a) the genomic DNA of An14g05670 gene, with introns indicated in italic, b) the oreR cDNA and c) the resulting sequence remaining in the genome after disruption (indicated as “disruption sequence”).

FIG. 3 depicts the phylogenetic identification of OreR (An14g05670, indicated as XP_001401190.2 in the figure)-orthologous transcription factors with the OreA transcription factor protein marked with a grey box. The Neighbor-Joining tree of the OreR homologs was assembled using the BLOSUM 62 matrix with assembly of alignment using Neighbor—Joining phylogeny as available in the Clone Manager Professional, version 9 for Windows (© 2016 Scientific & Educational Software). The orthologues in the phylogenetic tree are as follows: XP_001401190.2 is OreR, EHA27958.1 is hypothetical protein ASPNIDRAFT_41900 (Aspergillus niger ATCC 1015), CAK42128.1 is an unnamed protein product (Aspergillus niger), GAA90677.1 is a C6 finger domain protein (Aspergillus kawachii IFO 4308), GAT19234.1 is a C6 finger domain protein (Aspergillus luchuensis), OJZ84680.1 is hypothetical protein ASPFODRAFT_62587 (Aspergillus luchuensis CBS 106.47), OJ189400.1 is hypothetical protein ASPTUDRAFT_60162 (Aspergillus tubingensis CBS 134.48), OJJ77274.1 is hypothetical protein ASPBRDRAFT_115071 (Aspergillus brasiliensis CBS 101740), XP_002850264.1 is a C6 zinc finger domain-containing protein (Arthroderma otae CBS 113480), CEJ54143.1 is a Putative C6 zinc finger domain protein (Penicillium brasilianum), KJK66719.1 is a Zn2-Cys6 binuclear cluster domain protein (Aspergillus parasiticus SU-1), XP_002380179.1 is a putative C6 finger domain protein (Aspergillus flavus NRRL3357), XP_001818758.2 is a C6 finger domain protein (Aspergillus oryzae RIB40), XP_015405413.1 is a putative C6 finger domain protein (Aspergillus nomius NRRL 13137), OGM46820.1 is a C6 finger domain protein (Aspergillus bombycis), CRL26276.1 is an Aflatoxin biosynthesis regulatory protein (Penicillium camemberti), XP_002563980.1 is Pc20g15060 (Penicillium rubens Wisconsin 54-1255), CEL10827.1 is a Putative C6 zinc finger domain protein (Aspergillus calidoustus), KKK23905.1 is hypothetical protein AOCH_004198 (Aspergillus ochraceoroseus), XP_020057899.1 is hypothetical protein ASPACDRAFT_77277 (Aspergillus aculeatus ATCC 16872), OOF93142.1 is hypothetical protein ASPCADRAFT_209754 (Aspergillus carbonarius ITEM 5010) (All proteins mentioned herein are available in the sequence databases accessible for instance via the National Centrum for Biotechnology Information (NCBI https://www.ncbi.nlm.nih.gov/), EMBL (http://www.ebi.ac.uk/embl/) or DNA Database of Japan (DDBJ—http://www.ddbj.nig.ac.jp/).

FIG. 4 depicts the alignment of most closely related oreR orthologues to the OreR reference sequence (An14g05670/XP_001401190.2), including the deduction of a consensus sequence. The orthologues in the alignments are as follows: XP_001401190.2 is OreR, GAT19234.1 is a C6 finger domain protein (Aspergillus luchuensis), OJ189400.1 is hypothetical protein ASPTUDRAFT_60162 (Aspergillus tubingensis CBS 134.48), OJJ77274.1 is hypothetical protein ASPBRDRAFT_115071 (Aspergillus brasiliensis CBS 101740), OJZ84680.1 is hypothetical protein ASPFODRAFT_62587 (Aspergillus luchuensis CBS 106.47), CAK42128.1 is an unnamed protein product (Aspergillus niger), EHA27958.1 is hypothetical protein ASPNIDRAFT_41900 (Aspergillus niger ATCC 1015).

FIG. 5 depicts the alignment of GAL4-like Zn(II)2Cys6 (or C6 zinc) or zinc binuclear cluster DNA-binding domain for several zinc binuclear transcription factors orthologues closely related to OreR (An14g05670/XP_001401190.2). Underlined and in bold is the Zn²⁺ binding domain with the 6 cysteines involved in zinc binding. From this alignment, the DNA binding domain with a consensus sequence

DLRDRHRRRCikXXgqerXSKrKSCXXCAQKKIRCsXtRpXCXRCXQXXXXCXYP can be deduced (wherein X may be any natural amino acid). The orthologues in the alignment are the following: XP_001401190.2 is OreR, GAT19234.1 is a C6 finger domain protein (Aspergillus luchuensis), KJK66719.1 is a Zn2-Cys6 binuclear cluster domain protein (Aspergillus parasiticus SU-1), KKK23905.1 is hypothetical protein AOCH_004198 (Aspergillus ochraceoroseus), OGM46820.1 is a C6 finger domain protein (Aspergillus bombycis), OJ189400.1 is hypothetical protein ASPTUDRAFT_60162 (Aspergillus tubingensis CBS 134.48), OJJ77274.1 is hypothetical protein ASPBRDRAFT_115071 (Aspergillus brasiliensis CBS 101740), OJZ84680.1 is hypothetical protein ASPFODRAFT_62587 (Aspergillus luchuensis CBS 106.47), OOF93142.1 is hypothetical protein ASPCADRAFT_209754 (Aspergillus carbonarius ITEM 5010), XP_001818758.2 is a C6 finger domain protein (Aspergillus oryzae RIB40), XP_002380179.1 is a putative C6 finger domain protein (Aspergillus flavus NRRL3357), XP_015405413.1 is a putative C6 finger domain protein (Aspergillus nomius NRRL 13137), CRL26276.1 is an Aflatoxin biosynthesis regulatory protein (Penicillium camemberti), CAK42128.1 is an unnamed protein product (Aspergillus niger), CEJ54143.1 is a Putative C6 zinc finger domain protein (Penicillium brasilianum), XP_002563980.1 is Pc20g15060 (Penicillium rubens Wisconsin 54-1255), XP_020057899.1 is hypothetical protein ASPACDRAFT_77277 (Aspergillus aculeatus ATCC 16872), CEL10827.1 is a Putative C6 zinc finger domain protein (Aspergillus calidoustus), XP_002850264.1 is a C6 zinc finger domain-containing protein (Arthroderma otae CBS 113480).

### DESCRIPTION OF THE SEQUENCE LISTING

SEQ ID NO: 1 depicts the protein sequence of proline specific endoprotease BC2G079 (previously described in WO2015177171, example 1, as SEQ ID NO: 2).

SEQ ID NO: 2 depicts the genomic sequence of the An14g05670 gene (indicated herewith as oreR gene), including 5′ and 3′ sequences from Aspergillus niger.

SEQ ID NO: 3 depicts the coding sequence (cDNA) of the An14g05670 gene (oreR gene) from Aspergillus niger.

SEQ ID NO: 4 depicts the protein sequence of An14g05670 from Aspergillus niger, indicated herewith as OreR (oxalate C6 transcriptional regulator).

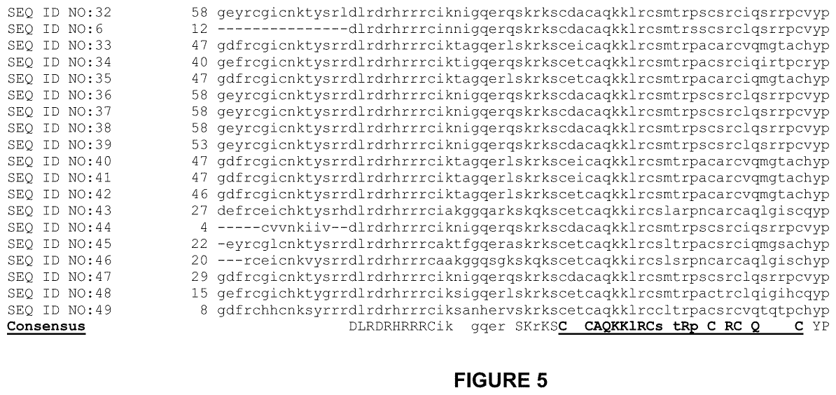

SEQ ID NO: 5 depicts the consensus sequence of the DNA binding domain as deduced from the alignment of the protein sequences as shown in FIG. 5 (wherein X can be any natural amino acid).

SEQ ID NO: 6 depicts the putative DNA binding domain of OreR (XP_001401190.2) from Aspergillus niger.

SEQ ID NO: 7: depicts the putative DNA binding domain of GAT19234.1 from Aspergillus luchuensis.

SEQ ID NO: 8 depicts the putative DNA binding domain of KJK66719.1 from Aspergillus parasiticus SU-1.

SEQ ID NO: 9 depicts the putative DNA binding domain of KKK23905.1 from Aspergillus ochraceoroseus.

SEQ ID NO: 10 depicts the putative DNA binding domain of OGM46820.1 from Aspergillus bombycis.

SEQ ID NO: 11 depicts the putative DNA binding domain of OJ189400.1 from Aspergillus tubingensis CBS 134.48.

SEQ ID NO: 12 depicts the putative DNA binding domain of OJJ77274.1 from Aspergillus brasiliensis CBS 101740.

SEQ ID NO: 13 depicts the putative DNA binding domain of OJZ84680.1 from Aspergillus luchuensis CBS 106.47.

SEQ ID NO: 14 depicts the putative DNA binding domain of OOF93142.1 from Aspergillus carbonarius ITEM 5010.

SEQ ID NO: 15 depicts the putative DNA binding domain of XP_001818758.2 from Aspergillus oryzae RIB40.

SEQ ID NO: 16 depicts the putative DNA binding domain of XP_002380179.1 from Aspergillus flavus NRRL3357.

SEQ ID NO: 17 depicts the putative DNA binding domain of XP_015405413.1 from Aspergillus nomius NRRL 13137.

SEQ ID NO: 18 depicts the putative DNA binding domain of CRL26276.1 from Penicillium camemberti.

SEQ ID NO: 19 depicts the putative DNA binding domain of CAK42128.1 from Aspergillus niger.

SEQ ID NO: 20 depicts the putative DNA binding domain of CEJ54143.1 from Penicillium brasilianum.

SEQ ID NO: 21 depicts the putative DNA binding domain of XP_002563980.1 from Penicillium rubens Wisconsin 54-1255.

SEQ ID NO: 22 depicts the putative DNA binding domain of XP_020057899.1 from Aspergillus aculeatus ATCC 16872.

SEQ ID NO: 23 depicts the putative DNA binding domain of CEL10827.1 from Aspergillus calidoustus.

SEQ ID NO: 24 depicts the putative DNA binding domain of XP_002850264.1 from Arthroderma otae CBS 113480.

SEQ ID NO: 25 depicts the protein sequence of GAT19234.1 from Aspergillus luchuensis.

SEQ ID NO: 26 depicts the protein sequence of OJ189400.1 from Aspergillus tubingensis CBS 134.48.

SEQ ID NO: 27 depicts the proteins sequence of OJJ77274.1 from Aspergillus brasiliensis CBS 101740.

SEQ ID NO: 28 depicts the protein sequence of OJZ84680.1 from Aspergillus luchuensis CBS 106.47.

SEQ ID NO: 29 depicts the protein sequence of CAK42128.1 from Aspergillus niger.

### DETAILED DESCRIPTION

The present inventors have surprisingly found that the gene An14g5670, coding for the putative zinc binuclear transcriptional regulator oreR, might be involved in the regulation of the metabolic pathway and/or secretion pathway in filamentous fungi of oxalic acid and other organic acids sharing the same or related metabolic pathways and/or secretion pathways such as citric acid, itaconic acid, malic acid, fumaric acid or succinic acid, preferably citric acid and/or itaconic acid. It has been surprisingly found that cells deficient in oreR, when used in the fermentative production of a compound of interest, produce considerably less oxalic acid extracellularly as a by-product or have entirely lost the ability to produce extracellularly said acid.

The disclosure therefore provides in a first aspect a mutant filamentous fungal host cell which has been modified to result in a deficiency in the cell of a polypeptide comprising a polypeptide selected from:
a. a polypeptide with an amino acid sequence according to SEQ ID NO: 5 or a polypeptide with an amino acid sequence at least 70% identical to SEQ ID NO: 5 which is a functional equivalent of the polypeptide with the amino acid sequence according to SEQ ID NO: 5;
b. a polypeptide with an amino acid sequence according to SEQ ID NO: 6 or a polypeptide with an amino acid sequence at least 70% identical to SEQ ID NO: 6 which is a functional equivalent of the polypeptide with the amino acid sequence according to SEQ ID NO: 6;
c. a polypeptide with an amino acid sequence according to SEQ ID NO: 4 or a polypeptide with an amino acid sequence at least 70% identical to SEQ ID NO: 4 which is a functional equivalent of the polypeptide with the amino acid sequence according to SEQ ID NO: 4;
d. a polypeptide encoded by a polynucleotide with a nucleotide sequence according to SEQ ID NO: 2 or 3 or encoded by a polynucleotide at least 70% identical to the nucleotide sequence according to SEQ ID NO: 2 or 3, wherein said polypeptide encoded by a polynucleotide at least 70% identical to SEQ ID NO: 2 or 3 is a functional equivalent of the polypeptide encoded by the polynucleotide with the nucleotide sequence according to SEQ ID NO: 2 or 3;
e. a polypeptide encoded by a polynucleotide capable of hybridising under low, medium or high stringency conditions to the complementary strand of a polynucleotide with a nucleotide sequence according to SEQ ID NO: 2 or 3 or a polypeptide encoded by a polynucleotide capable of hybridizing under low, medium or high stringency conditions to the complementary strand of a polynucleotide with a nucleotide sequence at least 70% identical to the nucleotide sequence according to SEQ ID NO: 2 or 3, wherein said polypeptide encoded by a polynucleotide capable of hybridizing under low, medium or high stringency conditions to the complementary strand of a polynucleotide at least 70% identical to the nucleotide sequence according to SEQ ID NO: 2 or 3 is a functional equivalent of the polypeptide encoded by the polynucleotide with the nucleotide sequence according to SEQ ID NO: 2 or 3;

if compared with a parent filamentous fungal host cell which has not been modified when measured under the same conditions, preferably wherein the percentage of sequence identity in a. to e. is measured over the full length of the amino acid sequences according to a. to e.

A polypeptide with amino acid sequence according to SEQ ID NO: 6 is the putative DNA binding domain of the OreR polypeptide encoded by gene An14g05670 (also indicated as XP_001401190.2). An14g05670 is indicated herewith as OreR, i.e. oxalate C6 (or zinc binuclear) transcriptional regulator. The polypeptide sequence of OreR is the amino acid sequence according to SEQ ID NO: 4 and it is encoded by the polynucleotide with a nucleotide sequence according to SEQ ID NO: 2 (genomic DNA) and SEQ ID NO: 3 (cDNA).

A polypeptide with amino acid sequence according to SEQ ID NO: 5 is the consensus sequence for the putative DNA binding domain of a zinc binuclear cluster transcriptional regulator obtained from the alignment of the oreR sequence and its closest orthologue sequences, as described in example 3.

Preferably a polypeptide according to the first aspect in a. or b. comprises or is a DNA binding domain of a zinc binuclear cluster transcriptional regulator or it is able to perform the function of a DNA binding domain of a zinc binuclear cluster transcriptional regulator or it is a functional fragment thereof.

Preferably a polypeptide according to the first aspect in c. to e. comprises or is a zinc binuclear cluster transcriptional regulator or it is able to perform the function of a zinc binuclear cluster transcriptional regulator or it is a functional fragment thereof.

The term “transcriptional regulator (indicated as well as transcriptional factor or transcription regulator or transcription factor)” has herewith the same meaning as it is given in in the pertinent art (see e.g. Latchman D. S. Int. J. Biochem. Cell Biol. (1997) 29(12): 1305-1312; Alberts B, Johnson A, Lewis J, et al. Molecular Biology of the Cell. 4th edition. New York: Garland Science; 2002. ISBN-10: 0-8153-3218-1ISBN-10: 0-8153-4072-9, From DNA to RNA. Available from: https://www.ncbi.nlm.nih.gov/books/NBK26887/). A transcriptional regulator is defined in the art as a protein that controls the rate of transcription of a gene from DNA to messenger RNA, by binding to a specific DNA sequence. A transcriptional regulator comprises therefore a DNA binding domain, i.e. the portion of the transcriptional regulator which is able to bind DNA. The term “DNA binding domain” is herewith given the same meaning as generally accepted in the pertinent art. A DNA binding domain is defined in the art as an independently folded domain which contains a structural motif able to recognize and bind DNA (Alberts B, Johnson A, Lewis J, et al. Molecular Biology of the Cell. 4th edition. New York: Garland Science; 2002. DNA-Binding Motifs in Gene Regulatory Proteins. Available from: https://www.ncbi.nlm.nih.gov/books/NBK26806/).

A “zinc binuclear cluster (or zinc cluster or Zn(II)₂Cys₆ or Zn₂C₆) transcriptional regulator” is a Class III (C₆) zinc finger proteins transcriptional regulator containing a DNA binding domain which comprises 6 cysteine residues bound to two zinc atoms (MacPherson et al, Microbiol. Mol. Biol. Rev. (2006) 70(3): 583-604), with a highly conserved, consensus Cys-rich amino acid sequence according to sequence Cys-X₂-Cys-X₆-Cys-X₅₋₁₂-Cys-X₂-Cys-X₆₋₈-Cys, commonly located at the N-terminus. The Saccharomyces cerevisiae Gal4 transcriptional regulator is the most studied transcriptional regulator in this group.

Preferably the mutant filamentous fungal host cell according to the first aspect of the disclosure produces and/or secretes less oxalic acid than the parent filamentous fungal host cell which has not been modified, when cultured and measured under the same conditions. Preferably the mutant filamentous fungal host cell according to the first aspect of the disclosure produces extracellularly less oxalic acid than the parent filamentous fungal host cell which has not been modified, when cultured and measured under the same conditions. Preferably the mutant filamentous fungal host cell according to the first aspect of the disclosure has lost the ability to produce extracellularly oxalic acid if compared with the parent filamentous fungal host cell which has not been modified, when cultured and measured under the same conditions.

In one preferred embodiment, the mutant filamentous fungal host cell according to the first aspect of the disclosure produces and/or secretes less oxalic acid, or produce extracellularly less oxalic acid than the parent filamentous fungal host cell which has not been modified, when cultured and measured under the same conditions, wherein oxalic acid is determined using methods known to those skilled in the art. For example, the assay for oxalate detection as described in WO2004/070022 (Examples, page 18, Assay for oxalate detection in A. niger culture supernatant) can be used or the method described in “Dionex Corporation, (now sold under Thermo Scientific Brand Thermo Scientific) “Determination of Inorganic Anions and Organic Acids in Fermentation Broths”. Application Note 123; Sunnyvale, Calif., USA). Preferably an assay as herein described in the examples is used. In a preferred embodiment the filamentous fungal host cell according to the disclosure produces extracellularly less oxalic acid than the parent filamentous fungal host cell which has not been modified, when cultured and measured under the same conditions or has lost the ability to produce oxalic acid extracellularly if compared with the parent filamentous fungal host cell which has not been modified, when cultured and measured under the same conditions.

In one embodiment, the mutant filamentous fungal host cell according to the first aspect of the disclosure produces and/or secretes less oxalic acid, or produces extracellularly less oxalic acid, than the parent filamentous fungal host cell which has not been modified, when cultured under the same conditions and when measured under the same conditions, preferably wherein the measurement is performed according to the assay comprising the steps of: pre-treating a fermentation broth supernatant by mixing 10 parts of the supernatant and 1 part of a 3.3 N HClO₄, solution, keeping in refrigerator for 1 hour, centrifuging, diluting the obtained supernatant up to an (expected) concentration of oxalic acid in the sample between 0.1-30 g/l yielding a pre-treated sample, injecting 20 micro liters of pre-treated sample on a Phenomenex® Rezex™ RHM-Monosaccharide column with diameter 7.8 mm and length of 300 mm, with a column temperature of 50° C., eluting a flow of 0.6 ml/min during 27 minutes, using 0.01 N H₂SO₄ as eluent, and UV detection at 214 nm. The Phenomenex® Rezex™ RHM-Monosaccharide column is an ion-exclusion column comprising 8% cross-linked sulfonated styrene divinyl benzene resin in the hydrogen ionic form. It will be clear to the skilled person that in case the Phenomenex® Rezex™ RHM-Monosaccharide would not be available anymore it may be replaced in the above assay with an analogous column as suggested by the manufacturer and under analogous conditions as suggested by the manufacturer (Phenomenex-USA).

In another embodiment, the mutant filamentous fungal host cell may produce and/or secrete less or more, itaconic acid, citric acid, malic acid if compared with the parent filamentous fungal host cell which has not been modified, when cultured and measured under the same conditions.

The disclosure relates in one aspect to a zinc binuclear cluster transcriptional regulator which is preferably involved in the extracellular production of oxalic acid by a filamentous fungal host cell. The latter is evidenced by the fact that a filamentous fungal host cells which is deficient in the zinc binuclear cluster transcriptional regulator according to the disclosure produces extracellularly less oxalic acid than the parent cell which has not been modified, when cultured and measured under the same conditions, or said mutant cell has lost the ability to extracellularly produce oxalic acid if compared with the parent cell which has not been modified, when cultured and measured under the same conditions. Said zinc binuclear cluster transcriptional regulator may be able to regulate the expression of one or more genes involved in the production or degradation of oxalic acid inside the cell. Alternatively said zinc binuclear cluster transcriptional regulator may be able to regulate the expression of one or more genes involved in the active or passive transport of oxalic acid through the membrane and/or cell wall of the filamentous fungal host cell. The resulting response can include, but is not limited to the up- or downregulation of one or several acid transport or export proteins, decrease or increase in expression of genes encoding enzymes or proteins involved in production or degradation of oxalic acid, respectively. Enzymes known and described to be involved in oxalate metabolism or catabolism are for example oxalate oxidase (EC 1.2.3.4), lactate dehydrogenase (EC 1.1.1.27), glycolate oxidase (EC 1.1.3.15), oxalate decarboxylase (EC 4.1.1.2), oxaloacetate (acetyl)hydrolase (EC 3.7.1.1), oxalyl CoA synthase (EC 6.2.1.8), oxalate oxidoreductase (EC 1.2.7.10) or oxalate CoA transferase (EC 2.8.3.2). The disclosure also relates to the DNA binding domains of said zinc binuclear cluster transcriptional regulators. In one embodiment of the first aspect of the disclosure the polypeptide, preferably comprising or beings a DNA binding domain of a zinc binuclear cluster transcriptional regulator or being able to perform the function of a DNA binding domain of a zinc binuclear cluster transcriptional regulator, or a functional fragment thereof, comprises a) a polypeptide with an amino acid sequence according to SEQ ID NO: 5 or b) a polypeptide with an amino acid sequence at least 50% identical, at least 60%, at least 70%, at least 75%, preferably at least 80% identical to the amino acid sequence according to SEQ ID NO: 5 and which is a functional equivalent of the polypeptide with the amino acid sequence according to SEQ ID NO: 5. In a further embodiment the polypeptide has an amino acid sequence which is at least 85% identical, or at least 90% identical, or at least 91%, 92%, 93%, 94%, 95%, 96%, 97%, 98% or at least 99% identical to the amino acid sequence according to SEQ ID NO: 5 and is a functional equivalent of the polypeptide with the amino acid sequence according to SEQ ID NO: 5. Preferably sequence identity is measured over the full length of the amino acid sequence of the polypeptide with at least 50% sequence identity to the amino acid sequence according to SEQ ID NO: 5.

A “functionally equivalent polypeptide” (or functional equivalent) is herewith defined as a polypeptide which retains the qualitative biological function or activity of the polypeptide to which it is compared, when measured under the same conditions. For example, it may display the same or similar (enzymatic) activity when measured under the same conditions, or binds the same DNA recognition site and/or regulate the same repertoire of genes. It is noted that a functionally equivalent polypeptide may have an altered quantitative biological function or activity in respect with the polypeptide to which it is compared.

A “functional fragment” refers herewith to a portion or sub-sequence of a parent biological sequence, e.g. a polypeptide that retains the qualitative biological function or activity of the parent polypeptide, when measured under the same conditions. For example, it may display the same or similar (enzymatic) activity when measured under the same conditions, or binds the same DNA recognition site and/or regulate the same repertoire of genes. It is noted that a functional fragment polypeptide may have an altered quantitative biological function or activity in respect with the parent polypeptide.

In another embodiment, the polypeptide comprising a polypeptide with an amino acid sequence according to SEQ ID NO: 5 or the polypeptide comprising a polypeptide with an amino acid sequence which is at least 50% identical to the amino acid sequence according to SEQ ID NO: 5 and is functionally equivalent to the polypeptide with the amino acid sequence according to SEQ ID NO: 5 will be able to function as or to perform the activity of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell. The polypeptide comprising the polypeptide with an amino acid sequence which is at least 50% identical to the amino acid sequence according to SEQ ID NO: 5 may be more or less active or have the same activity as the polypeptide with an amino acid sequence according to SEQ ID NO: 5 when measured under the same conditions.

In another embodiment of the first aspect of the disclosure the polypeptide, preferably comprising or being a DNA binding domain of a zinc binuclear cluster transcriptional regulator or being able to perform the function of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell, or a functional fragment thereof, comprises a polypeptide with an amino acid sequence according to SEQ ID NO: 6 or a polypeptide with an amino acid sequence at least 50% identical, at least 60%, at least 70%, at least 75%, preferably at least 80% identical to the amino acid sequence according to SEQ ID NO: 6 and which is a functional equivalent of the polypeptide with the amino acid sequence according to SEQ ID NO: 6. In a further embodiment the polypeptide has an amino acid sequence which is at least 85% identical, or at least 90% identical, or at least 91%, 92%, 93%, 94%, 95%, 96%, 97%, 98% or at least 99% identical to the amino acid sequence according to SEQ ID NO: 6 and is a functional equivalent of the polypeptide with the amino acid sequence according to SEQ ID NO: 6. Preferably sequence identity is measured over the full length of the amino acid sequence of the polypeptide with at least 50% sequence identity to the amino acid sequence according to SEQ ID NO: 6.

In one embodiment, the polypeptide comprising a polypeptide with an amino acid sequence according to SEQ ID NO: 6 or comprising the polypeptide with an amino acid sequence which is at least 50% identical to the amino acid sequence according to SEQ ID NO: 6 and is functionally equivalent to the polypeptide with an amino acid sequence according to SEQ ID NO: 6 will be able to function as DNA binding domain of a zinc binuclear cluster transcriptional regulator, preferably a DNA binding domain of a zinc binuclear cluster transcriptional regulator involved in the extracellular production of oxalic acid by a filamentous fungal host cell. The polypeptide comprising the polypeptide with an amino acid sequence which is at least 50% identical to the amino acid sequence according to SEQ ID NO: 6 may be more or less active or have the same activity as the polypeptide comprising a polypeptide with an amino acid sequence according to SEQ ID NO: 6 when measured under the same conditions.

In yet another embodiment of the first aspect of the disclosure the polypeptide, preferably comprising or being a zinc binuclear cluster transcriptional regulator or being able to perform the function of a zinc binuclear cluster transcriptional regulator, preferably of a zinc binuclear cluster transcriptional regulator involved in the extracellular production of oxalic acid by a filamentous fungal host cell, or a functional fragment thereof, is a polypeptide comprising a polypeptide with an amino acid sequence according to SEQ ID NO: 4 or a polypeptide comprising a polypeptide with an amino acid sequence at least 50% identical, at least 60%, preferably at least 70% identical or at least 75% identical to the amino acid sequence according to SEQ ID NO: 4 which is a functional equivalent of the polypeptide with the amino acid sequence according to SEQ ID NO: 4. In a further embodiment the polypeptide comprises a polypeptide with an amino acid sequence which is at least 80% identical, at least 85% identical, preferably at least 90% identical or at least 91%, 92%, 93%, 94%, 95%, 96%, 97%, 98% or at least 99% identical to the amino acid sequence according to SEQ ID NO: 4 and is a functional equivalent of the polypeptide with the amino acid sequence according to SEQ ID NO: 4. Preferably sequence identity is measured over the full length of the amino acid sequence of the polypeptide with at least 50% sequence identity to the amino acid sequence according to SEQ ID NO: 4.

In one embodiment, the polypeptide comprising a polypeptide with an amino acid sequence according to SEQ ID NO: 4 or the polypeptide comprising a polypeptide with an amino acid sequence which is at least 50% identical to the amino acid sequence according to SEQ ID NO: 4 and is a functional equivalent of the polypeptide with the amino acid sequence according to SEQ ID NO: 4 will be able to function as a zinc binuclear cluster transcriptional regulator, preferably a zinc binuclear cluster transcriptional regulator involved in the extracellular production of oxalic acid by a filamentous fungal host cell. The polypeptide comprising a polypeptide with an amino acid sequence which is at least 50% identical to the amino acid sequence according to SEQ ID NO: 4 may be more or less active or have the same activity as the polypeptide comprising a polypeptide with an amino acid sequence according to SEQ ID NO: 4 when measured under the same conditions.

In one embodiment, a polypeptide comprising a polypeptide with an amino acid sequence which is at least 50% identical to the amino acid sequence according to SEQ ID NO: 4 and is functionally equivalent to the polypeptide with an amino acid sequence according to SEQ ID NO: 4 may be a polypeptide comprising an amino acid sequence according to any one of SEQ ID NO: 25, 26, 27, 28, or 29.

In one further embodiment of the first aspect of the disclosure the polypeptide, preferably comprising or being a zinc binuclear cluster transcriptional regulator or being able to perform the function of a zinc binuclear cluster transcriptional regulator, preferably of a zinc binuclear cluster transcriptional regulator involved in the extracellular production of oxalic acid in a filamentous fungal cell, or a functional fragment thereof, is encoded by a polynucleotide with a nucleotide sequence according to SEQ ID NO: 2 or 3 or encoded by a polynucleotide at least 50% identical to the nucleotide sequence according to SEQ ID NO: 2 or 3, wherein said polypeptide encoded by a polynucleotide at least 50% identical to SEQ ID NO: 2 or 3 is a functional equivalent of the polypeptide encoded by the polynucleotide with a nucleotide sequence according to SEQ ID NO: 2 or 3. In one embodiment the polypeptide is encoded by a polynucleotide at least 50% identical, at least 60% identical to a nucleotide sequence according to SEQ ID NO: 2 or 3, at least 70% identical, at least 75% identical, at least 80% identical, at least 85% identical, at least 90% identical to a nucleotide sequence according to SEQ ID NO: 2 or 3, or at least 91% identical, at least 92%, at least 93%, at least 94%, at least 95%, at least 96%, at least 96%, at least 97%, at least 98% or at least 99% % identical to a nucleotide sequence according to SEQ ID NO: 2 or 3, wherein said polypeptide encoded by a polynucleotide at least 50%, at least 60%, at least 80%, at least 90% identical to a nucleotide sequence according to SEQ ID NO: 2 or 3, or at least 91%, 92%, 93%, 94%, 95%, 96%, 97%, 98%, 99% identical to a nucleotide sequence according to SEQ ID NO: 2 or 3 is a functional equivalent of the polypeptide encoded by the polynucleotide according to a nucleotide sequence according to SEQ ID NO: 2 or 3. Preferably sequence identity is measured over the full length of the polynucleotide sequence of the polynucleotide which is at least 50% identical to the polynucleotide with a nucleotide sequence according to SEQ ID NO: 2 or 3.

In still another embodiment according to the first aspect of the disclosure the polypeptide, preferably comprising or being a zinc binuclear cluster transcriptional regulator or being able to perform the function of a zinc binuclear cluster transcriptional regulator, preferably of a zinc binuclear cluster transcriptional regulator involved in the extracellular production of oxalic acid in a filamentous fungal cell, or a functional fragment thereof, is a polypeptide comprising a polypeptide encoded by a polynucleotide capable of hybridising under low stringency, preferably under medium stringency, most preferably under high stringency conditions to the complementary strand of a polynucleotide with a nucleotide sequence according to SEQ ID NO: 2 or 3 or a polypeptide encoded by a polynucleotide capable of hybridizing under low stringency, preferably under medium stringency, most preferably under high stringency conditions to the complementary strand of a polynucleotide with a nucleotide sequence at least 50% identical to SEQ ID NO: 2 or 3, wherein said polypeptide encoded by a polynucleotide capable of hybridizing under low stringency, preferably under medium stringency, most preferably under high stringency conditions conditions to the complementary strand of a polynucleotide at least 50% identical to a nucleotide sequence according to SEQ ID NO: 2 or 3 is a functional equivalent of the polypeptide encoded by the polynucleotide with a nucleotide sequence according to SEQ ID NO: 2 or 3. In one embodiment the polypeptide comprising the polypeptide encoded by a polynucleotide capable of hybridising under low stringency, preferably under medium stringency, most preferably under high stringency conditions to the complementary strand of a polynucleotide with a nucleotide sequence according to SEQ ID NO: 2 or 3 or a polypeptide encoded by a polynucleotide capable of hybridizing under low stringency, preferably under medium stringency, most preferably under high stringency conditions to the complementary strand of a polynucleotide with a nucleotide sequence at least 50% identical, at least 60% identical to a nucleotide sequence according to SEQ ID NO: 2 or 3, preferably at least 70% identical, at least 80% identical, at least 90% identical, at least 91%, 92%, 93%, 94%, 95%, 96%, 97%, 98%, 99% identical to a nucleotide sequence according to SEQ ID NO: 2 or 3, wherein said polypeptide encoded by a polynucleotide capable of hybridizing under low stringency, preferably under medium stringency, most preferably under high stringency conditions to the complementary strand of a polynucleotide at least 50% identical, at least 60% identical to a nucleotide sequence according to SEQ ID NO: 2 or 3, preferably at least 70% identical, at least 80% identical, at least 90% identical, at least 91%, 92%, 93%, 94%, 95%, 96%, 97%, 98%, 99% identical to a nucleotide sequence according to SEQ ID NO: 2 or 3, is a functional equivalent of the polypeptide encoded by the polynucleotide with a nucleotide sequence according to SEQ ID NO: 2 or 3.

Within the context of the present disclosure “measured under the same conditions” or “analysed under the same conditions” means that the mutant filamentous fungal host cell and the parent cell are cultivated under the same conditions and that the amount and/or activity of the polypeptide in which the mutant cell is deficient, compared to the parent cell, is measured for the mutant cell and for the parent cell, respectively, using the same conditions, preferably by using the same assay and/or methodology, more preferably within the same experiment.

For the purpose of this disclosure, to determine the percentage of sequence identity of two amino acid sequences or of two nucleic acid sequences, the sequences are aligned for optimal comparison purposes. To optimize the alignment between the two sequences gaps may be introduced in any of the two sequences that are compared. Such alignment may be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will know that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percentage of sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE.

For the purpose of this disclosure the NEEDLE program from the EMBOSS package is used to determining sequence identity (version 2.8.0 or higher, EMBOSS: The European Molecular Biology Open Software Suite (2000) Rice, P. Longden, I. and Bleasby, A. Trends in Genetics 16, (6) pp. 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the disclosure is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

To determine multiple-sequence alignments, programs such as ClustalW, ClustalX Larkin M A, Blackshields G, Brown N P, Chenna R, McGettigan P A, McWilliam H, Valentin F, Wallace I M, Wilm A, Lopez R, Thompson J D, Gibson T J, Higgins D G (2007). “ClustalW Cand ClustalX version 2”. Bioinformatics. 23 (21): 2947-2948. doi:10.1093/bioinformatics/btm404.) or Clustal Omega (Sievers, Fabian; Higgins, DesmondG. (2014-01-01). Russell, David J, ed. Multiple Sequence Alignment Methods. Methods in Molecular Biology. Humana Press. pp. 105-116. doi:10.1007/978-1-62703-646-7_6. ISBN 9781627036450; Sievers, Fabian; Higgins, Desmond G. (2002-01-01). Current Protocols in Bioinformatics. John Wiley & Sons, Inc. doi:10.1002/0471250953.bi0313s48. ISBN 9780471250951) may be used. The Clustal programs are e.g. available at http://www.clustal.org/or at the EMBL-EBI webserver at http://www.ebi.ac.uk/Tools/msa/). Alternatively, the Clone Manager Professional, (version 9.4 for Windows© 2015 Scientific & Educational Software) may also be used for multiple protein sequence alignment (e.g. using the EBLOSUM62 matrix).

The nucleic acid and protein sequences disclosed herewith can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

As used herein, the term “hybridizing” is intended to describe conditions for hybridization and washing under which polynucleotide sequences at least about 60%, 65%, 80%, 85%, 90%, preferably at least 93%, more preferably at least 95% and most preferably at least 98% identical to each other typically remain hybridized to the complement of each other. As used herein, the term “hybridization” means the pairing of substantially complementary strands of oligomeric compounds. One mechanism of pairing involves hydrogen bonding, which may be Watson-Crick, Hoogsteen or reversed Hoogsteen hydrogen bonding, between complementary nucleotide bases (nucleotides) of the strands of oligomeric compounds. For example, adenine and thymine are complementary nucleic acids which pair through the formation of hydrogen bonds. Hybridization can occur under varying circumstances. “Stringency hybridization” or “hybridizes under low stringency, medium stringency, high stringency, or very high stringency conditions” is used herein to describe conditions for hybridization and washing, more specifically conditions under which an oligomeric compound will hybridize to its target sequence, but to a minimal number of other sequences. So, the oligomeric compound will hybridize to the target sequence to a detectably greater degree than to other sequences. Guidance for performing hybridization reactions can be found in Current Protocols in Molecular Biology, John Wiley & Sons, N.Y. (1989), 6.3.1-6:3.6.

The skilled artisan will know which conditions to apply for low, medium and high stringency hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

Stringency conditions are sequence-dependent and will be different in different circumstances. Longer sequences hybridize specifically at higher temperatures. Generally, stringency conditions are selected to be about 5° C. lower than the thermal melting point (Tₘ) for the oligomeric compound at a defined ionic strength and pH. The Tₘ is the temperature (under defined ionic strength and pH) at which 50% of an oligomeric compound hybridizes to a perfectly matched probe. Stringency conditions may also be achieved with the addition of destabilizing agents such as formamide.

Examples of specific hybridization conditions are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5M sodium phosphate, 7% SDS at 65° C., followed by one or more washes at 0.2×SSC, 1% SDS at 65° C.

In one embodiment, the polypeptide which has an amino acid sequence according to SEQ ID NO: 5 or the polypeptide which has an amino acid sequence having at least 50% identity to an amino acid sequence according to SEQ ID NO: 5 and which is functionally equivalent to an amino acid sequence according to SEQ ID NO: 5, which preferably comprises or is a DNA binding domain of a zinc binuclear cluster transcriptional regulator or is able to perform the function of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell, is a polypeptide which when aligned with the sequence set out in SEQ ID NO: 5, comprises one or more amino acids of

Ala or Ile at position 11, Lys, Asn or Ala at position 12, Gly or Asn at position 15, Gin or His at position 16, Glu, Ser or Ala at position 17, Gly or Arg at position 18, Arg or Gin at position 22, Ile of Leu at position 33, Ser or Cys at position 36, Thr, Ser or Ala at position 38, Pro or Ser at position 40, said positions being defined with reference to SEQ ID NO: 5.

An amino acid position corresponding to one of the positions defined herein in the polypeptide with reference to SEQ ID NO: 5, may be a position in the polypeptide that aligns in a multiple (protein) sequence alignment with any of the stated amino acid positions in the amino acid sequence according to SEQ ID NO: 5.

An amino acid position corresponding to one of the positions indicated above, said position being defined with reference to SEQ ID NO: 5 is a position which is identified in the polypeptide sequence when the latter is aligned with the amino acid sequence set out in SEQ ID NO: 5 by a suitable sequence alignment method. A suitable sequence alignment method is a method which allows comparison of the sequences with each other and identifications of the positions in the amino acid sequence of polypeptide wherein either the same amino acid is present (identical position), or another amino acid is present (substitution), or one or more extra amino acids are present (insertion or extension) or no amino acid is present (deletion or truncation) if compared with the amino acid sequence set out in SEQ ID NO: 5.

A suitable method allowing comparison of two amino acid sequences may be any suitable Pairwise Sequence Alignment method known to those skilled in the art, preferably a Global Pairwise Sequence Alignment method. A preferred Global Pairwise Sequence Alignment method is the EMBOSS Needle method based on the Needleman-Wunsch alignment algorithm (aiming at finding the optimum alignment (including gaps) of the two sequences along their entire length) (Needleman, S. B. and Wunsch, C. D. (1970) J. Mol. Biol. 48, 443-453) as described herein. In one embodiment, the amino acid sequence of the polypeptide is aligned with the amino acid sequence set out in SEQ ID NO: 5 using the EMBOSS Needle alignment method, such as the NEEDLE program from the EMBOSS package, using EBLOSUM62 as a substitution matrix, preferably with a gap-open penalty of 10 and a gap extension penalty of 0.5. In one embodiment, the polypeptide which has an amino acid sequence according to SEQ ID NO: 5 or the polypeptide which has an amino acid sequence having at least 50% identity to an amino acid sequence according to SEQ ID NO: 5 and which is functionally equivalent to an amino acid sequence according to SEQ ID NO: 5, which preferably comprises or is a DNA binding domain of a zinc binuclear cluster transcriptional regulator or is able to perform the function of a DNA binding domain of a zinc binuclear cluster transcriptional regulator, preferably a DNA binding domain of a zinc binuclear cluster transcriptional regulator involved in the production and/or secretion of oxalic acid by a filamentous fungal host cell, is a polypeptide which, when aligned with the sequence set out in SEQ ID NO: 5, comprises one or more of

Ile at position 11, Lys at position 12, Gly at position 15, Gin at position 16, Glu at position 17, Arg at position 18, Arg at position 22, Leu at position 33, Ser at position 36, Thr at position 38, Pro at position 40,

said positions being defined with reference to SEQ ID NO: 5.

In yet another embodiment, the polypeptide with an amino acid sequence according to SEQ ID NO: 5 or the polypeptide which has an amino acid sequence with at least 50% identity to an amino acid sequence according to SEQ ID NO: 5 and which is functionally equivalent to an amino acid sequence according to SEQ ID NO: 5, which preferably comprises or is a DNA binding domain of a zinc binuclear cluster transcriptional regulator or is able to perform the function of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell, is a polypeptide which, when aligned with the sequence set out in SEQ ID NO: 5, comprises one or more of

an amino acid with a polar uncharged side chain at position 13, an amino acid with a hydrophobic side chain at position 14, an amino acid with a negatively charged side chain at position 26, an amino acid with a hydrophobic side chain at position 37, an amino acid with an hydrophobic side chain at position 46,

said positions being defined with reference to SEQ ID NO: 5.

In the context of the present disclosure an amino acid with a polar uncharged side chain is an amino acid selected from Ser, Thr, Asn or Gin. In the context of the present disclosure an amino acid with a negatively charged side chain is an amino acid selected from Asp or Glu. An amino acid with a hydrophobic side chain is herein intended as an amino acid selected from Ala, Val, Ile, Leu, Met, Phe, Tyr or Trp.

In yet another embodiment, the polypeptide with an amino acid sequence according to SEQ ID NO: 5 or the polypeptide which has an amino acid sequence having at least 50% identity to an amino acid sequence according to SEQ ID NO: 5 and which is functionally equivalent to an amino acid sequence according to SEQ ID NO: 5 comprises or is a DNA binding domain of a zinc binuclear cluster transcriptional regulator or is able to perform the function of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell which, when aligned with the sequence set out in SEQ ID NO: 5, comprises one or more of

Ser, Asn or Thr at position 13, Ala, Phe or Ile at position 14, Asp or Glu at position 26, Leu or Met at position 37, Val, Ala, Leu or Ile at position 46,

said positions being defined with reference to SEQ ID NO: 5.

In another embodiment, the polypeptide which has an amino acid sequence according to SEQ ID NO: 5 or the polypeptide which has an amino acid sequence with at least 50% identity to an amino acid sequence according to SEQ ID NO: 5 and which is functionally equivalent to an amino acid sequence according to SEQ ID NO: 5, which preferably is or comprises a DNA binding domain of a zinc binuclear cluster transcriptional regulator or is able to perform the function of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell, is a polypeptide comprising an amino acid sequence according to any one of SEQ ID NO: 6, 7, 8, 9, 10, 11, 12, 13, 14, 15, 16, 17, 18, 19, 20, 21, 22, 23 or 24.

Within the context of the present disclosure the mutant filamentous fungal host cell is deficient in a polypeptide as defined herein when the host cell comprises a modification which results in a reduced presence or production or in no presence or production in the cell of the polypeptide as defined herein if compared to the parent filamentous fungal host cell that has not been modified, when measured under the same conditions and/or comprises a modification which results in a polypeptide with decreased or no activity and/or with reduced or no function, when compared to the parent filamentous fungal host cell that has not been modified, when measured under the same conditions. Preferably the modification is in the genome of the host cell.

Therefore, a mutant filamentous fungal host cell as defined herein is deficient in the cell in a polypeptide as described herein when
a) it has a modification which results in a reduced amount in the cell of a polypeptide as defined herewith or wherein said polypeptide is absent in the cell if compared to the parent filamentous fungal host cell that has not been modified, when measured under the same conditions and/or
b) It has a modification which results in a polypeptide with decreased or no activity if compared to the polypeptide in the parent filamentous fungal host cell that has not been modified, when measured under the same conditions.

In one embodiment the mutant filamentous fungal host cell according to the first aspect of the disclosure, wherein the mutant filamentous fungal host cell produces 1% less polypeptide as defined in claim 1 a. to 1 d. if compared with the parent filamentous fungal host cell which has not been modified and measured under the same conditions, at least 5% less, at least 10% less, at least 20% less, at least 30% less, at least 40% less, at least 50% less, at least 60% less, at least 70% less, at least 80% less, at least 90% less, at least 91% less, at least 92% less, at least 93% less, at least 94% less at least 95% less, at least 96% less, at least 97% less, at least 98% less, at least 99% less, or at least 99.9% less, preferably the mutant filamentous fungal host cell produces substantially no polypeptide as defined in the first aspect under 1 a. to 1 d. if compared with the parent filamentous fungal host cell which has not been modified when measured under the same conditions.

In one embodiment mutant filamentous fungal host cell according to the first aspect of the disclosure, wherein the mutant microbial host cell produces a polypeptide derived from the polypeptide as defined in the first aspect under 1 a. to 1 d. with 1% less activity, if compared with the parent filamentous fungal host cell which has not been modified and measured under the same conditions, at least 5% less activity, at least 10% less activity, at least 20% less activity, at least 30% less activity, at least 40% less activity, at least 50% less activity, at least 60% less activity, at least 70% less activity, at least 80% less activity, at least 90% less activity, at least 91% less activity, at least 92% less activity, at least 93% less activity, at least 94% less activity, at least 95% less activity, at least 96% less activity, at least 97% less activity, at least 98% less activity, at least 99% less activity, or at least 99.9% less activity, preferably the mutant filamentous fungal host cell produces a polypeptide derived from a polypeptide as defined in claim 1 a. to 1 d. with substantially no activity if compared with the parent filamentous fungal host cell which has not been modified when measured under the same conditions.

In one embodiment according to the first aspect of the disclosure, the modification, preferably a modification in the genome of the mutant filamentous fungal host cell, is selected from:
a. an insertion, deletion, or replacement of one or more nucleotides in a polynucleotide sequence coding for the polypeptide as defined herein or in a regulatory element required for transcription or translation of said polynucleotide sequence, to result in the reduced or no expression in the cell of the polypeptide as defined herein;
b. an insertion, deletion, or replacement of one or more nucleotides in the polynucleotide sequence coding for the polypeptide as defined herein or in a regulatory element required for transcription or translation of said polynucleotide sequence, to result in the expression in the cell of a polypeptide which is less active or has not activity if compared to the polypeptide as defined herein.

Deficiency of a mutant filamentous fungal host cell according to the disclosure in the production of a polypeptide as defined herein may be measured by determining the amount and/or (specific) activity of polypeptide as defined herein and/or it may be measured by determining the amount of mRNA transcribed from a polynucleotide encoding the polypeptide as described herein and/or it may be measured by gene or genome sequencing if compared to the parent filamentous fungal host cell which has not been modified. When deficiency of a mutant filamentous fungal host cell relates to deficiency in a polypeptide being a zinc binuclear cluster transcriptional regulator according to the disclosure, or DNA binding site thereof, said deficiency in the cell may also be measured by determining the concentration of oxalic acid produced extracellularly by the mutant filamentous fungal host cell, in comparison to the parent filamentous fungal host cell which has not been modified, e.g. using the assay as disclosed herein.

A modification in the genome can be determined by comparing the DNA sequence of the mutant filamentous fungal host cell to the sequence of the parent (non-modified) host cell. Sequencing of DNA and genome sequencing can be done using standard methods known to the person skilled in the art, for example using Sanger sequencing technology and/or next generation sequencing technologies such as Illumina GA2, Roche 454, etc. as reviewed in Elaine R. Mardis (2008), Next-Generation DNA Sequencing Methods, Annual Review of Genomics and Human Genetics, 9: 387-402. (doi: 10.1146/annurev.genom.9.081307.164359).

Deficiency in the production of the polypeptide as described herein can be measured using any assay suitable to the measurement of the polypeptide activity as defined herein available to the skilled person, transcriptional profiling, Northern blotting RT-PCR, Q-PCR and Western blotting. In particular, quantifying the amount of mRNA present in a cell may for example be achieved by northern blotting (in Molecular Cloning: A Laboratory Manual, Sambrook et al., New York: Cold Spring Harbour Press, 1989). Quantifying the amount of polypeptide as described herein present in a cell may for example be achieved by western blotting. The difference in mRNA amount may also be quantified by DNA array analysis (Eisen, M. B. and Brown, P. O. DNA arrays for analysis of gene expression. Methods Enzymol. 1999, 303:179-205).

A modification is construed as one or more modifications.

The modification in the filamentous fungal hosts cell can either be effected by
a) subjecting the parent filamentous fungal host cell to (classical) mutagenesis; and/or
b) subjecting the parent filamentous fungal host cell to recombinant genetic manipulation techniques or genome editing techniques; and/or
c) subjecting the parent filamentous fungal host cell to an inhibiting compound or composition.

Modification of a genome of a (mutant) filamentous fungal host cell is herein defined as any event resulting in a change in a polynucleotide sequence in the genome of the cell. In a preferred embodiment, the mutant microbial host cell according to the disclosure has a modification in its genome.

Modification can be introduced by classical strain improvement or random mutagenesis followed by selection or modification can also be introduced by site-directed mutagenesis with methods known to those skilled in the art.

Modification may be accomplished by the introduction (insertion), substitution (replacement) or removal (deletion) of one or more nucleotides in a polynucleotide sequence using recombinant genetic manipulation or genome editing techniques.

In one embodiment, the mutant filamentous fungal host cell according to the present disclosure comprises a modification in its genome selected from

a) a full or partial deletion of a polynucleotide as defined herein,
b) a full or partial replacement of a polynucleotide as defined herein with a polynucleotide sequence which does not code for a polypeptide as defined herein or which code for a partially or fully inactive form of a polypeptide as defined herein
c) a disruption of a polynucleotide as defined herein by the insertion of one or more nucleotides in the polynucleotide sequence.

This modification may for example be in a coding sequence or a regulatory element required for the transcription or translation of the polynucleotide as described above. For example, nucleotides may be inserted or removed to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32, (7) electronic access http://nar.oupjournals.org/cgi/reprint/32/7/e59 or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995, PO Box 1, Wymondham, Norfolk, U.K.).

Preferred methods of modification are based on recombinant genetic manipulation techniques and on genome editing techniques known to those skilled in the art. Introduction, deletion or replacement of one or more nucleotides in the genome of a filamentous fungus can be performed by homologous recombination, introducing a transforming DNA sequence on a (preferably linearized) vector comprising polynucleotide flanking sequences homologous to the locus to be modified. These methods are well known to those skilled in the art (Fincham Microbiol. Rev. (1989) 53(1): 148-170); Jeenes et al Biotechnology and Genetic Engineering Reviews (1991) 9: 327-367; Meyer, Biotechnology Advances (2008) 26: 177-185; Kick and Hoff, Appl. Microbiol. Biotechnol. (2010) 86: 51-62; Meyer et al “Genetics, Genetic Manipulation, and Approaches to Strain Improvement of Filamentous Fungi” in Manual of Industrial Microbiology and Biotechnology, 3^{rd} ed. (2010) 318-329). Alternatively, or in combination with other mentioned techniques, a technique based on in vivo recombination of cosmids in E. coli can be used, as described in: A rapid method for efficient gene replacement in the filamentous fungus Aspergillus nidulans (2000) Chaveroche, M-K., Ghico, J-M. and d'Enfert C; Nucleic acids Research, vol. 28, no 22. Alternatively, introduction, deletion or replacement of one or more nucleotides in the genome of a filamentous fungus can be performed using genome editing techniques such as e.g. CRISP-CAS9 mediated transformations (e.g. WO2016110453, WO2016100272, WO2016100568, Nodvig et al PLOS One (2015) DOI:10.1371/journal.pone.0133085).

Alternatively, modification, wherein said host cell produces less of or no protein such as the polypeptide as defined herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.).

In one embodiment, the mutant filamentous fungal host cell as disclosed herein is a mutant filamentous fungal host cell wherein the modification results in a reduced amount in the cell of (functional) mRNA encoding for a polypeptide as defined herein, if compared to the amount in the parent filamentous fungal cell which has not been modified, when measured under the same conditions.

A modification which results in a reduced amount of the mRNA encoding for the polypeptide as described herein may be obtained via methods that silence gene expression post-transcriptionally. These tools can be efficiently used when multiple copies of a gene to be deleted, disrupted, or replaced are present in a genome or when isogenes may compensate for knock-out of the deleted gene. A modification which results in a reduced amount of the mRNA coding for the polypeptide as described herein may be obtained via the RNA interference (RNAi) technique (FEMS Microb. Lett. 237 (2004): 317-324). In this method, identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in WO2008/053019, WO2005/05672A1, WO2005/026356A1, Oliveira et al, “Efficient cloning system for construction of gene silencing vectors in Aspergillus niger” (2008) Appl. Microbiol. and Biotechnol. 80 (5): 917-924 and/or Barnes et al, “siRNA as a molecular tool for use in Aspergillus niger” (2008) Biotechnology Letters 30 (5): 885-890 may be used at this purpose.

A modification which results in a polypeptide with decreased or no activity as defined herein can be obtained by different methods, for example by an antibody directed against such a polypeptide or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath et al, (2003) Nature: Systematic functional analysis of the Caenorhabditis elegans genome using RNAi. vol. 421, 231-237).

In addition of the above-mentioned techniques or as an alternative, it is also possible to inhibiting the activity of a polypeptide as defined herein, or to re-localize the polypeptide as defined herein by means of alternative signal sequences (Ramon de Lucas, J., Martinez O, Perez P., Isabel Lopez, M., Valenciano, S. and Laborda, F. The Aspergillus nidulans carnitine carrier encoded by the acuHgene is exclusively located in the mitochondria. FEMS Microbiol Lett. 2001 Jul. 24; 201(2):193-8.) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545.), or by targeting the polypeptide to a peroxisome which is capable of fusing with a membrane-structure of the cell involved in the secretory pathway of the cell, leading to secretion outside the cell of the polypeptide (e.g. as described in WO2006/040340).

Alternatively, or in combination with above-mentioned techniques, inhibition of polypeptide DNA binding activity, gene regulatory activity or enzymatic activity as defined herein can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol. Gen Genet. 1992 August; 234(2):332-6.) or using inhibitors inhibiting DNA binding activity, gene regulatory activity, enzymatic activity of a polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)).

In an embodiment according to the disclosure, the modification in the genome of the mutant microbial host cell according to the disclosure is a modification in at least one position of a polynucleotide as defined above encoding for the polypeptide, as defined above.

In the context of the present disclosure the “parent filamentous fungal host cell” and the “mutant filamentous fungal host cell” may be any type of filamentous fungal host cell. The specific embodiments of the mutant filamentous fungal host cell are hereafter described. It will be clear to those skilled in the art that embodiments applicable to the mutant filamentous fungal host cell are as well applicable to the parent filamentous fungal host cell unless otherwise indicated.

Filamentous fungi include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma.

Preferred filamentous fungal cells belong to a species of an Aspergillus, Acremonium, Myceliophthora, Thielavia Chrysosporium, Neurospora, Penicillium, Talaromyces, Rasamsonia, Fusarium, Humicola or Trichoderma genus, and most preferably a species of Aspergillus niger, Aspergillus awamori, Aspergillus flavus, Aspergillus bombycis, Aspergillus calidoustus, Aspergillus ochraceoroseus, Aspergillus nomius, Aspergillus aculeatus, Aspergillus carbonarius, Aspergillus parasiticus, Aspergillus kawachii, Aspergillus luchuensis, Aspergillus brasiliensis, Aspergillus foetidus, Aspergillus sojae, Aspergillus fumigatus, Aspergillus oryzae, Aspergillus nidulans, Aspergillus japonicus, Acremonium alabamense, Myceliophthora thermophila, Thielavia terrestris, Chrysosporium lucknowense, Chrysosporium inops, Chrysosporium keratinophilum, Chrysosporium merdarium, Chrysosporium pannicola, Chrysosporium queenslandicum, Chrysosporium tropicum, Chrysosporium zonatum, Fusarium oxysporum, Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Rasamsonia emersonii, Talaromyces emersonii, Trichoderma reesei, Trichoderma harzianum, Trichoderma koningii, Trichoderma longibrachiatum, Trichoderma viridae, Penicillium brasilianum, Penicillium camemberti, Penicillium rubens or Penicillium chrysogenum. A more preferred host cell belongs to the genus Aspergillus, more preferably the host cell belongs to the species Aspergillus niger. When the host cell according to the disclosure is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen und Zellkulturen GmbH (DSM), Westerdijk Fungal Biodiversity Institute formerly known as Centraalbureau Voor Schimmelcultures (CBS-KNAW), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia or the Fungal Genetics Stock Center (FGSC). Useful strains in the context of the present disclosure may be Aspergillus niger CBS 513.88 (available with FGSC under ref. number A1513), CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

According to one embodiment of the disclosure the mutant filamentous fungal host cell may further comprise one or more modifications, preferably in its genome, such that the mutant filamentous fungal host cell is deficient in the cell in at least one product selected from glucoamylase (glaA), acid stable alpha-amylase (amyA), neutral alpha-amylase (amyBI and amyBII), an α-1,3-glucan synthase (preferably AgsE or AgsE and AgsA), α-amylase AmyC (AmyC), a toxin, preferably ochratoxin and/or fumonisin, a protease transcriptional regulator prtT, PepA, a product encoded by the gene hdfA and/or hdfB, a non-ribosomal peptide synthase npsE if compared to a parent host cell and measured under the same conditions.

PrtT is a transcriptional regulator of proteases in eukaryotic cells. Several fungal transcriptional regulators of proteases have been recently described in WO 00/20596, WO 01/68864, WO 2006/040312 and WO 2007/062936. These transcriptional regulators were isolated from Aspergillus niger (A. niger), Aspergillus fumigatus (A. fumigatus), Penicillium chrysogenum (P. chrysogenum) and Aspergillus oryzae (A. oryzae). These transcriptional regulators of protease genes can be used to improve a method for producing a polypeptide in a fungal cell, wherein the polypeptide is sensitive for protease degradation. When the filamentous fungal host cell disclosed herein is deficient in prtT, the host cell will produce less proteases that are under transcriptional control of prtT. It is therefore advantageous when the host cell according to the disclosure is deficient in prtT. prtT deficient hosts and preferred methods to produce these hosts are extensively described in WO 01/68864, WO 2006/040312. WO 01/68864 and WO 2006/040312 describe recombinant and classic methods to disrupt the prtT coding sequence. WO 2007/062936 describes disruption of the prtT binding domain in a protease promoter. Disruption of the binding domain impedes binding of prtT to the binding domain. Consequently, the transcription of the protease is not activated by prtT and less protease is produced.

In one embodiment, the mutant filamentous fungal host cell disclosed herein comprises a modification, preferably in its genome, preferably in a polynucleotide coding for prtT, to result in a host cell which is deficient in prtT compared to a parent cell it originates from when cultivated under comparable conditions. Preferably, the prtT is a fungal prtT. More preferably, the prtT is the prtT from Aspergillus. Even more preferably the prtT is the prtT from Aspergillus niger. Even more preferably the prtT is the prtT from Aspergillus niger CBS 513.88. Most preferably, the prtT comprises the sequence of An04g06940.

The term “α-1,3-glucan synthase” refers herewith to an enzyme involved in the synthesis of 1,3-α-D-glucan, one of the main components in the cell wall of fungal species. Enzymes involved in the synthesis of 1,3-α-D-glucan have been isolated in many filamentous fungi. Examples of α-1,3-glucan synthases are e.g. AgsA (Genbank accession No. XM_658397) and agsB (Genbank accession No. XM_655819) from Aspergillus nidulans, ags1 (Genbank accession No. XM_743319) of Aspergillus fumigatus; agsB (Genbank accession No. AY530792) of Penicillium chrysogenum. AgsA, AgsB and AgsC from Aspergillus oryzae (Aspergillus database AspGD (http://www.aspergillusgenome.org) with gene numbers of agsA (AO090026000523), agsB (AO090003001500), and agsC (AO090010000106) genes), AgsA (An04g09890), AgsB, AgsC, AgsD and AgsE (An09g03070) from Aspergillus niger (Damveld et al, Fungal Genetics and Biology (2005) 42: 165-177; Yuan et al, Mol. Genet. Genomics (2008) 279: 545-561). It has been recently found that filamentous fungal host cells deficient in one or more α-1,3-glucan synthase have advantageous characteristics, e.g. the yield of a compound of interest produced which said cells is improved (WO2014013074 A1, WO2016066690 A1) and that the aggregation of cells is suppressed in the liquid culture of the filamentous fungus, and hence the filamentous fungus can be cultured at a high density., with an increased amount of a useful substance per unit volume (WO2014073674 A1).

In one embodiment, the mutant filamentous fungal host cell disclosed herein comprises a modification, preferably in its genome, preferably in a polynucleotide coding for an α-1,3-glucan synthase, to result in a cell which is deficient in α-1,3-glucan synthase compared to a parent cell it originates from when cultivated under comparable conditions. Preferably, the α-1,3-glucan synthase is from Aspergillus or Penicillium, such as AgsA, agsB from Aspergillus nidulans, ags of Aspergillus fumigatus; agsB of Penicillium chrysogenum. AgsA, AgsB and AgsC from Aspergillus oryzae, AgsA, AgsB, AgsC, AgsD and AgsE of A. niger. Even more preferably the α-1,3-glucan synthase is Aspergillus niger. Even more preferably it is AgsE and/or AgsA from Aspergillus niger CBS 513.88. Most preferably, it is AgsE comprising the sequence of An09g03070.

In one embodiment, the mutant filamentous fungal host cell disclosed herein comprises a modification, preferably in its genome, preferably in a polynucleotide encoding α-amylase amyC, resulting in a host cell which is deficient in the production of said α-amylase compared to a parent cell it originates from when cultivated under comparable conditions. Such α-amylase AmyC has described in WO2014013073 A1 with the polypeptide sequence according to SEQ ID NO: 3 or 7, the polynucleotide sequence according to SEQ ID NO: 1, 2, 5 or 6.

The term “glucoamylase” (glaA) is identical to the term “amyloglucosidase” and is defined herein as an enzyme having dextrin 6-alpha-D-glucanohydrolase activity which catalyzes the endo hydrolysis of 1, 6-alpha-D-glucoside linkages at points of branching in chains of 1, 4-linked alpha-D-glucose residues and terminal 1, 4-linked alpha-D-glucose residues. Glucoamylase activity can be measured as AGIU/ml by determining the liberation of paranitrofenol from the substrate p-nitrophenyl-α-D-glucopyranoside (Sigma). This results in a yellow color, whose absorbance can be measured at 405 nm using a spectrophotometer. 1 AGIU is the quantity of enzyme, which produces 1 μmole of glucose per minute at pH 4.3 and 60° C. from a soluble starch substrate. In WO98/46772 additional details of the assay can be found.

In one embodiment, the mutant filamentous fungal host cell disclosed herein comprises a modification, preferably in its genome, preferably in a polynucleotide encoding glaA, resulting in a host cell which is deficient in the production of glaA compared to a parent cell it originates from when cultivated under comparable conditions. Preferably, the glaA is a fungal glaA. More preferably, the glaA is the glaA from Aspergillus. Even more preferably the glaA is the glaA from Aspergillus niger. Even more preferably the glaA is the glaA from Aspergillus niger CBS 513.88. Most preferably, the glaA comprises the sequence of An03g06550.

The term “alpha-amylase” is defined herein as 1, 4-alpha-D-glucan glucanohydrolase activity which catalyzes the endohydrolysis of polysaccharides with three or more alpha-1, 4-linked glucose units in the presence of water to malto-oligosaccharides. To determine the (neutral) alpha-amylase activity, the Megazyme cereal alpha-amylase kit is used (Megazyme, CERALPHA alpha amylase assay kit, catalogus. ref. K-CERA, year 2000-2001), according a protocol of the supplier. The measured activity is based on hydrolysis of non-reducing-end blocked ρ-nitrophenyl maltoheptaoside in the presence of excess glucoamylase and α-glucosidase at a pH of 7.0. The amount of formed ρ-nitrophenol is a measure for alpha-amylase activity present in a sample.

The term “acid stable alpha-amylase” (amyA) is defined herein as an enzyme having alpha-amylase activity with optimal activity in the acid pH range. To determine the acid stable alpha-amylase activity, also the Megazyme cereal alpha-amylase kit is used (Megazyme, CERALPHA alpha amylase assay kit, catalogus. ref. K-CERA, year 2000-2001), according a protocol of the supplier but at an acid pH. The measured activity is based on hydrolysis of non-reducing-end-blocked ρ-nitrophenyl maltoheptaoside in the presence of excess glucoamylase and α-glucosidase at a pH of 4.5. The amount of formed ρ-nitrophenol is a measure for acid stable alpha-amylase activity present in a sample.

In one embodiment, the filamentous fungal host cell disclosed herein comprises a modification, preferably in its genome, preferably in a polynucleotide encoding AmyA, to result in a host cell which is deficient in amyA compared to the parent cell it originates from when cultivated under comparable conditions. Preferably, the amyA is a fungal amyA. More preferably, the amyA is the amyA from Aspergillus. Even more preferably the amyA is the amyA from Aspergillus niger. Even more preferably the amyA is the amyA from Aspergillus niger CBS 513.88. Most preferably, the amyA comprises the sequence of An11 g03340.

The term “neutral alpha-amylase activity” (amy) is defined herein as an enzyme having alpha-amylase activity with optimal activity in the neutral pH range.

In one embodiment, the filamentous fungal host cell disclosed herein comprises a modification, preferably in its genome, preferably in a polynucleotide encoding AmyB, to result in a host cell which is deficient in amyBI and/or amyBII compared to the parent cell it originates from when cultivated under comparable conditions. In one embodiment, the filamentous fungal host cell disclosed herein is deficient in amyBI and amy BII. Preferably, the amyB a is a fungal amyB. More preferably, the amyB is the amyB from Aspergillus. Even more preferably the amyB is the amyBI from Aspergillus niger. Even more preferably the amyB is the amyBI from Aspergillus niger CBS 513.88. Most preferably, the amyBI comprises the sequence of An12g06930. Even more preferably the amyB is the amyBII from Aspergillus niger. Even more preferably the amyB is the amyBII from Aspergillus niger CBS 513.88. Most preferably, the amyBII comprises the sequence of An05g02100.

The term toxin associated polynucleotide is defined herein as a gene cluster, a multitude of genes, a gene or part thereof encoding a compound, or biochemical pathway responsible for the biosynthesis or secretion of at least one toxin or toxin intermediate compound. Said compound may e.g. be a polypeptide, which may be an enzyme.

A number of host cells, especially fungi, which are used as host cells in the production of polypeptides of interest possesses genes encoding enzymes involved in the biosynthesis of various toxins. For example, cyclopiazonic acid, kojic acid, 3-nitropropionic acid and aflatoxins are known toxins, which are formed in, e.g., Aspergillus flavus. Similarly, trichothecenes are formed in a number of fungi, e.g., in Fusarium sp. such as Fusarium venenatum and in Trichoderma and ochratoxin may be produced by Aspergillus. Recently, sequencing of the genome of an industrial Aspergillus niger host strain revealed a fumonisin gene cluster (Pel et al., “Genome sequencing and analysis of the versatile cell factory Aspergillus niger CBS 513.88”. Nat Biotechnol. 2007 February; 25 (2):221-231). The formation of such toxins during the fermentation of compounds of interest is highly undesirable as these toxins may present a health hazard to operators, customers and the environment. Consequently, a toxin deficient host cell enables toxin-free production of a compound of interest. The toxin-free compound is easier to produce since no toxin has to be removed from the product. Furthermore, the regulatory approval procedure for the compound is easier.

In one embodiment, the filamentous fungal host cell disclosed herein comprises a modification, preferably in its genome, preferably in a toxin associated polynucleotide encoding a compound (which may e.g. be a polypeptide which may be an enzyme) or biochemical pathway, to result in a host cell which is deficient in the production of said toxin or a toxin intermediate compound compared to the parent cell it originates from when cultivated under comparable conditions. Preferably, the toxin or toxin intermediate compound is a fungal toxin or toxin intermediate compound. More preferably, the toxin or toxin intermediate compound is a toxin or toxin intermediate compound from Aspergillus. Even more preferably the toxin or the toxin intermediate compound is a toxin or toxin intermediate compound from Aspergillus niger. Even more preferably the toxin or toxin intermediate compound is a toxin or toxin intermediate compound from Aspergillus niger CBS 513.88. Even more preferably, the toxin or the toxin intermediate compound is fumonisin or a fumonisin intermediate compound. Even more preferably, the toxin or the toxin intermediate compound is ochratoxin or an ochratoxin intermediate compound. Most preferably, the toxin or the toxin intermediate compound is ochratoxin or fumonisin or an ochratoxin or a fumonisin intermediate compound.

Preferably, the toxin associated polynucleotide encodes a compound (which may e.g. be a polypeptide which may be an enzyme) or a biochemical pathway which is involved in the production of a fungal toxin or toxin intermediate compound. More preferably, a toxin or toxin intermediate compound from Aspergillus. Even more preferably, a toxin or toxin intermediate compound from Aspergillus niger. Even more preferably, a toxin or toxin intermediate compound from Aspergillus niger CBS 513.88. Even more preferably, a fumonisin or a fumonisin intermediate compound. Even more preferably, a fumonisin-B or a fumonisin-B intermediate compound. Even more preferably, a fumonisin-B2 or a fumonisin-B2 intermediate compound. Even more preferably, the toxin associated polynucleotide comprises the sequence of the fumonisin cluster from An01 g06820 until An01g06930. Most preferably, the toxin associated polynucleotide comprises the sequence of An01 g06930.

In another preferred embodiment, the toxin associated polynucleotide encodes a compound (which may e.g. be a polypeptide which may be an enzyme) or a biochemical pathway which is involved in ochratoxin or an ochratoxin intermediate compound. More preferably, an ochratoxin A or an ochratoxin A intermediate compound. More preferably, the toxin associated polynucleotide comprises the sequence of the cluster from An15g07880 until An15g07930. Most preferably, the toxin associated polynucleotide comprises the sequence of An15g07910 and/or the sequence of An15g07920.

In one embodiment, the filamentous fungal host cell disclosed herein comprises at least one modification, preferably in its genome, preferably in a toxin associated polynucleotide encoding a compound (which may e.g. be a polypeptide which may be an enzyme) or biochemical pathway, resulting in a host cell which is deficient in the production of a toxin or, toxin intermediate compound compared to the parent cell it originates from when cultivated under comparable conditions.

In one embodiment, the filamentous fungal host cell disclosed herein comprises at least two modifications, preferably in its genome, preferably in two toxin associated polynucleotides, to result in a host cell which is preferably deficient in the production of fumonisin and ochratoxin compared to the parent cell it originates from when cultivated under comparable conditions.

In one embodiment, the filamentous fungal host cell disclosed herein comprises three or more modifications, preferably in its genome, preferably in three or more toxin associated polynucleotides, to result in a host cell which is preferably deficient in the production of fumonisin, ochratoxin and at least one additional toxin or toxin intermediate compound compared to the parent cell it originates from when cultivated under comparable conditions.

In one embodiment, the filamentous fungal host cell disclosed herein may comprise one or more modifications, preferably in its genome, to result in a deficiency in the production of the major extracellular aspartic protease PepA. For example, the host cell as disclosed herein may further comprise a disruption of the pepA gene encoding the major extracellular aspartic protease PepA. More preferably, the pepA is the pepA from Aspergillus. Even more preferably the pepA is the pepA from Aspergillus niger. Even more preferably the pepA is the pepA from Aspergillus niger CBS 513.88. Most preferably, the pepA comprises the sequence of An14g04710.

Preferably, the efficiency of targeted integration of a polynucleotide to a pre-determined site into the genome of the mutant filamentous fungal host cell as disclosed herein is increased by making the cell deficient in a component in NHR (non-homologous recombination). Preferably, the mutant filamentous fungal host cell as disclosed herein comprises a modification, preferably in its genome, preferably in a polynucleotide encoding an NHR component, to result in a host cell which is deficient in the production of said NHR component compared to a parent cell it originates from when cultivated under the same conditions. In this context, the NHR may be any NHR component known to the person skilled in the art. Preferred NHR components are selected from the group of filamentous fungal homologues of yeast KU70, KU80, MRE11, RAD50, RAD51, RAD52, XRS2, SIR4, LIG4. More preferred NHR components are filamentous fungal homologues of yeast KU70 and KU80, preferably hdfA (homologue of yeast KU70) or homologues thereof and hdfB (homologue of yeast KU80) or homologues thereof. The most preferred NHR component in this context is KU70 or hdfA, or a homologue thereof. Another preferred NHR component in this context is KU80 or hdfB, or a homologue thereof. Methods to obtain such host cell deficient in a component involved in NHR are known to the skilled person and are extensively described in WO2005095624. Preferably the hdfA gene is the hdfA gene from A. niger, more preferably the hdfA from A. niger according to SEQ ID NO: 1 of WO2005/095624. In another preferred embodiment, the hdfB gene is the hdfB gene from A. niger, more preferably the hdfB from A. niger according to SEQ ID NO: 4 of WO2005095624.

Therefore, the mutant filamentous fungal host cell as disclosed herein may additionally comprises one or more modifications, preferably in its genome, to result in a deficiency in the production of the product encoded by the hdfA gene (as depicted in SEQ ID NO: 3 of WO 2005/095624) and/or hdfB gene (as depicted in SEQ ID NO: 6 of WO 2005095624). For example, the host cell according to the disclosure may further comprise a disruption or deletion of the hdfA and/or hdfB gene. Filamentous fungal host cells which are deficient in a product encoded by the hdfA and/or hdfB gene have been described in WO 2005095624.

The mutant filamentous fungal host cell as disclosed herein may additionally comprise a modification, preferably in its genome, which results in the deficiency in the production of the non-ribosomal peptide synthase npsE. Such host cells deficient in the production of non-ribosomal peptide synthase npsE have been described in WO2012001169 (npsE has a genomic sequence as depicted in SEQ ID NO: 35, a coding sequence depicted in SEQ ID NO: 36, the mRNA depicted in SEQ ID NO: 37 and the nrps protein depicted in SEQ ID NO: 38 of WO2012001169).

The deficiency in the production of at least one product selected from glucoamylase (glaA), acid stable alpha-amylase (amyA), neutral alpha-amylase (amyBI and amyBII), α-1,3-glucan synthase (AgsE and/or AgsA), a toxin, preferably ochratoxin and/or fumonisin, a protease transcriptional regulator prtT, PepA, a product encoded by the gene hdfA and/or hdfB, a non-ribosomal peptide synthase npsE, amylase amyC if compared to a parent host cell and measured under the same conditions may already be present in the parent host cell from which the mutant microbial host cell according to the disclosure is derived.

In one embodiment the mutant filamentous fungal host cell as disclosed herein further comprises a deficiency in the production of glaA and optionally at least another product selected from the group consisting of acid stable alpha-amylase (amyA), neutral alpha-amylase (amyBI and amyBII), α-1,3-glucan synthase (AgsE and/or AgsA), a toxin, preferably ochratoxin and/or fumonisin, a protease transcriptional regulator prtT, PepA, a product encoded by the gene hdfA and/or hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA and optionally at least another product selected from the group consisting of acid stable alpha-amylase (amyA), neutral alpha-amylase (amyBI and amyBII), α-1,3-glucan synthase (AgsE and/or AgsA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA) and optionally at least another product selected from the group consisting of neutral alpha-amylase (amyBI and amyBII), α-1,3-glucan synthase (AgsE and/or AgsA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and optionally at least another product selected from the group consisting of neutral alpha-amylase amyBII, α-1,3-glucan synthase (AgsE and/or AgsA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, and optionally at least another product selected from the group consisting of α-1,3-glucan synthase (AgsE and/or AgsA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, a product encoded by the gene hdfA and optionally at least another product selected from the group consisting of α-1,3-glucan synthase (AgsE and/or AgsA), a toxin, preferably ochratoxin and/or fumonisin, a protease transcriptional regulator prtT, a product encoded by the gene hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, a product encoded by the gene hdfA, α-1,3-glucan synthase (AgsE and/or AgsA), and optionally at least another product selected from the group consisting of, a toxin, preferably ochratoxin and/or fumonisin, a protease transcriptional regulator prtT, a product encoded by the gene hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, a product encoded by the gene hdfA, α-1,3-glucan synthase (AgsE and/or AgsA), ochratoxin, fumonisin, and optionally at least another product selected from the group consisting of a protease transcriptional regulator prtT, a product encoded by the gene hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, a product encoded by the gene hdfA, α-1,3-glucan synthase (AgsE and/or AgsA), ochratoxin, fumonisin, a protease transcriptional regulator prtT and optionally at least another product selected from the group consisting of a product encoded by the gene hdfB, a non-ribosomal peptide synthase npsE, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, a product encoded by the gene hdfA, α-1,3-glucan synthase (AgsE and/or AgsA), ochratoxin, fumonisin, a protease transcriptional regulator prtT, a non-ribosomal peptide synthase npsE and optionally at least another product selected from the group consisting of a product encoded by the gene hdfB, amylase amyC if compared to a parent host cell and measured under the same conditions.

In one embodiment the mutant filamentous fungal host cell as disclosed herein further comprises a deficiency in the production of glaA, PepA, acid stable alpha-amylase (amyA), neutral alpha-amylase amyBI and amyBII, a product encoded by the gene hdfA, α-1,3-glucan synthase (AgsE and/or AgsA), ochratoxin, fumonisin, a protease transcriptional regulator prtT, amylase amyC and optionally at least another product selected from the group consisting of a product encoded by the gene hdfB, a non-ribosomal peptide synthase npsE, if compared to a parent host cell and measured under the same conditions.

In one embodiment, the mutant filamentous fungal host cell as disclosed herein further has a reduced amylase background and comprises a deficiency in the production of glaA, acid stable alpha-amylase (amyA), neutral alpha-amylase amyBI and amyBII, if compared to a parent host cell and measured under the same conditions. Such a mutant host cell may also comprise a deficiency in a filamentous fungal homolog of KU70 or KU80. Such a microbial mutant cell may also comprise a deficiency in the production of a toxin. Such a microbial mutant cell may also comprise a deficiency in the production of a filamentous fungal homolog of KU70 or KU80 and a deficiency in the production of a toxin.

In one embodiment, the mutant filamentous fungal host cell as disclosed herein further has a reduced amylase background and comprises a deficiency in the production of glaA, acid stable alpha-amylase (amyA), neutral alpha-amylase amyBI and amyBII, if compared to a parent host cell and measured under the same conditions. Such mutant host cell may also comprise a deficiency in a filamentous fungal homolog of KU70 or KU80. Such a mutant host cell may also comprise a deficiency in a toxin. Such a mutant host cell may also comprise a deficiency in a filamentous fungal homolog of KU70 or KU80 and a deficiency in the production of a toxin. Such a mutant host cell may also comprise a deficiency in α-1,3-glucan synthase AgsE and/or AgsA.

In one embodiment, the mutant filamentous fungal host cell as disclosed herein has a reduced amylase background and further comprises a deficiency in glaA, acid stable alpha-amylase (amyA), neutral alpha-amylase amyBI, amyBII and amyC if compared to a parent host cell and measured under the same conditions. Such a mutant host cell may also comprise a deficiency in a filamentous fungal homolog of KU70 or KU80. Such a mutant host cell may also comprise a deficiency in the production of a toxin. Such a mutant host cell may also comprise a deficiency in the production of a filamentous fungal homolog of KU70 or KU80 and a deficiency in the production of a toxin. Such a mutant host cell may also comprise a deficiency in α-1,3-glucan synthase AgsE and/or AgsA.

In one embodiment, the mutant filamentous fungal host cell as disclosed herein further has a reduced alpha-amylase background and comprises a deficiency in the production acid stable alpha-amylase (amyA), neutral alpha-amylase amyBI and amyBII and, optionally, amyC if compared to a parent host cell and measured under the same conditions. Such a mutant host cell may also comprise a deficiency in a filamentous fungal homolog of KU70 or KU80. Such a mutant host cell may also comprise a deficiency in a toxin. Such a mutant host cell may also comprise a deficiency in a filamentous fungal homolog of KU70 or KU80 and a deficiency in a toxin. Such a mutant host cell may also comprise a deficiency in aα-1,3-glucan synthase AgsE and/or AgsA.

The mutant filamentous fungal host cell as disclosed herein may additionally comprise at least two substantially homologous DNA domains suitable for integration of one or more copies of a polynucleotide encoding a compound of interest wherein at least one of the at least two substantially homologous DNA domains is adapted to have enhanced integration preference for the polynucleotide encoding a compound of interest compared to the substantially homologous DNA domain it originates from, and wherein the substantially homologous DNA domain where the adapted substantially homologous DNA domain originates from has a gene conversion frequency that is at least 10% higher than one of the other of the at least two substantially homologous DNA domains. These cells have been described in WO2011/009700. Strains containing two or more copies of these substantially homologous DNA domains are also referred hereafter as strain containing two or more amplicons. Examples of host cells comprising such amplicons are e.g. described in van Dijck et al, 2003, Regulatory Toxicology and Pharmacology 28; 27-35: On the safety of a new generation of DSM Aspergillus niger enzyme production strains. In van Dijck et al, an Aspergillus niger strain is described that comprises 7 amplified glucoamylase gene loci, i.e. 7 amplicons. Preferred host cells within this context are filamentous fungus host cells, preferably A. niger host cells, comprising two or more amplicons, preferably two or more ΔglaA amplicons (preferably comprising 3, 4, 5, 6, 7 ΔglaA amplicons) wherein the amplicon which has the highest frequency of gene conversion, has been adapted to have enhanced integration preference for the polynucleotide encoding a compound of interest compared to the amplicon it originates from. Adaptation of the amplicon can be performed according to any one of the methods described in WO2011009700 (which is here fully incorporated by reference). An example of these host cells, described in WO2011009700, are host cells comprising three ΔglaA amplicons being a BamHI truncated amplicon, a Sail truncated amplicon and a BgIII truncated amplicon and wherein the BamHI amplicon has been adapted to have enhanced integration preference for a polynucleotide encoding a compound of interest compared to the BamHI amplicon it originates from. Host cells comprising two or more amplicons wherein one amplicon has been adapted to have enhanced integration preference for a polynucleotide encoding a compound of interest compared to the amplicon it originates from are hereafter referred as host cells comprising an adapted amplicon.

The mutant filamentous fungal host cell as disclosed herein may additionally comprises a modification of Sec61. A preferred SEC61 modification is a modification which results in a one-way mutant of SEC61; i.e. a mutant wherein the de novo synthesized protein can enter the ER via SEC61, but the protein cannot leave the ER via SEC61. Such modifications are extensively described in WO2005/123763. In one embodiment, the mutant host cell comprises a modification in a Sec61 as depicted in SEQ ID NO: 3 of WO2005123763. Most preferably, the SEC 61 modification is the S376W mutation in which Serine 376 is replaced by Tryptophan in SEQ ID NO: 3 of WO2005123763.

In one embodiment, according to the first aspect of the disclosure the mutant filamentous fungal host cell comprises at least one polynucleotide coding for a compound of interest or at least one polynucleotide coding for a compound involved in the production of a compound of interest

In another embodiment, the at least one polynucleotide coding for the compound of interest or the at least one polynucleotide coding for a compound involved in the production of a compound of interest is operably linked to a promoter capable to promote the expression of said polypeptide in the host cell.

The compound of interest can be any biological compound. The biological compound may be biomass or a biopolymer or metabolite, preferably the compound of interest is selected from a biopolymer or a metabolite. The biological compound may be encoded by a single polynucleotide or a series of polynucleotides composing a biosynthetic or metabolic pathway or may be the direct result of the product of a single polynucleotide or products of a series of polynucleotides. The biological compound may be native to the host cell or heterologous.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide, polyamide, or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. Polypeptides further include naturally occurring allelic and engineered variations of the above-mentioned polypeptides and hybrid polypeptides. The polypeptide may be native or may be heterologous to the host cell. The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, or protein. The protein may be an enzyme such as, a protease, ceramidases, epoxide hydrolase, aminopeptidase, acylases, aldolase, hydroxylase, aminopeptidase, lipase. The polypeptide may also be an enzyme secreted extracellularly. Enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase, monooxygenase.

According to the disclosure, a polypeptide or enzyme may also be a product as described in WO2010102982. A polypeptide may also be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter (s) and terminator. The hybrid polypeptides may comprise a combination of partial or complete polypeptide sequences obtained from at least two different polypeptides wherein one or more may be heterologous to the host cell. Example of fusion polypeptides and signal sequence fusions are for example as described in WO2010/121933.

The biopolymer may be a polysaccharide. The polysaccharide may be any polysaccharide, including, but not limited to, a mucopolysaccharide (e. g., heparin and hyaluronic acid) and nitrogen-containing polysaccharide (e.g., chitin). Polysaccharide may be hyaluronic acid.

The polynucleotide coding for the compound of interest or coding for a compound involved in the production of the compound of interest as disclosed herein may encode an enzyme involved in the synthesis of a primary or secondary metabolite, such as organic acids, carotenoids, (beta-lactam) antibiotics, and vitamins.

The term “metabolite” encompasses both primary and secondary metabolites; the metabolite may be any metabolite. Preferred metabolites are citric acid, gluconic acid, adipic acid, fumaric acid, itaconic acid and succinic acid.

The metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

The primary metabolite may be, but is not limited to, an amino acid, fatty acid, nucleoside, nucleotide, sugar, triglyceride, or vitamin.

The secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The biological compound may also be a selectable marker or the product of a selectable marker but preferably the biological compound is not a selectable marker and/or it is not the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof.

The mutant filamentous fungal host cell may already be capable of producing the compound of interest. The mutant filamentous fungal host cell may also be provided with a homologous or heterologous nucleic acid construct that encodes a polypeptide wherein the polypeptide may be the compound of interest or a polypeptide involved in the production of the compound of interest. The person skilled in the art knows how to modify a host cell such that it is becomes capable of producing the compound of interest.

The host cell may be transformed with a nucleic acid construct comprising the polynucleotide coding for the compound of interest or coding for a compound involved in the synthesis of a compound of interest.

The term “nucleic acid construct” is herein referred to as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” (or expression construct) when the nucleic acid construct contains all the control sequences required for expression of a coding sequence, wherein said control sequences are operably linked to said coding sequence.

The term “operably linked” is defined herein as a configuration in which a control sequence is appropriately placed at a position relative to the coding sequence of the DNA sequence such that the control sequence directs the production of an RNA or an mRNA and optionally of a polypeptide translated from said (m)RNA.

The term “control sequences” is defined herein to include all components, which are necessary or advantageous for the expression of mRNA and/or a polypeptide, either in vitro or in a host cell. Each control sequence may be native or foreign to the nucleic acid sequence encoding the polypeptide. Such control sequences include, but are not limited to, a leader, Shine-Delgarno sequence, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription terminator. At a minimum, the control sequences include a promoter, and transcriptional and translational stop signals. Control sequences may be optimized to their specific purpose. Preferred optimized control sequences used in the present disclosure are those described in WO2006077258, which is herein incorporated by reference.

The control sequences may be provided with linkers for introducing specific restriction sites facilitating ligation of the control sequences with the coding region of the nucleic acid sequence encoding a polypeptide.

The control sequence may be an appropriate promoter sequence (promoter).

The control sequence may also be a suitable transcription terminator (terminator) sequence, a sequence recognized by a filamentous fungal cell to terminate transcription. The terminator sequence is operably linked to the 3′-terminus of the nucleic acid sequence encoding the polypeptide. Any terminator, which is functional in the cell, may be used in the present disclosure. The man skilled in the art knows which types of terminators can be used in the microbial host cell as described herein.

Preferred terminator sequences for filamentous fungal cells are obtained from any terminator sequence of a filamentous fungal gene, more preferably from Aspergillus genes, even more preferably from the gene A. oryzae TAKA amylase, the genes encoding A. nigerglucoamylase (glaA), A. nidulans anthranilate synthase, A. niger alpha-glucosidase, trpC and/or Fusarium oxysporum trypsin-like protease.

The control sequence may also be an optimal translation initiation sequences (as described in Kozak, 1991, J. Biol. Chem. 266:19867-19870), or a 5′-untranslated sequence, a non-translated region of a mRNA which is important for translation by the mutated microbial host cell. The translation initiation sequence or 5′-untranslated sequence is operably linked to the 5′-terminus of the coding sequence encoding the polypeptide. Each control sequence may be native or foreign to the nucleic acid sequence encoding the polypeptide. Control sequences may be optimized to their specific purpose.

Suitable 5′-untranslated sequences may be those polynucleotides preceding the fungal amyloglucosidase (AG) gene, A. oryzae TAKA amylase and Aspergillus triose phosphate isomerase genes and A. nigerglucoamylase glaA, alpha-amylase, xylanase and phytase encoding genes.

The control sequence may also be a non-translated region of a mRNA which is important for translation by the mutated microbial host cell. The leader sequence is operably linked to the 5′-terminus of the nucleic acid sequence encoding the polypeptide. Any leader sequence, which is functional in the cell, may be used in the present disclosure.

Leader sequences may be those originating from the fungal amyloglucosidase (AG) gene (glaA-both 18 and 24 amino acid versions e. g. from Aspergillus). Preferred leaders for filamentous fungal cells are obtained from the polynucleotides preceding A. oryzae TAKA amylase and A. nidulans triose phosphate isomerase and A. niger glaA and phytase.

Other control sequences may be isolated from the Penicillium IPNS gene, or pcbC gene, the beta tubulin gene. All the control sequences cited in WO 01/21779 are herewith incorporated by reference.

The control sequence may also be a polyadenylation sequence, a sequence which is operably linked to the 3′-terminus of the nucleic acid sequence and which, when transcribed, is recognized by the microbial host cell (mutated or parent) as a signal to add poly-adenosine residues to transcribed mRNA. Any polyadenylation sequence, which is functional in the cell, may be used in the present disclosure.

Preferred polyadenylation sequences for filamentous fungal cells are obtained from the polynucleotides encoding A. oryzae TAKA amylase, A. niger glucoamylase, A. nidulans anthranilate synthase, Fusarium oxysporum trypsin-like protease and A. niger alpha-glucosidase.

In an embodiment, in the mutant filamentous fungal host cell as disclosed herein the at least one polynucleotide coding for the compound of interest or the at least one polynucleotide coding for a compound involved in the production of a compound of interest is operably linked to a promoter, capable to promote the expression of said polypeptide in the host cell.

The term “promoter” is defined herein as a DNA sequence that binds RNA polymerase and directs the polymerase to the correct downstream transcriptional start site of a nucleic acid sequence encoding a biological compound to initiate transcription. RNA polymerase effectively catalyzes the assembly of messenger RNA complementary to the appropriate DNA strand of a coding region. The term “promoter” will also be understood to include the 5′-non-coding region (between promoter and translation start) for translation after transcription into mRNA, cis-acting transcription control elements such as enhancers, and other nucleotide sequences capable of interacting with transcription factors. The promoter may be any appropriate promoter sequence suitable for a eukaryotic or prokaryotic host cell, which shows transcriptional activity, including mutant, truncated, and hybrid promoters, and may be obtained from polynucleotides encoding extra-cellular or intracellular polypeptides either homologous (native) or heterologous (foreign) to the cell. The promoter may be a constitutive or inducible promoter.

Promoters suitable in filamentous fungi are promoters which may be selected from the group, which includes but is not limited to promoters obtained from the polynucleotides encoding A. oryzae TAKA amylase, Rhizomucor miehei aspartic proteinase, Aspergillus gpdA promoter, A. niger neutral alpha-amylase, A. niger acid stable alpha-amylase, A. niger or A. awamori glucoamylase (glaA), A. niger or A. awamori endo-xylanase (xlnA) or beta-xylosidase (xlnD), T. reesei cellobiohydrolase I (CBHI), R. miehei lipase, A. oryzae alkaline protease, A. oryzae triose phosphate isomerase, A. nidulans acetamidase, Fusarium venenatum amyloglucosidase (WO 00/56900), Fusarium venenatum Dania (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase), and mutant, truncated, and hybrid promoters thereof. Other examples of promoters are the promoters described in WO2006092396 and WO2005100573, or in WO2008/098933. Inducible promoters which can be used in filamentous fungi are the A. oryzae TAKA amylase, A. niger neutral alpha-amylase, A. niger acid stable alpha-amylase, A. niger or A. awamori glucoamylase (glaA), A. niger or A. awamori endo-xylanase (xlnA) or beta-xylosidase (xlnD), T., Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase) as defined above.

Preferably the promoter sequence is from a highly-expressed gene. Specific examples of suitable highly expressed genes include e. g. the methanol oxidase genes (AOX and MOX) from Hansenula and Pichia, respectively, the glucoamylase (glaA) genes from A. niger and A. awamori, the A. oryzae TAKA-amylase gene, the A. nidulans gpdA gene and the T. reesei cellobiohydrolase genes.

All above-mentioned promoters are readily available in the art.

To facilitate expression, the polynucleotide encoding the polypeptide being the compound of interest or the polypeptide involved in the production of the compound of interest may be a synthetic polynucleotide. The synthetic polynucleotides may be optimized in codon use, preferably according to the methods described in WO2006077258 and/or WO2008/000632 (the latter addressing codon-pair optimization). Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence.

The nucleic acid construct has defined herewith may be an expression vector.

The expression vector may be any vector (e.g., a plasmid or virus), which can be conveniently subjected to recombinant DNA procedures and can bring about the expression of the polynucleotide encoding the polypeptide. The choice of the vector will typically depend on the compatibility of the vector with the cell into which the vector is to be introduced. The vectors may be linear or closed circular plasmids. The vector may be an autonomously replicating vector, i.e., a vector, which exists as an extra-chromosomal entity, the replication of which is independent of chromosomal replication, e.g., a plasmid, an extra-chromosomal element, a mini-chromosome, or an artificial chromosome. An autonomously maintained cloning vector may comprise the AMA1-sequence (see e.g. Aleksenko and Clutterbuck (1997), Fungal Genet. Biol. 21: 373-397).

Alternatively, the vector may be one which, when introduced into the host cell, is integrated into the genome, and replicated together with the chromosome(s) into which it has been integrated. The integrative cloning vector may integrate at random or at a predetermined target locus in the chromosomes of the host cell. In a preferred embodiment of the disclosure, the integrative cloning vector comprises a DNA fragment, which is homologous to a DNA sequence in a predetermined target locus in the genome of host cell for targeting the integration of the cloning vector to this predetermined locus. To promote targeted integration, the cloning vector is preferably linearized prior to transformation of the cell. Linearization is preferably performed such that at least one but preferably both ends of the cloning vector are flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb. Preferably, the efficiency of targeted integration into the genome of the host cell, i.e. integration in a predetermined target locus, is increased by improving homologous recombination abilities of the host cell.

Preferably, the homologous flanking DNA sequences in the cloning vector, which are homologous to the target locus, are derived from a highly-expressed locus meaning that they are derived from a gene, which is capable of high expression level in the host cell. A gene capable of high expression level, i.e. a highly-expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1).

A few preferred highly expressed fungal genes are given as example: the amylase, glucoamylase, alcohol dehydrogenase, xylanase, glyceraldehyde-phosphate dehydrogenase or cellobiohydrolase (cbh) genes from Aspergilli, Chrysosporium or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1, a Chrysosporium lucknowense cbh gene or a cbh gene from P. chrysogenum.

More than one copy of a nucleic acid sequence may be inserted into the mutated microbial host cell to increase production of the product (over-expression) encoded by said sequence. This can be done, preferably by integrating into its genome copies of the DNA sequence, more preferably by targeting the integration of the DNA sequence at one of the highly-expressed loci defined in the former paragraph. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase even more the number of copies of the DNA sequence to be over expressed the technique of gene conversion as described in WO98/46772 may be used.

The vector system may be a single vector or plasmid or two or more vectors or plasmids, which together contain the total DNA to be introduced into the genome of the host cell, or a transposon.

The vectors preferably contain one or more selectable markers, which permit easy selection of transformed cells. A selectable marker is a gene the product of which provides for biocide or viral resistance, resistance to heavy metals, prototrophy to auxotrophs, and the like. The selectable marker may be introduced into the cell on the expression vector as the expression cassette or may be introduced on a separate expression vector.

A selectable marker for use in a filamentous fungal cell may be selected from the group including, but not limited to, amdS (acetamidase), argB (ornithine carbamoyltransferase), bar (phosphinothricinacetyltransferase), bleA (phleomycin binding), hygB (hygromycinphosphotransferase), niaD (nitrate reductase), pyrG (orotidine-5′-phosphate decarboxylase), sC (sulfate adenyltransferase), NAT or NTC (Nourseothricin) and trpC (anthranilate synthase), as well as equivalents from other species. Preferred for use in an Aspergillus and Penicillium cell are the amdS (see for example EP 635574 B1, EP0758020A2, EP1799821A2, WO 97/06261A2) and pyrG genes of A. nidulans or A. oryzae and the bar gene of Streptomyces hygroscopicus. More preferably an amdS gene is used, even more preferably an amdS gene from A. nidulans or A. niger. A most preferred selectable marker gene is the A. nidulans amdS coding sequence fused to the A. nidulans gpdA promoter (see EP 635574 B1). Other preferred AmdS markers are those described in WO2006/040358. AmdS genes from other filamentous fungi may also be used (WO 97/06261).

In a preferred embodiment, the selection marker is deleted from the transformed host cell after introduction of the expression construct so as to obtain transformed host cells capable of producing the polypeptide which are free of selection marker genes.

The procedures used to ligate the elements described above to construct the recombinant expression vectors as disclosed herein are well known to one skilled in the art (see, e.g. Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995).

Furthermore, standard molecular cloning techniques such as DNA isolation, gel electrophoresis, enzymatic restriction modifications of nucleic acids, Southern analyses, transformation of cells, etc., are known to the skilled person and are for example described by Sambrook et al. (1989) “Molecular Cloning: a laboratory manual”, Cold Spring Harbor Laboratories, Cold Spring Harbor, N.Y., and Innis et al. (1990) “PCR protocols, a guide to methods and applications” Academic Press, San Diego.

A nucleic acid may be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis.

Optionally, the host cell has been modified to comprise an elevated unfolded protein response (UPR) to enhance production abilities of a polypeptide of interest. UPR may be increased by techniques described in US2004/0186070A1 and/or US2001/0034045A1 and/or WO01/72783A2 and/or WO2005/123763. More specifically, the protein level of HAC1 and/or IRE1 and/or PTC2 may be modulated, and/or the SEC61 protein may be engineered to obtain a host cell having an elevated UPR.

The person skilled in the art knows how to transform cells with the one or more expression cassettes and the selectable marker. For example, the skilled person may use one or more expression vectors, wherein the one or more cloning vectors comprise the expression cassettes and the selectable marker.

Any suitable known methods can be used for the transformation of the mutant filamentous fungal host cell as disclosed herein, including e.g. electroporation methods, particle bombardment or microprojectile bombardment, protoplast methods and Agrobacterium mediated transformation (AMT). Preferably the protoplast method is used. Procedures for transformation are described by J. R. S. Fincham, Transformation in fungi. 1989, Microbiological reviews. 53, 148-170.

Transformation may involve a process consisting of protoplast formation, transformation of the protoplasts, and regeneration of the cell wall in a manner known per se. Suitable procedures for transformation of Aspergillus cells are described in EP 238 023 and Yelton et al., 1984, Proceedings of the National Academy of Sciences USA 81:1470-1474. Suitable procedures for transformation of Aspergillus and other filamentous fungal host cells using Agrobacterium tumefaciens are described in e.g. De Groot et al., Agrobacterium tumefaciens-mediated transformation of filamentous fungi. Nat Biotechnol. 1998, 16:839-842. Erratum in: Nat Biotechnol 1998 16:1074. A suitable method of transforming Fusarium species is described by Malardier et al., 1989, Gene 78:147156 or in WO 96/00787. Other methods can be applied such as a method using biolistic transformation as described in: Christiansen et al., Biolistic transformation of the obligate plant pathogenic fungus, Erysiphe graminis f. sp. hordei. 1995, Curr Genet. 29:100-102.

In order to enhance the amount of copies of the polynucleotide coding for the compound of interest or coding for a compound involved in the production by the cell of the compound of interest (the gene, multiple transformations of the host cell may be required. In this way, the ratios of the different enzymes produced by the host cell may be influenced. Also, an expression vector may comprise multiple expression cassettes to increase the amount of copies of the polynucleotide(s) to be transformed.

Another way could be to choose different control sequences for the different polynucleotides, which—depending on the choice—may cause a higher or a lower production of the desired polypeptide(s).

In a second aspect, the present disclosure provides a method of producing a mutant filamentous fungal host cell according to the first aspect of the disclosure comprising the steps of:
a) providing a parent filamentous fungal host cell;
b) modifying the parent filamentous fungal host cell, preferably modifying the genome of the parent filamentous fungal host cell, to yield a mutant filamentous fungal host cell which is deficient, in the cell, in a polypeptide comprising a polypeptide selected from:
   i. a polypeptide with an amino acid sequence according to SEQ ID NO: 5 or a polypeptide with an amino acid sequence at least 70% identical to an amino acid sequence according to SEQ ID NO: 5 which is a functional equivalent of the polypeptide with the amino acid sequence according to SEQ ID NO: 5;
   ii. a polypeptide with an amino acid sequence according to SEQ ID NO: 6 or a polypeptide with an amino acid sequence at least 70% identical to an amino acid sequence according to SEQ ID NO: 6 which is a functional equivalent of the polypeptide with the amino acid sequence according to SEQ ID NO: 6;
   iii. a polypeptide with an amino acid sequence according to SEQ ID NO: 4 or a polypeptide with an amino acid sequence at least 70% identical to an amino acid sequence according to SEQ ID NO: 4 which is a functional equivalent of the polypeptide with the amino acid sequence according to SEQ ID NO: 4;
   iv. a polypeptide encoded by a polynucleotide with a nucleotide sequence according to SEQ ID NO: 2 or 3 or encoded by a polynucleotide at least 70% identical to a nucleotide sequence according to SEQ ID NO: 2 or 3, wherein said polypeptide encoded by a polynucleotide at least 70% identical to a nucleotide sequence according to SEQ ID NO: 2 or 3 is a functional equivalent of the polypeptide encoded by the polynucleotide with a nucleotide sequence according to SEQ ID NO: 2 or 3;
   v. a polypeptide encoded by a polynucleotide capable of hybridising under low, medium or high stringency conditions to the complementary strand of a polynucleotide with a nucleotide sequence according to SEQ ID NO: 2 or 3 or a polypeptide encoded by a polynucleotide capable of hybridizing under low, medium or high stringency conditions to the complementary strand of a polynucleotide with a nucleotide sequence at least 70% identical to a nucleotide sequence according to SEQ ID NO: 2 or 3, wherein said polypeptide encoded by a polynucleotide capable of hybridizing under low, medium or high stringency conditions to the complementary strand of a polynucleotide at least 70% identical to a nucleotide sequence according to SEQ ID NO: 2 or 3 is a functional equivalent of the polypeptide encoded by the polynucleotide with a nucleotide sequence according to SEQ ID NO: 2 or 3;

if compared with a parent filamentous fungal host cell which has not been modified when measured under the same conditions. Preferably the mutant obtained in step b) is a mutant as disclosed in the first aspect. Within this context, it will be clear to those skilled in the art that the specific embodiments and/or choices applicable to the mutant filamentous fungal host cell according to the first aspect of the disclosure may be applicable as well to the other aspects of the disclosure.

In a third aspect, the disclosure provides a method for the production of a compound of interest by microbial fermentation comprising:
a) providing a mutant filamentous fungal host cell according to the first aspect of the disclosure or obtained by a method according to the second aspect of the disclosure capable of expressing the compound of interest,
b) culturing said mutant filamentous fungal host cell under conditions conducive to the expression of the compound of interest,
c) optionally isolating the compound of interest from the culture medium.

In step a) a mutant filamentous fungal host cell may be a mutant host cell as described herein.

In step b) the mutant filamentous fungal host cell of step a) is cultured under conditions conducive to the expression of the compound of interest as described herein. The mutant filamentous fungal host cells are cultivated in a nutrient medium suitable for production of the compound of interest using methods known in the art. For example, the cells may be cultivated by shake flask cultivation, small-scale or large-scale fermentation (including continuous, batch, fed-batch, or solid-state fermentations) in laboratory or industrial fermenters performed in a suitable medium and under conditions allowing the compound of interest to be produced and/or isolated. The cultivation takes place in a suitable nutrient medium comprising carbon and nitrogen sources and additional compounds (such as inorganic salts (e.g. phosphate), trace elements and/or vitamins), using procedures known in the art (see, e. g., Bennett, J. W. and LaSure, L., eds., More Gene Manipulations in Fungi, Academic Press, C A, 1991). Suitable media are available from commercial suppliers or may be prepared using published compositions (e. g., in catalogues of the American Type Culture Collection). The cultivation can be performed under aerobic or anaerobic conditions. Typically, the cultivation will comprise a growth phase mainly directed to formation of biomass and a production phase mainly directed to production of the compound of interest. The growth phase and production phase may overlap to some extent.

In step c) the compound of interest may be optionally isolated. If the compound of interest is secreted into the nutrient medium, the compound can be isolated directly from the medium. If the compound of interest is not secreted, it can be isolated from cell lysates.

The compound of interest as described herein may be isolated by methods known in the art. For example, the compound of interest may be isolated from the nutrient medium by conventional procedures including, but not limited to, centrifugation, filtration, extraction, spray drying, evaporation, or precipitation. The isolated compound of interest may then be further purified by a variety of procedures known in the art including, but not limited to, chromatography (e. g., ion exchange, affinity, hydrophobic, chromatofocusing, and size exclusion), electrophoretic procedures (e.g., preparative isoelectric focusing), differential solubility (e. g., ammonium sulfate precipitation), or extraction (see, e.g., Protein Purification, J.-C. Janson and Lars Ryden, editors, VCH Publishers, New York, 1989). In some applications, the compound of interest may be used without substantial isolation from the culture broth; separation of the culture medium from the biomass may be adequate.

In a preferred embodiment of the method for the production of a compound of interest according to the third aspect, the amount of oxalic acid in the fermentation broth is at least 1% less, at least 5% less, 10% less, at least 20% less, at least 30% less, at least 40% less, preferably at least 50% less, at least 60% less, at least 70% less, at least 80% less, more preferably at least 90% less, at least 91% less, at least 92% less at least 93% less, at least 94% less, most preferably at least 95% less, at least 96% less, at least 97% less, at least 98% less, at least 99% less or 100% less than the amount of oxalic acid in the fermentation broth produced by the parent filamentous fungal host cell when measured under the same conditions.

A mutant filamentous fungal host cell as defined herein may be used in the method for the production of a compound of interest as described herein.

The compound of interest produced in the method for the production of a compound of interest by microbial fermentation may be any compound of interest as described herein.

The disclosure further relates to a compound of interest obtainable by the method of producing a compound of interest according to the third aspect of the disclosure.

The disclosure further relates to a method to reduce the amount of oxalic acid in a filamentous fungal fermentation comprising culturing a mutant filamentous fungal host cell according to the first aspect of the disclosure or obtainable by the method according to the second aspect of the disclosure in a suitable culture medium.

The disclosure further relates to a fermentation broth comprising a compound of interest obtainable by
a) culturing a mutant filamentous fungal host cell according to the first aspect of the disclosure under conditions conducive to the expression of the compound of interest;
b) recovering the fermentation broth comprising the compound of interest.

In the context of the disclosure “fermentation broth”, “culture broth”, or “fermentation liquid” may be used interchangeably. A fermentation broth may comprise culturing medium, the compound of interest and/or the filamentous fungal host cell of the disclosure. Alternatively, a broth may be one from which all cells are absent or substantially absent, for example a supernatant.

Examples of embodiments according to the disclosure

1. A mutant filamentous fungal host cell which has been modified to result in a deficiency in the cell of a polypeptide comprising or being a polypeptide selected from:
a. a polypeptide with an amino acid sequence according to SEQ ID NO: 5 or a polypeptide with an amino acid sequence at least 70% identical to the amino acid sequence according to SEQ ID NO: 5 which is a functional equivalent of the polypeptide with the amino acid sequence according to SEQ ID NO: 5;
b. a polypeptide with an amino acid sequence according to SEQ ID NO: 6 or a polypeptide with an amino acid sequence at least 70% identical to the amino acid sequence according to SEQ ID NO: 6 which is a functional equivalent of the polypeptide with the amino acid sequence according to SEQ ID NO: 6;
c. a polypeptide with an amino acid sequence according to SEQ ID NO: 4 or a polypeptide with an amino acid sequence at least 70% identical to the amino acid sequence according to SEQ ID NO: 4 which is a functional equivalent of the polypeptide with the amino acid sequence according to SEQ ID NO: 4;
d. a polypeptide encoded by a polynucleotide with a nucleotide sequence according to SEQ ID NO: 2 or 3 or encoded by a polynucleotide at least 70% identical to a nucleotide sequence according to SEQ ID NO: 2 or 3, wherein said polypeptide encoded by a polynucleotide at least 70% identical to a nucleotide sequence according to SEQ ID NO: 2 or 3 is a functional equivalent of the polypeptide encoded by the polynucleotide with a nucleotide sequence according to SEQ ID NO: 2 or 3;
e. a polypeptide encoded by a polynucleotide capable of hybridising under low, medium or high stringency conditions to the complementary strand of a polynucleotide with a nucleotide sequence according to SEQ ID NO: 2 or 3 or a polypeptide encoded by a polynucleotide capable of hybridizing under low, medium or high stringency conditions to the complementary strand of a polynucleotide with a nucleotide sequence at least 70% identical to a nucleotide sequence according to SEQ ID NO: 2 or 3, wherein said polypeptide encoded by a polynucleotide capable of hybridizing under low, medium or high stringency conditions to the complementary strand of a polynucleotide at least 70% identical to a nucleotide sequence according to SEQ ID NO: 2 or 3 is a functional equivalent of the polypeptide encoded by the polynucleotide with a nucleotide sequence according to SEQ ID NO: 2 or 3;

if compared with a parent filamentous fungal host cell which has not been modified when measured under the same conditions.

2. The mutant filamentous fungal host cell according to embodiment 1 wherein the polypeptide according to embodiment 1 a. or 1 b. comprises or is a DNA binding domain of a zinc binuclear cluster transcriptional regulator and/or is able to perform the function of a DNA binding domain of a zinc binuclear cluster transcriptional regulator, preferably of a DNA binding domain of a zinc binuclear cluster transcriptional regulator involved in the extracellular production of oxalic acid by a filamentous fungal host cell.

3. The mutant filamentous fungal host cell according to embodiment 1 or 2 wherein the polypeptide according to embodiment 1 c. to 1 e. comprises or is a zinc binuclear cluster transcriptional regulator and/or is able to perform the function of a zinc binuclear cluster transcriptional regulator, preferably a zinc binuclear cluster transcriptional regulator involved in the extracellular production of oxalic acid by a filamentous fungal host cell.

4. The mutant filamentous fungal host cell according to any one of embodiments 1 to 3, wherein the mutant filamentous fungal host cell produces and/or secretes less oxalic acid than the parent filamentous fungal host cell which has not been modified, when cultured and measured under the same conditions, or wherein the mutant filamentous fungal host cell produces extracellularly less oxalic acid than the parent filamentous fungal host cell which has not been modified, when cultured and measured under the same conditions.

5. The mutant filamentous fungal host cell according anyone of embodiments 1 to 4 wherein
a) the polypeptide which has an amino acid sequence according to SEQ ID NO: 5 comprises one or more of
Ala or Ile at position 11, Lys, Asn or Ala at position 12, Gly or Asn at position 15, Gin or His at position 16, Glu, Ser or Ala at position 17, Gly or Arg at position 18, Arg or Gin at position 22, Ile of Leu at position 33, Ser or Cys at position 36, Thr, Ser or Ala at position 38, Pro or Ser at position 40; or
b) the polypeptide which has an amino acid sequence with at least 50% identity to the amino acid sequence according to SEQ ID NO: 5 and which is functionally equivalent to the amino acid sequence according to SEQ ID NO: 5 according to embodiment 1. a. is a polypeptide which, when aligned with the sequence set out in SEQ ID NO: 5, comprises one or more of
   Ala or Ile at position 11, Lys, Asn or Ala at position 12, Gly or Asn at position 15, Gin or His at position 16, Glu, Ser or Ala at position 17, Gly or Arg at position 18, Arg or Gin at position 22, Ile of Leu at position 33, Ser or Cys at position 36, Thr, Ser or Ala at position 38, Pro or Ser at position 40,
   said positions being defined with reference to SEQ ID NO: 5.

6. The mutant filamentous fungal host cell according to embodiment 5 wherein
a) the polypeptide which has an amino acid sequence according to SEQ ID NO: 5 comprises one or more of
Ile at position 11, Lys at position 12, Gly at position 15, Gin at position 16, Glu at position 17, Arg at position 18, Arg at position 22, Leu at position 33, Ser at position 36, Thr at position 38, Pro at position 40; or
b) the polypeptide which has an amino acid sequence according to SEQ ID NO: 5 or the polypeptide which has an amino acid sequence with at least 50% identity to the amino acid sequence according to SEQ ID NO: 5 and which is functionally equivalent to the amino acid sequence according to SEQ ID NO: 5 according to embodiment 1. a. is a polypeptide which, when aligned with the sequence set out in SEQ ID NO: 5, comprises one or more of
   Ile at position 11, Lys at position 12, Gly at position 15, Gin at position 16, Glu at position 17, Arg at position 18, Arg at position 22, Leu at position 33, Ser at position 36, Thr at position 38, Pro at position 40,
   said positions being defined with reference to SEQ ID NO: 5.

7. The mutant filamentous fungal host cell according to any one of embodiments 1 to 6, wherein
a) the polypeptide with an amino acid sequence according to SEQ ID NO: 5 comprises one or more of
an amino acid with a polar uncharged side chain at position 13, an amino acid with an hydrophobic side chain at position 14, an amino acid with a negatively charged side chain at position 26, an amino acid with an hydrophobic side chain at position 37, an amino acid with an hydrophobic side chain at position 46; or
b) the polypeptide which has an amino acid sequence with at least 50% identity to the amino acid sequence according to SEQ ID NO: 5 and which is functionally equivalent to the amino acid sequence according to SEQ ID NO: 5 according to embodiment 1. a. is a polypeptide which, when aligned with the sequence set out in SEQ ID NO: 5, comprises one or more of
   an amino acid with a polar uncharged side chain at position 13, an amino acid with an hydrophobic side chain at position 14, an amino acid with a negatively charged side chain at position 26, an amino acid with an hydrophobic side chain at position 37, an amino acid with an hydrophobic side chain at position 46,
   said positions being defined with reference to SEQ ID NO: 5.

8. The mutant filamentous fungal host cell according to embodiment 7, wherein
a) the polypeptide with an amino acid sequence according to SEQ ID NO: 5 comprises one or more of
Ser, Asn or Thr at position 13, Ala, Phe or Ile at position 14, Asp or Glu at position 26, Leu or Met at position 37, Val, Ala, Leu or Ile at position 46; or
b) the polypeptide which has an amino acid sequence having at least 50% identity to the amino acid sequence according to SEQ ID NO: 5 and which is functionally equivalent to the amino acid sequence according to SEQ ID NO: 5 according to embodiment 1. a. is a polypeptide which, when aligned with the sequence set out in SEQ ID NO: 5, comprises one or more of
Ser, Asn or Thr at position 13, Ala, Phe or Ile at position 14, Asp or Glu at position 26, Leu or Met at position 37, Val, Ala, Leu or Ile at position 46, said positions being defined with reference to SEQ ID NO: 5.

9. The mutant filamentous fungal host cell according to embodiment 7 or 8 wherein the polypeptide which has an amino acid sequence according to SEQ ID NO: 5 or the polypeptide which has an amino acid sequence with at least 50% identity to the amino acid sequence according to SEQ ID NO: 5 according to embodiment 1. a. is a polypeptide comprising an amino acid sequence according to any one of SEQ ID NO: 6, 7, 8, 9, 10, 11, 12, 13, 14, 15, 16, 17, 18, 19, 20, 21, 22, 23 or 24.

10. The mutant filamentous fungal host cell according to any one of embodiments 1 to 9 wherein the polypeptide comprising a polypeptide with an amino acid sequence at least 70% identical to the amino acid sequence according to SEQ ID NO: 4 which is a functional equivalent of the polypeptide with the amino acid sequence according to SEQ ID NO: 4 according to embodiment 1 c. is a polypeptide comprising an amino acid sequence according to any one of SEQ ID NO: 25, 26, 27, 28, or 29.

11. The mutant filamentous fungal host cell according to any one of embodiments 1 to 10 wherein said host cell is deficient in the cell in a polypeptide according to any one of embodiments 1 a. to 1 e., 2, 3, 5 to 10, when
a) it has a modification which results in a reduced amount in the cell of a polypeptide as defined in any one of embodiments 1 a. to 1 e., 2, 3, 5 to 10 or wherein said polypeptide is absent in the cell if compared to the parent filamentous fungal host cell that has not been modified, when measured under the same conditions and/or
b) It has a modification which results in a polypeptide with decreased or no activity if compared to the polypeptide in the parent filamentous fungal host cell that has not been modified, when measured under the same conditions.

12. The mutant filamentous fungal host cell according to any one of embodiments 1 to 11 wherein the modification, preferably a modification in the genome of the host cell, is selected from:
a. a modification which results in a reduced amount or in the absence of a polypeptide as defined in embodiment 1 a. to 1 e. in the cell if compared to the parent filamentous fungal host cell that has not been modified, when analysed under the same conditions and/or
b. a modification which results in a polypeptide with decreased or no activity if compared to the polypeptide in the parent filamentous fungal host cell that has not been modified, when analysed under the same conditions.

13. The mutant filamentous fungal host cell according to any one of embodiments 1 to 12, wherein the mutant filamentous fungal host cell produces 1% less polypeptide as defined in embodiment 1 a. to 1 e. if compared with the parent filamentous fungal host cell which has not been modified and measured under the same conditions, at least 5% less, at least 10% less, at least 20% less, at least 30% less, at least 40% less, at least 50% less, at least 60% less, at least 70% less, at least 80% less, at least 90% less, at least 91% less, at least 92% less, at least 93% less, at least 94% less at least 95% less, at least 96% less, at least 97% less, at least 98% less, at least 99% less, or at least 99.9% less, preferably the mutant filamentous fungal host cell produces substantially no polypeptide as defined in embodiment 1 a. to 1 d. if compared with the parent filamentous fungal host cell which has not been modified when measured under the same conditions.

14. The mutant filamentous fungal host cell according to any one of embodiments 1 to 13, wherein the mutant microbial host cell produces a polypeptide derived from the polypeptide as defined in embodiment 1 a. to 1 e. with 1% less activity, if compared with the parent filamentous fungal host cell which has not been modified and measured under the same conditions, at least 5% less activity, at least 10% less activity, at least 20% less activity, at least 30% less activity, at least 40% less activity, at least 50% less activity, at least 60% less activity, at least 70% less activity, at least 80% less activity, at least 90% less activity, at least 91% less activity, at least 92% less activity, at least 93% less activity, at least 94% less activity, at least 95% less activity, at least 96% less activity, at least 97% less activity, at least 98% less activity, at least 99% less activity, or at least 99.9% less activity, preferably the mutant filamentous fungal host cell produces a polypeptide derived from a polypeptide as defined in embodiment 1 a. to 1 d. with substantially no activity if compared with the parent filamentous fungal host cell which has not been modified when measured under the same conditions.

15. The mutant filamentous fungal host cell according to any one of embodiments 1 to 14, wherein the modification in its genome is selected from:
i. an insertion, deletion, or replacement of one or more nucleotides in a polynucleotide sequence coding for the polypeptide according to any one of embodiment 1 a. to 1 e. or in a regulatory element required for transcription or translation of said polynucleotide sequence, to result in the reduced or no expression in the cell of a polypeptide according to any one of embodiment 1 a. to 1 e.;
ii. an insertion, deletion, or replacement of one or more nucleotides in the polynucleotide sequence coding for the polypeptide according to embodiment 1 a. to 1 e. or in a regulatory element required for transcription of translation of said polynucleotide sequence, to result in the expression in the cell of a polypeptide which is less active or has not activity if compared to the polypeptide according to any one of embodiment 1 a. to 1 e.

16. The mutant filamentous fungal host cell according to any one of embodiments 1 to 14 wherein the mutant comprises a modification in its genome selected from:
a) a full or partial deletion of a polynucleotide encoding for a polypeptide according to any one of embodiments 1 a. to 1 e., 2, 3, 5-10,
b) a full or partial replacement of a polynucleotide encoding for a polypeptide according to any one of embodiments 1 a. to 1 e., 2, 3, 5-10 with a polynucleotide sequence which does not code for said polypeptide or which code for a partially or fully inactive form of said polypeptide
c) a disruption of a polynucleotide according to any one of embodiments 1 a. to 1 e., 2, 3, 5-10 by the insertion of one or more nucleotides in the polynucleotide sequence.

17. The mutant filamentous fungal host cell according to any one of embodiments 1 to 16, wherein the modification results in a reduced amount in the cell of (functional) mRNA encoding for a polypeptide according to any one of embodiments 1 a. to 1 e., if compared to the amount in the parent filamentous fungal cell which has not been modified, when measured under the same conditions.

18. The mutant filamentous fungal host cell according to any one of embodiments 1 to 17 comprising at least one polynucleotide coding for a compound of interest or at least one polynucleotide coding for a compound involved in the production of a compound of interest.

19. The mutant filamentous fungal host cell according to embodiment 18 wherein the at least one polynucleotide coding for the compound of interest or the at least one polynucleotide coding for a compound involved in the production of a compound of interest is operably linked to a promoter capable to promote the expression of said polypeptide in the host cell.

20. The mutant filamentous fungal host cell according to any one of embodiments 1 to 18 which is a filamentous fungus selected from Aspergillus, Acremonium, Myceliophthora, Thielavia Chrysosporium, Neurospora, Penicillium, Talaromyces, Rasamsonia, Fusarium, Humicola or Trichoderma, preferably a species of Aspergillus niger, Aspergillus awamori, Aspergillus flavus, Aspergillus bombycis, Aspergillus calidoustus, Aspergillus ochraceoroseus, Aspergillus nomius, Aspergillus aculeatus, Aspergillus carbonarius, Aspergillus parasiticus, Aspergillus kawachii, Aspergillus luchuensis, Aspergillus brasiliensis, Aspergillus foetidus, Aspergillus sojae, Aspergillus fumigatus, Aspergillus oryzae, Aspergillus nidulans, Aspergillus japonicus, Acremonium alabamense, Myceliophthora thermophila, Thielavia terrestris, Chrysosporium lucknowense, Chrysosporium inops, Chrysosporium keratinophilum, Chrysosporium merdarium, Chrysosporium pannicola, Chrysosporium queenslandicum, Chrysosporium tropicum, Chrysosporium zonatum, Fusarium oxysporum, Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Rasamsonia emersonii, Talaromyces emersonii, Trichoderma reesei, Trichoderma harzianum, Trichoderma koningii, Trichoderma longibrachiatum, Trichoderma viridae, Penicillium brasilianum, Penicillium camemberti, Penicillium rubens or Penicillium chrysogenum.

21. A method of producing a mutant filamentous fungal host cell according to any one of embodiments 1 to 20 comprising the steps of:
a) providing a parent filamentous fungal host cell;
b) modifying the parent filamentous fungal host cell to yield a mutant filamentous fungal host cell which is deficient in the cell in a polypeptide comprising or being a polypeptide selected from:
   i. a polypeptide with an amino acid sequence according to SEQ ID NO: 5 or a polypeptide with an amino acid sequence at least 70% identical to the amino acid sequence according to SEQ ID NO: 5 which is a functional equivalent of the polypeptide with the amino acid sequence according to SEQ ID NO: 5;
   ii. a polypeptide with an amino acid sequence according to SEQ ID NO: 6 or a polypeptide with an amino acid sequence at least 70% identical to the amino acid sequence according to SEQ ID NO: 6 which is a functional equivalent of the polypeptide with the amino acid sequence according to SEQ ID NO: 6;
   iii. a polypeptide with an amino acid sequence according to SEQ ID NO: 4 or a polypeptide with an amino acid sequence at least 70% identical to the amino acid sequence according to SEQ ID NO: 4 which is a functional equivalent of the polypeptide with the amino acid sequence according to SEQ ID NO: 4;
   iv. a polypeptide encoded by a polynucleotide with a nucleotide sequence according to SEQ ID NO: 2 or 3 or encoded by a polynucleotide at least 70% identical to a nucleotide sequence according to SEQ ID NO: 2 or 3, wherein said polypeptide encoded by a polynucleotide at least 70% identical to a nucleotide sequence according to SEQ ID NO: 2 or 3 is a functional equivalent of the polypeptide encoded by the polynucleotide with a nucleotide sequence according to SEQ ID NO: 2 or 3;
   v. a polypeptide encoded by a polynucleotide capable of hybridising under low, medium or high stringency conditions to the complementary strand of a polynucleotide with a nucleotide sequence according to SEQ ID NO: 2 or 3 or a polypeptide encoded by a polynucleotide capable of hybridizing under low, medium or high stringency conditions to the complementary strand of a polynucleotide with a nucleotide sequence at least 70% identical to a nucleotide sequence according to SEQ ID NO: 2 or 3, wherein said polypeptide encoded by a polynucleotide capable of hybridizing under low, medium or high stringency conditions to the complementary strand of a polynucleotide at least 70% identical to a nucleotide sequence according to SEQ ID NO: 2 or 3 is a functional equivalent of the polypeptide encoded by the polynucleotide with a nucleotide sequence according to SEQ ID NO: 2 or 3;
if compared with a parent filamentous fungal host cell which has not been modified when measured under the same conditions.

22. The method according to embodiment 21 wherein the mutant obtained in step b) is a mutant according to any one of embodiments 1 to 20.

23. A method for the production of a compound of interest by microbial fermentation comprising:
a) providing a mutant filamentous fungal host cell according to any one of embodiments 1 to 20 or obtained by a method according to embodiment 21 or 22, capable of expressing the compound of interest,
b) culturing said mutant filamentous fungal host cell under conditions conducive to the expression of the compound of interest,
c) optionally isolating the compound of interest from the culture medium.

24. The method according to embodiment 23 wherein the compound of interest is a biological compound selected from the group consisting of biomass, a biopolymer, a metabolite, preferably the compound of interest is selected from a biopolymer or a primary or secondary metabolite.

25. The method according to embodiment 24 wherein the biopolymer is selected from a nucleic acid, a polyamine, a polyol, a polypeptide (such as a protein, preferably an enzyme), a polyamide, a polysaccharide, more preferably wherein the biopolymer is an enzyme, even more preferably wherein the compound of interest is an enzyme.

26. The method according to any one of embodiments 21 to 25 wherein the amount of oxalic acid in the fermentation broth is at least 1% less, at least 5% less, 10% less, at least 20% less, at least 30% less, at least 40% less, preferably at least 50% less, at least 60% less, at least 70% less, at least 80% less, more preferably at least 90% less, at least 91% less, at least 92% less at least 93% less, at least 94% less, most preferably at least 95% less, at least 96% less, at least 97% less, at least 98% less, at least 99% less or 100% less than the amount of oxalic acid in the fermentation broth produced by the parent filamentous fungal host cell when cultured and measured under the same conditions.

27. A compound of interest obtainable by the method according to any one of embodiments 23 to 26.

28. A method to reduce the amount of oxalic acid in a filamentous fungal fermentation comprising culturing a mutant filamentous fungal host cell according to any one of embodiments 1 to 20 or obtainable by the method according to embodiments 21 or 22 in a suitable culture medium.

29. A fermentation broth comprising a compound of interest obtainable by
a) culturing a mutant filamentous fungal host cell according to any one of embodiments 1 to 20 or obtainable by the method according to embodiments 21 or 22 under conditions conducive to the expression of the compound of interest;
b) recovering the fermentation broth comprising the compound of interest.

### Example 1

Strains

WT 1: Aspergillus niger CBS 513.88 strain is used as a wild-type strain. This strain, is available at the Fungal Genetics Stock Center (Manhattan, Kansas, USA) under the access number A1513 (McCluskey et al, J. Biosci. (2010) 35(1): 119-126). Aspergillus niger CBS 513.88 has been deposited by Gist Brocades (now DSM) with the Centraal Bureau voor Schimmelculturen (Utrecht, the Netherlands) on 10 Aug. 1988, an institute which is now renamed as Westerdijk Fungal Biodiversity Institute. A. niger CBS 513.88 is derived from A. niger NRRL 3122 which was deposited in 1964 by a researcher from the Fermentation Research Institute in Ministry of International Trade and Industry located in Chiba, Japan. The history of the strain prior to the deposit is not readily available. (Baker Medical Mycology (2006) 44: S17-S21). A. niger NRRL3122 was acquired from the Culture Collection Unit of the Northern Utilization Research and Development Division, US Department of Agriculture, Peoria, Ill., USA. The strain NRRL3122 and its classical derivatives have been in use to produce glucoamylase (and acid amylase) by Wallerstein Laboratories (USA) since the sixties. Wallerstein division was part of Baxter-Travenol Laboratories (USA) but was divested in 1977 to Gist-Brocades (the Netherlands), now part of DSM (van Dijck et al Regulatory Toxicology and Pharmacology (2003) 38: 27-35).

GBA 306: The construction of GBA 306 using WT1 as starting strain has been described in detail in WO2011/009700. This GBA 306 strain has the following genotype: ΔglaA, ΔpepA, ΔhdfA, an adapted BamHI amplicon, ΔamyBII, ΔamyBI, and ΔamyA.

GBA 307: The construction of GBA 307 using GBA306 as starting strain has been described in detail in WO2011/009700. This GBA 307 strain has the following genotype: ΔglaA, ΔpepA, ΔhdfA, an adapted BamHI amplicon, ΔamyBII, ΔamyBI, ΔamyA and ΔoahA).

PEP1: This A. niger strain is a GBA306-derived strain expressing the proline-specific endoprotease (PEP) of Rasamsonia emersonii. The protein sequence of the proline-specific endoprotease (PEP) of R. emersonii is shown in SEQ ID NO: 1 and is also called PEP BC2G079 (previously described in WO2015177171, example 1, as SEQ ID NO: 2). The detailed construction of the pGBTOP-PEP expression vector, the co-transformation of GBA306 and subsequent selection and purification, resulting in strain PEP1 is fully described in WO2015177171, Example 1.

Molecular Biology Techniques

In these strains, using molecular biology techniques known to the skilled person (see: Sambrook & Russell, Molecular Cloning: A Laboratory Manual, 3rd Ed., CSHL Press, Cold Spring Harbor, N.Y., 2001), several genes were over expressed and others were down regulated as described below. Examples of the general design of expression vectors for gene over expression and disruption vectors for down-regulation, transformation, use of markers and selective media can be found in WO199846772, WO199932617, WO2001121779, WO2005095624, WO2006040312, EP635574B, WO2005100573, WO2011009700 and WO2012001169. In general gene replacement vectors comprise approximately 0.5-2 kb flanking regions of the respective ORF sequences, to target for homologous recombination at the predestined genomic loci. In addition, they contain a selectable marker for transformation, such as an A. nidulans bi-directional amdS selection marker, or a hygromycin- or phleomycin-resistance marker cassette for example, in-between direct repeats. The method applied for gene deletion in all examples herein uses linear DNA, which integrates into the genome at the homologous locus of the flanking sequences by a double cross-over, thus substituting the gene to be deleted by the marker gene.

All nucleotide and amino acid sequences for A. niger and many other fungal genes, their genomic context and proteins encoded can be derived for example from NCBI (http://www.ncbi.nlm.nih.gov/) or EMBL (http://www.ebi.ac.uk.embl/) or AspGD (http://www.aspergillusgenome.org/).

A. niger Shake Flask Fermentations

A. niger strains were pre-cultured in 20 ml CSL pre-culture medium (100 ml flask, baffle) as described in the Examples: “Aspergillus niger shake flask fermentations” section of WO 99/32617. After growth for 18-24 hours at 34° C. and 170 rpm, 10 ml of this culture is transferred to Fermentation Medium (FM). Fermentation in FM is performed in 500 ml flasks with baffle with 100 ml FM at 34° C. and 170 rpm for the number of days indicated, generally as described in WO99/32617.

The CSL medium consisted of (in amount per liter): 100 g Corn Steep Solids (Roquette), 1 g NaH2PO4*H2O, 0.5 g MgSO4*7H2O, 10 g glucose*H2O and 0.25 g Basildon (antifoam). The ingredients were dissolved in demi-water and the pH was adjusted to pH5.8 with NaOH or H2SO4; 100 ml flasks with baffle and foam ball were filled with 20 ml CSL pre-culture medium and sterilized for 20 minutes at 120° C.

The fermentation medium (FM) consisted of (in amount per liter): 150 g maltose*H2O, 60 g Soytone (peptone), 1 g NaH2PO4*H2O, 15 g MgSO4*7H2O, 0.08 g Tween 80, 0.02 g Basildon (antifoam), 20 g MES, 1 g L-arginine. The ingredients were dissolved in demi-water and the pH was adjusted to pH 6.2 with NaOH or H2SO4; 500 ml flasks with baffle and foam ball were filled with 100 ml FM and sterilized for 20 minutes at 120° C.

At the end of the fermentation, samples of the fermentation broth were centrifuged at 5000 g for 10 minutes and the supernatants were collected for subsequent analyses.

Enzyme Activity Measurements

Proline-specific endoprotease (PEP) activity measurements were performed as described in example 2 of WO2015177171 on the supernatants collected after fermentation. The activity is expressed in pNASU's. 1 pNASU is the amount of enzyme which liberates from Ac-AAP-pNA in 1 hour the amount of pNA that corresponds to an increase in absorption at 405 nm of 1 OD, using the conditions as described in WO2015177171, Example 2.

Assay for the Determination of Oxalic Acid in Fermentation Broth

Preparation of the Samples

Oxalic acid (Sigma) was used as an external standard. The external standard sample was prepared by mixing 1 part of a 3.3 N HClO4 and 10 parts of a 0.20 mg/L solution of oxalic acid in a 1:10 ratio. The obtained solution was used as such.

The fermentation broth supernatant obtained after shake-flask fermentation experiment was pre-treated by mixing 10 parts of the supernatant and 1 part of a 3.3 N HClO4, solution, keeping in refrigerator for 1 hour and centrifuging. The obtained supernatant was diluted up to an expected concentration of oxalic acid in the sample between 0.1-30 g/l.

Determination of Oxalic Acid Using a Phenomenex® Rezex™ RHM-Monosaccharide Column Based on Size Exclusion, Ion-Exclusion and Ion Exchange Using Reversed Phase Mechanisms.

20 micro liters of pre-treated sample or of standard solution were injected on a Phenomenex® Rezex™ RHM-Monosaccharide column (diameter 7.8 mm, length of 300 mm) (column temperature of 50° C.), and elution was performed at a flow of 0.6 ml/min during 27 minutes, using 0.01 N H₂SO₄ as eluent, and UV detection at 214 nm.

The amount of oxalic acid in the sample was measured from the area of the elution peak and compared to the external standard measured under the same conditions.

### Example 1

Construction of an Aspergillus Niger PEP1 ORER-1 Strain

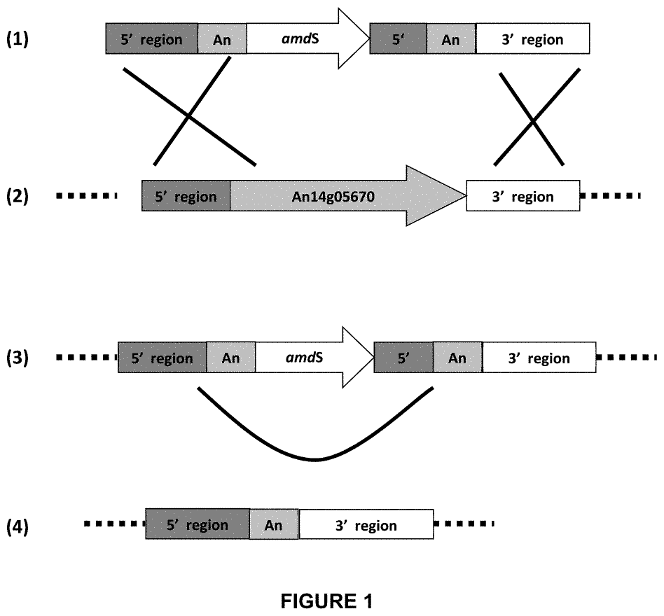

To be able to disrupt the OreR oxalate C6 transcriptional regulator, encoded by the An14g05670 (oreR) gene (also called ANI_1_780124, with a genomic sequence as depicted in SEQ ID NO: 2, a coding sequence as depicted in SEQ ID NO: 3, and an OreR protein (also called XP_001401190.2) encoded as depicted in SEQ ID NO: 4, a gene replacement vector was designed as described above (FIG. 1).

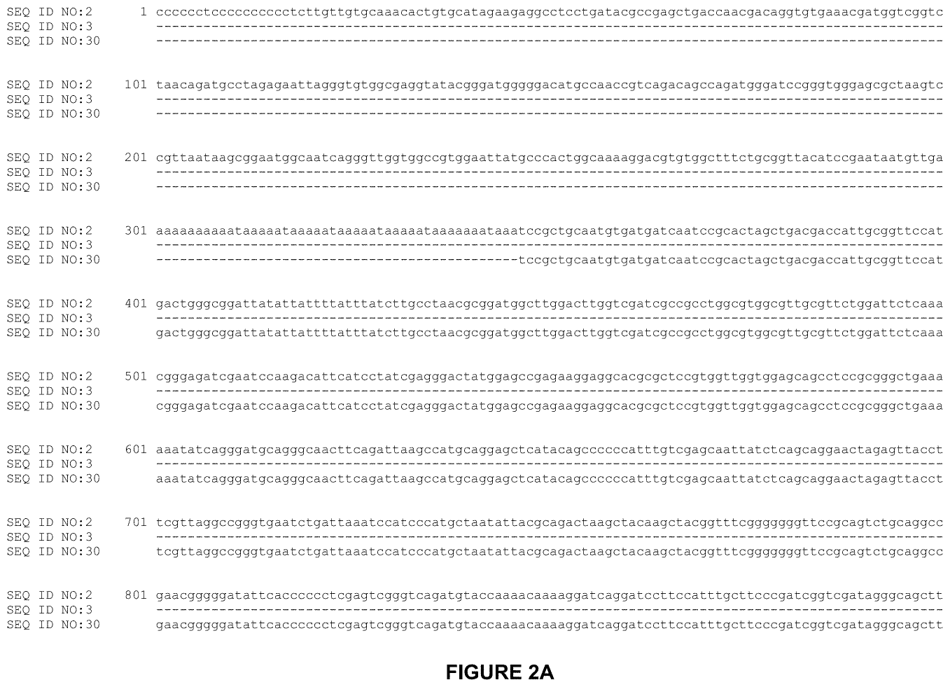

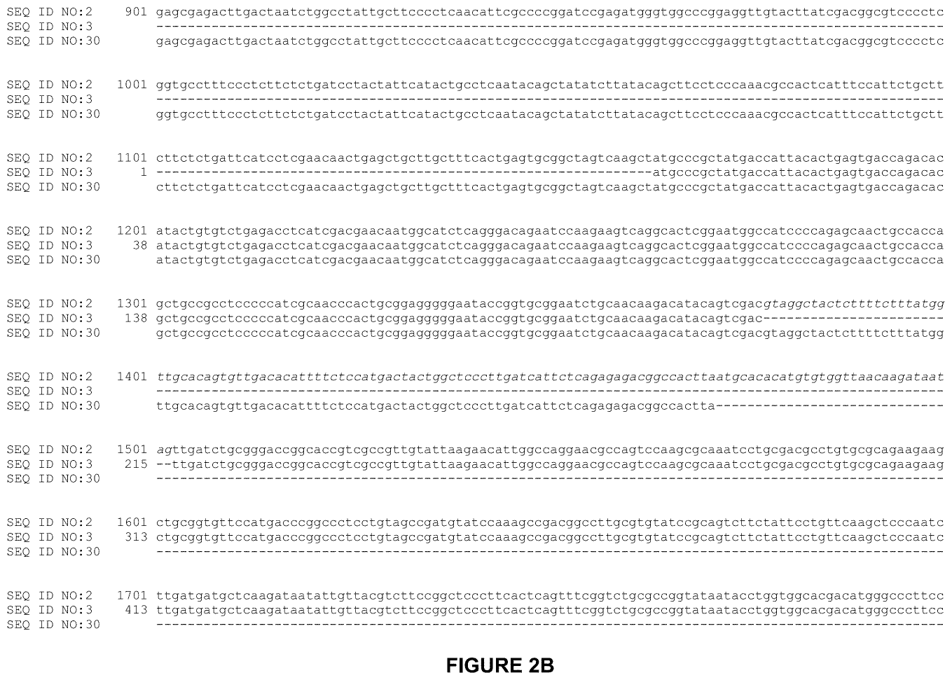

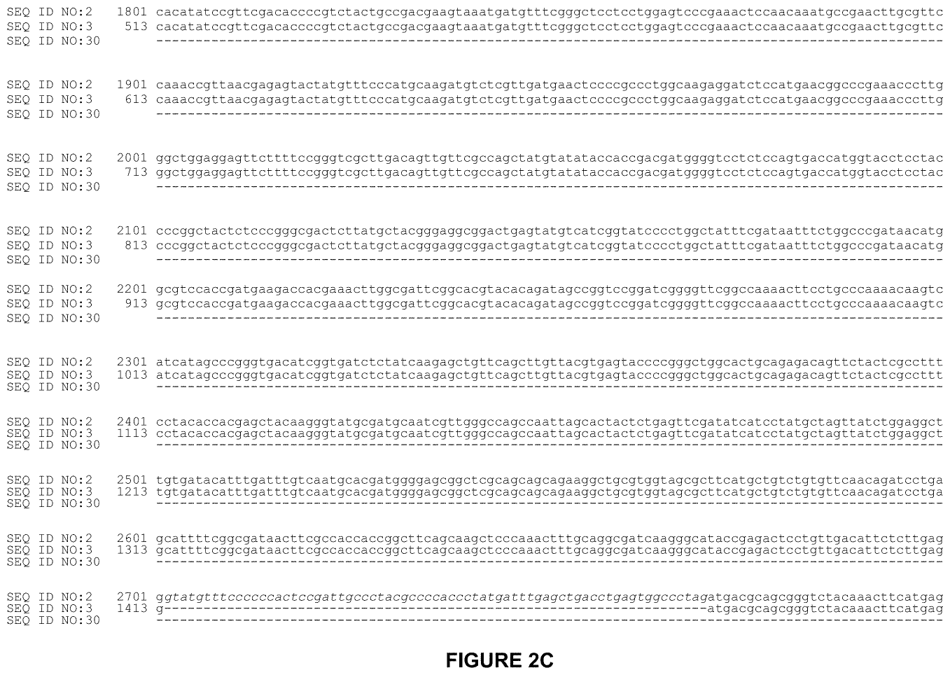

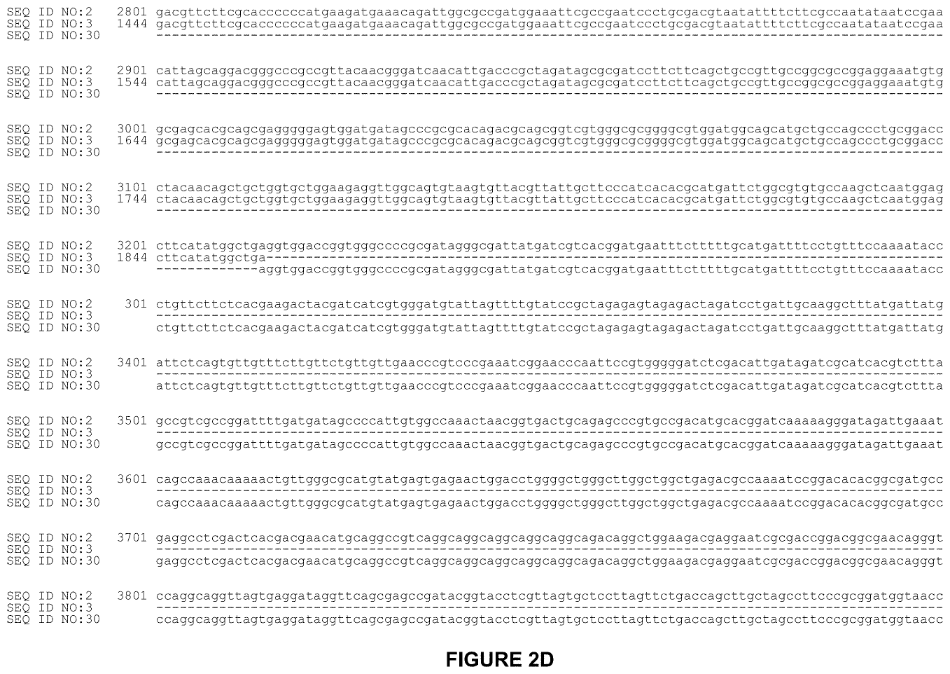

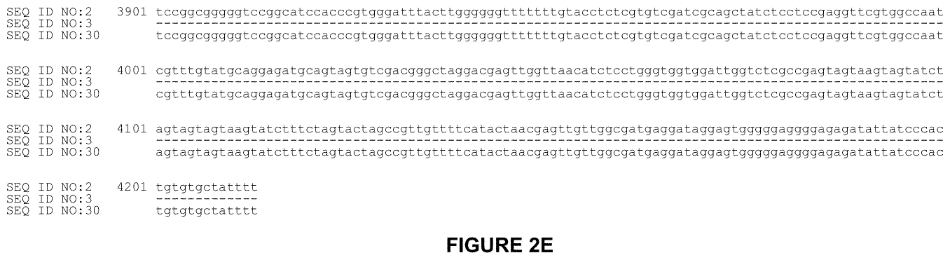

In FIG. 2, an alignment of the genomic sequence (“Genomic DNA”, SEQ ID NO: 2) with the oreR coding sequence (“CDS”, SEQ ID NO: 3) and the sequence of the disrupted oreR locus (“Disruption seq”) is shown. From this FIG. 2, the oreR gene, including 2 introns therein, can be deduced. Vector pGBDEL-ORER was constructed by DNA synthesis and comprised approximately 1 kb flanking regions comprising the oreR promoter (5′ oreR flanking region including approximately 300 bp of the 5′-end of the oreR gene), and oreR terminator region (3′ oreR flanking region) of oreR as targeting regions for homologous recombination. The general procedure for gene disruption is depicted in FIG. 1. The pGBDEL-ORER vector was linearized and used to transform the Aspergillus niger PEP1 strain. After selection of a correct transformant with an integration of the linear fragment at the oreR locus, four PEP1_ORER strains (numbered PEP1_ORER1-4) were selected as representative strains with the oreR gene inactivated in the PEP1 strain background.

### Example 2

Analysis of the A. niger PEP1 ORER Strains

To be able to assess the effect of the oreR gene disruptions, shake-flask analysis was performed. After a preculture in CSL-medium as described above, part of the culture was transferred into FM medium for the PEP1 and PEP1-derived transformants as detailed in Table 1. At day 5 after inoculation, medium samples were taken. The proline-specific endoprotease (PEP) activity levels were analysed in the culture supernatant using the assay described above together with the oxalic acid amount using the assay described in WO2015177171, Example 2. Surprisingly, a disruption of oreR—(An14g05670—ANI_1_780124) resulted in a drastic reduction of oxalic acid production, as detailed in Table 1. This decreased oxalate production upon oreR disruption is paralleled by a good enzyme productivity (Table 1). These results clearly show that a filamentous fungal cell with an oreR disruption has a decreased oxalic acid production. Furthermore, these results clearly show that a filamentous fungal host cell with an oreR disruption has a decreased oxalic acid production which is comparable to a filamentous fungal host cell deficient in the gene oahA coding for the enzyme oxaloacetate hydrolase according to the prior art (strain GBA307).

TABLE 1
Relative oxalic acid production and proline-
specific endoprotease levels in the supernatant of
PEP1, GBA306, GBA307 and PEP1_ORER1-4

| | | Proline-specific |
|---|---|---|
| Strain | Oxalic Acid | endoprotease(PEP) activity |
| GBA306 | 100% | N.A. |
| GBA307 | ~1% | N.A. |
| PEP1 | 100% | 100% |
| PEP1_ORER1 | ~1% | 100% |
| PEP1_ORER2 | ~1% | 100% |
| PEP1_ORER3 | ~1% | 100% |
| PEP1_ORER4 | ~1% | 100% |

### Example 3

Identification of GAL4-Like Zn(II)₂Cys₆ (or C6 Zinc) Binuclear Cluster DNA-Binding Proteins with Highly Similar DNA Binding Domain.

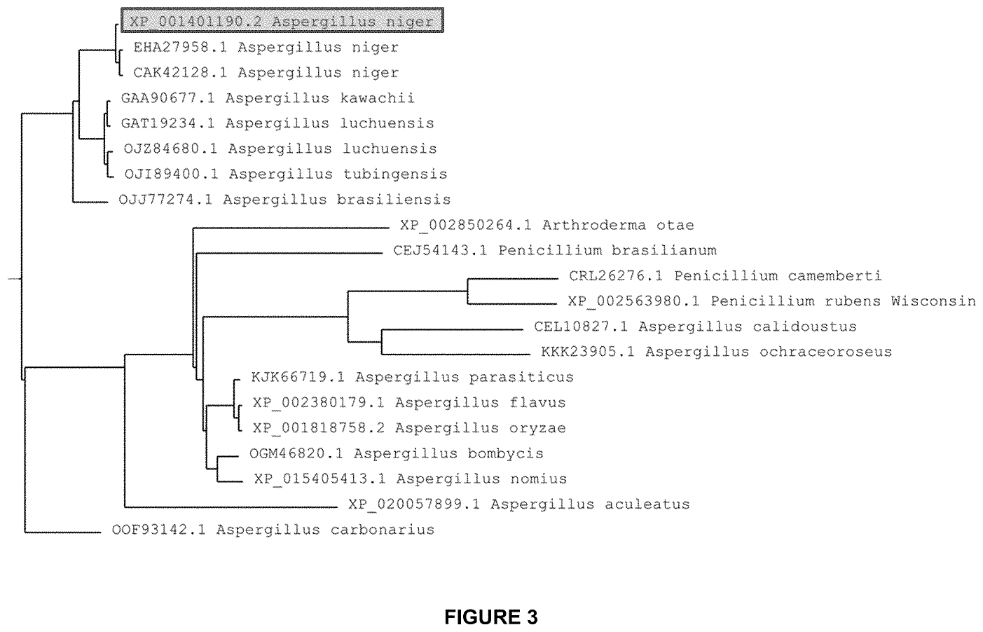

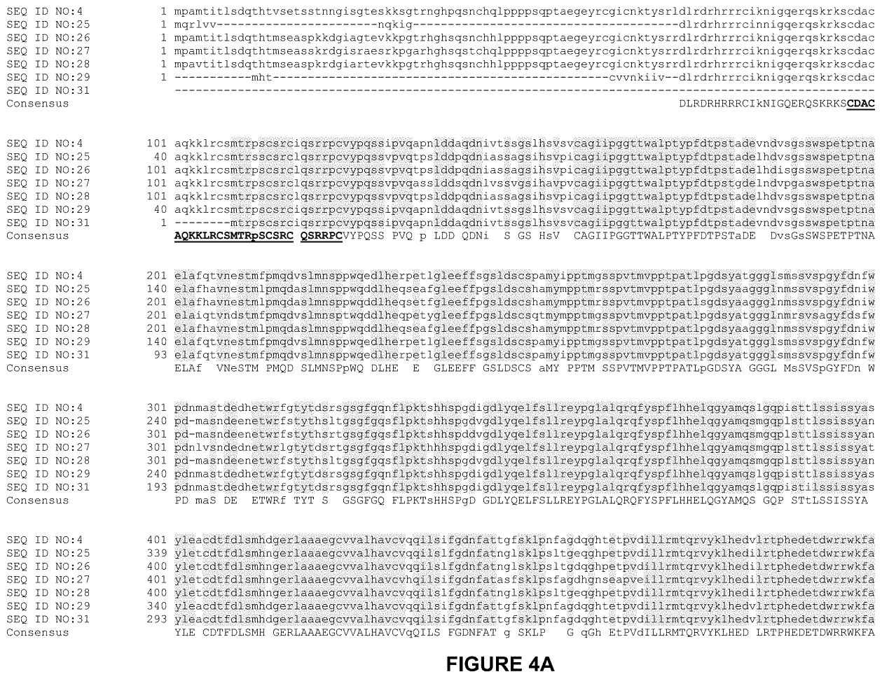

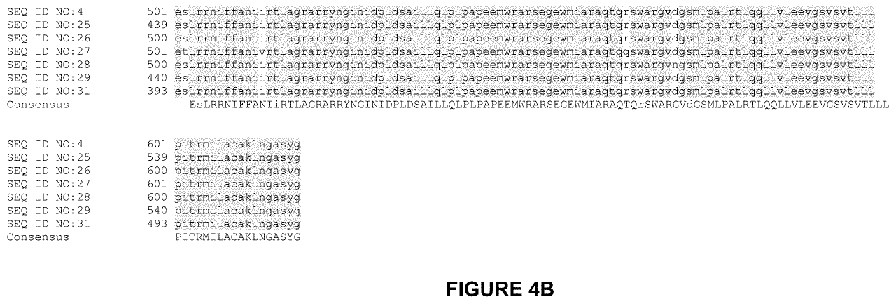

In this example, we show how the A. niger OreR protein sequence (SEQ ID NO 4) can be used to identify functional homologues in other organisms. OreR orthologues were identified in other filamentous fungi by phylogenetic identification using Blosum 62 matrix available in Clone Manager Professional, version 9.4 for Windows (© 2015 Scientific & Educational Software) and are depicted in FIG. 3. FIG. 3 clearly shows that several highly-related transcriptional regulators can be identified in various Aspergillus species, along with several somewhat more distantly related transcription factors in filamentous fungi. In FIG. 4, the alignment of the full OreA amino acid sequence with the most closely related orthologous transcriptional regulators is shown. Clearly a high overall identity is shown for the different transcription factors listed. Observing the alignment in FIG. 4, it can be observed that the sequence of EHA27958.1 is deviant if compared to the sequence of all the other putative zinc binuclear transcriptional regulators, missing the first 108 amino acid when aligned with SEQ ID NO: 4, as it is almost fully missing the putative DNA binding domain. The protein sequence of EHA27958.1 is however identical to the sequence of oreR starting from amino acid 109. Since EHA27958.1 is also a protein from A. niger (Aspergillus niger ATCC 1015) it is expected that the reason of this difference is due to either a mistake in the annotation of the corresponding gene or to a gene which became inactive in this species.

In FIG. 5, a homology search using the DNA binding domain of the OreR amino acid sequence was performed. In FIG. 5, an amino acid consensus sequence for the DNA binding domain can be deduced for a large set of orthologous transcription factors within the alignment.

The consensus sequence with the 6 cysteines indicated in bold is:
DLRDRHRRRCikXXgqerXSKrKSCXXCAQKKIRCsXtRpXCXRCXQXXXXCXYP from which the following fully conserved sequence can be deduced:

(SEQ ID NO: 5)
DLRDRHRRRCXXXXXXXXXSKXKSCXXCAQKKXRCXXXRXXCXRCXQX
XXXCXYP

(wherein X may be any natural amino acid).

Many transcription factors share a high identity in this DNA binding domain, suggesting a functional similarity. All proteins listed are available in the sequence databases accessible for instance via the National Centrum for Biotechnology Information (NCBI https://www.ncbi.nlm.nih.gov/), EMBL (http://www.ebi.ac.uk/embl/) or DNA Database of Japan (DDBJ—http://www.ddbj.nig.ac.jp/).

1. A mutant filamentous fungal host cell which has been modified, if compared with a parent filamentous fungal host cell which has not been modified, when measured under the same conditions, to result in a deficiency in the cell of a polypeptide comprising a polypeptide selected from:
a. a polypeptide with an amino acid sequence according to SEQ ID NO: 5;
b. a polypeptide with an amino acid sequence according to SEQ ID NO: 6 or a polypeptide with an amino acid sequence at least 70% identical to the amino acid sequence according to SEQ ID NO: 6 which is a functional equivalent of the polypeptide with the amino acid sequence according to SEQ ID NO: 6, wherein said polypeptide with an amino acid sequence according to SEQ ID NO: 6 or said polypeptide with an amino acid sequence at least 70% identical thereto optionally comprises or is a DNA binding domain of a zinc binuclear cluster transcriptional regulator and/or is able to perform the function of a DNA binding domain of a zinc binuclear cluster transcriptional regulator;
c. a polypeptide with an amino acid sequence according to SEQ ID NO: 4 or a polypeptide with an amino acid sequence at least 70% identical to the amino acid sequence according to SEQ ID NO: 4 which is a functional equivalent of the polypeptide with the amino acid sequence according to SEQ ID NO: 4, wherein said polypeptide with an amino acid sequence according to SEQ ID NO: 4 or said polypeptide with an amino acid sequence at least 70% identical thereto optionally comprises or is a zinc binuclear cluster transcriptional regulator and/or is able to perform the function of a zinc binuclear cluster transcriptional regulator;
wherein the mutant filamentous fungal host cell produces extracellularly less oxalic acid than the parent filamentous fungal host cell which has not been modified, when cultured and measured under the same conditions.

2. The mutant filamentous fungal host cell according to claim 1 wherein the polypeptide a or b comprises or is a DNA binding domain of a zinc binuclear cluster transcriptional regulator and/or is able to perform the function of a DNA binding domain of a zinc binuclear cluster transcriptional regulator.

3. The mutant filamentous fungal host cell according to claim 1 wherein the polypeptide c. comprises or is a zinc binuclear cluster transcriptional regulator and/or is able to perform the function of a zinc binuclear cluster transcriptional regulator.

4. The mutant filamentous fungal host cell according to claim 1 wherein the mutant host cell is deficient in the polypeptide a to c according when the host cell
a) has a modification which results in a reduced amount in the cell of the polypeptide a to c or wherein said polypeptide is absent in the cell if compared to the parent filamentous fungal host cell that has not been modified, when measured under the same conditions and/or
b) has a modification which results in a polypeptide with decreased or no activity if compared to the polypeptide in the parent filamentous fungal host cell that has not been modified, when measured under the same conditions.

5. The mutant filamentous fungal host cell according to claim 1 wherein the modification in the mutant host cell is a modification in the genome thereof is selected from:
a) an insertion, deletion, or replacement of one or more nucleotides in a polynucleotide sequence coding for the polypeptide a to c or in a regulatory element required for transcription or translation of said polynucleotide sequence, to result in the reduced or no expression in the cell of a polypeptide a to c;
b) an insertion, deletion, or replacement of one or more nucleotides in the polynucleotide sequence coding for the polypeptide a to c or in a regulatory element required for transcription or translation of said polynucleotide sequence, to result in the expression in the cell of a polypeptide which is less active or has no activity if compared to the polypeptide a to c;

6. The mutant filamentous fungal host cell according to claim 1 wherein the modification results in a reduced amount in the cell of (functional) mRNA encoding for a polypeptide a to c., if compared to the amount in the parent filamentous fungal host cell which has not been modified, when measured under the same conditions.

7. The mutant filamentous fungal host cell according to claim 1 wherein the polypeptide which has an amino acid sequence according to SEQ ID NO: 5 comprises one or more of
Ala or Ile at position 11, Lys, Asn or Ala at position 12, Gly or Asn at position 15, Gin or His at position 16, Glu, Ser or Ala at position 17, Gly or Arg at position 18, Arg or Gin at position 22, Ile of Leu at position 33, Ser or Cys at position 36, Thr, Ser or Ala at position 38, Pro or Ser at position 40.

8. The mutant filamentous fungal host cell according to claim 7 wherein the polypeptide which has an amino acid sequence according to SEQ ID NO: 5 comprises one or more of
Ile at position 11, Lys at position 12, Gly at position 15, Gin at position 16, Glu at position 17, Arg at position 18, Arg at position 22, Leu at position 33, Ser at position 36, Thr at position 38, Pro at position 40.

9. The mutant filamentous fungal host cell according to claim 1 wherein the polypeptide with an amino acid sequence according to SEQ ID NO: 5 comprises one or more of
an amino acid with a polar uncharged side chain at position 13, an amino acid with an hydrophobic side chain at position 14, an amino acid with a negatively charged side chain at position 26, an amino acid with an hydrophobic side chain at position 37, an amino acid with an hydrophobic side chain at position 46.

10. The mutant filamentous fungal host cell according to claim 9, wherein the polypeptide with an amino acid sequence according to SEQ ID NO: 5 comprises one or more of
Ser, Asn or Thr at position 13, Ala, Phe or Ile at position 14, Asp or Glu at position 26, Leu or Met at position 37, Val, Ala, Leu or Ile at position 46.

11. The mutant filamentous fungal host cell according to claim 9 wherein the polypeptide which has an amino acid sequence according to SEQ ID NO: 5 is a polypeptide comprising an amino acid sequence according to any one of SEQ ID NO: 6, 7, 8, 9, 10, 11, 12, 13, 14, 15, 16, 17, 18, 19, 20, 21, 22, 23 or 24.

12. The mutant filamentous fungal host cell according to claim 1 wherein the polypeptide comprising a polypeptide with an amino acid sequence at least 70% identical to the amino acid sequence according to SEQ ID NO: 4 which is a functional equivalent of the polypeptide with the amino acid sequence according to SEQ ID NO: 4 of c is a polypeptide comprising an amino acid sequence according to any one of SEQ ID NO: 25, 26, 27, 28, or 29.

13. The mutant filamentous fungal host cell according to claim 1 comprising at least one polynucleotide coding for a compound of interest or at least one polynucleotide coding for a compound involved in the production of a compound of interest.

14. The mutant filamentous fungal host cell according to claim 13 wherein the at least one polynucleotide coding for the compound of interest or the at least one polynucleotide coding for a compound involved in the production of a compound of interest is operably linked to a promoter capable to promote the expression of said polypeptide in the host cell.

15. The mutant filamentous fungal host cell according to claim 1 which is a filamentous fungus selected from Aspergillus, Acremonium, Myceliophthora, Thielavia Chrysosporium, Neurospora, Penicillium, Talaromyces, Rasamsonia, Fusarium, Humicola or Trichoderma, optionally a species of Aspergillus, more optionally an Aspergillus niger, Aspergillus awamori, Aspergillus flavus, Aspergillus bombycis, Aspergillus calidoustus, Aspergillus ochraceoroseus, Aspergillus nomius, Aspergillus aculeatus, Aspergillus carbonarius, Aspergillus parasiticus, Aspergillus kawachii, Aspergillus luchuensis, Aspergillus brasiliensis, Aspergillus foetidus, Aspergillus sojae, Aspergillus fumigatus, Aspergillus oryzae, Aspergillus nidulans, Aspergillus japonicus.

16. A method of producing a mutant filamentous fungal host cell according to claim 1 comprising:
a) providing a parent filamentous fungal host cell;
b) modifying the parent filamentous fungal host cell to yield a mutant filamentous fungal host cell which, if compared with a parent filamentous fungal host cell which has not been modified, when measured under the same conditions, is deficient in the cell in a polypeptide comprising a polypeptide selected from:
i. a polypeptide with an amino acid sequence according to SEQ ID NO: 5 or a polypeptide with an amino acid sequence at least 70% identical to the amino acid sequence according to SEQ ID NO: 5 which is a functional equivalent of the polypeptide with the amino acid sequence according to SEQ ID NO: 5;
ii. a polypeptide with an amino acid sequence according to SEQ ID NO: 6 or a polypeptide with an amino acid sequence at least 70% identical to the amino acid sequence according to SEQ ID NO: 6 which is a functional equivalent of the polypeptide with the amino acid sequence according to SEQ ID NO: 6;
iii. a polypeptide with an amino acid sequence according to SEQ ID NO: 4 or a polypeptide with an amino acid sequence at least 70% identical to the amino acid sequence according to SEQ ID NO: 4 which is a functional equivalent of the polypeptide with the amino acid sequence according to SEQ ID NO: 4;
wherein the mutant filamentous fungal host cell produces extracellularly less oxalic acid than the parent filamentous fungal host cell which has not been modified, when cultured and measured under the same conditions.

17. The method according to claim 16 wherein the mutant obtained in b) is said mutant.

18. A method for production of a compound of interest by microbial fermentation comprising:
a) providing a mutant filamentous fungal host cell according to claim 1, capable of expressing the compound of interest,
b) culturing said mutant filamentous fungal host cell under conditions conducive to the expression of the compound of interest,
c) optionally isolating the compound of interest from the culture medium.

19. The method according to claim 18 wherein the compound of interest is a biological compound selected from the group consisting of biomass, a biopolymer, a metabolite, optionally the compound of interest is selected from a biopolymer or a primary or secondary metabolite.


SEQUENCE LISTING (49 sequences)

SEQ ID NO: 1 - protein, 526 aa, Rasamsonia emersonii
  misc_feature: Protein sequence of proline specific endoprotease BC2G079 (previously described in WO2015177171, example 1, as SEQ ID NO 2)
MPSLSSLVALTASLVSLAAAAAPRLPLPPRPPLPPRDPLHGPTNASATFQQLIDHNHPELGTFSQRYWWNDEFWKGPGSPVVLFTPGEEDASGYVGYLKNTTITGLIAQTIGGAVIVLEHRYWGQSSPYDSLTTKNLQYLTLKQSIADLTYFAKTVKLPFDRNGSSNADKAPWVLSGGSYSGALSAWTASTSPGTFWAYHASSAPVEAIYDYWQYFAPVQDGLPANCSKDLSRVVDYIDSVLQSGNATAKQQLKDLFGLGALEHDDDFASALENGPWLWQSNSFYDPYPPVYEFCDYVENAYASPPVAAGPDGVGLEKALSGYATWWNKVFFPGYCATYGYWSSNDSIACFDTYNQSSPMFTDLSVSNTINRQWNWFLCNEPFFYWQDGAPKNVPSIVSRLVTAEYWQRQCPLFFPEEDGYTYGSAKGKTAADVNAWTKGWFLTNTTRLIWTNGELDPWRSAGVSSKFRPGGPLQSTPQAPLQLIPEGVHCYDLILKNAEANAGVQRVVTNEVAQIKAWVNEYYRK

SEQ ID NO: 2 - DNA, 4213 bp, Aspergillus niger
  misc_feature: Genomic sequence of the An14g05670 (oreR) gene
cccccctcccccccccctcttgttgtgcaaacactgtgcatagaagaggcctcctgatacgccgagctgaccaacgacaggtgtgaaacgatggtcggtctaacagatgcctagagaattagggtgtggcgaggtatacgggatgggggacatgccaaccgtcagacagccagatgggatccgggtgggagcgctaagtccgttaataagcggaatggcaatcagggttggtggccgtggaattatgcccactggcaaaaggacgtgtggctttctgcggttacatccgaataatgttgaaaaaaaaaaataaaaataaaaataaaaataaaaataaaaaaataaatccgctgcaatgtgatgatcaatccgcactagctgacgaccattgcggttccatgactgggcggattatattattttatttatcttgcctaacgcggatggcttggacttggtcgatcgccgcctggcgtggcgttgcgttctggattctcaaacgggagatcgaatccaagacattcatcctatcgagggactatggagccgagaaggaggcacgcgctccgtggttggtggagcagcctccgcgggctgaaaaaatatcagggatgcagggcaacttcagattaagccatgcaggagctcatacagccccccatttgtcgagcaattatctcagcaggaactagagttaccttcgttaggccgggtgaatctgattaaatccatcccatgctaatattacgcagactaagctacaagctacggtttcgggggggttccgcagtctgcaggccgaacgggggatattcacccccctcgagtcgggtcagatgtaccaaaacaaaaggatcaggatccttccatttgcttcccgatcggtcgatagggcagcttgagcgagacttgactaatctggcctattgcttcccctcaacattcgccccggatccgagatgggtggcccggaggttgtacttatcgacggcgtcccctcggtgcctttccctcttctctgatcctactattcatactgcctcaatacagctatatcttatacagcttcctcccaaacgccactcatttccattctgcttcttctctgattcatcctcgaacaactgagctgcttgctttcactgagtgcggctagtcaagctatgcccgctatgaccattacactgagtgaccagacacatactgtgtctgagacctcatcgacgaacaatggcatctcagggacagaatccaagaagtcaggcactcggaatggccatccccagagcaactgccaccagctgccgcctcccccatcgcaacccactgcggagggggaataccggtgcggaatctgcaacaagacatacagtcgacgtaggctactcttttctttatggttgcacagtgttgacacattttctccatgactactggctcccttgatcattctcagagagacggccacttaatgcacacatgtgtggttaacaagataatagttgatctgcgggaccggcaccgtcgccgttgtattaagaacattggccaggaacgccagtccaagcgcaaatcctgcgacgcctgtgcgcagaagaagctgcggtgttccatgacccggccctcctgtagccgatgtatccaaagccgacggccttgcgtgtatccgcagtcttctattcctgttcaagctcccaatcttgatgatgctcaagataatattgttacgtcttccggctcccttcactcagtttcggtctgcgccggtataatacctggtggcacgacatgggcccttcccacatatccgttcgacaccccgtctactgccgacgaagtaaatgatgtttcgggctcctcctggagtcccgaaactccaacaaatgccgaacttgcgttccaaaccgttaacgagagtactatgtttcccatgcaagatgtctcgttgatgaactccccgccctggcaagaggatctccatgaacggcccgaaacccttgggctggaggagttcttttccgggtcgcttgacagttgttcgccagctatgtatataccaccgacgatggggtcctctccagtgaccatggtacctcctaccccggctactctcccgggcgactcttatgctacgggaggcggactgagtatgtcatcggtatcccctggctatttcgataatttctggcccgataacatggcgtccaccgatgaagaccacgaaacttggcgattcggcacgtacacagatagccggtccggatcggggttcggccaaaacttcctgcccaaaacaagtcatcatagcccgggtgacatcggtgatctctatcaagagctgttcagcttgttacgtgagtaccccgggctggcactgcagagacagttctactcgcctttcctacaccacgagctacaagggtatgcgatgcaatcgttgggccagccaattagcactactctgagttcgatatcatcctatgctagttatctggaggcttgtgatacatttgatttgtcaatgcacgatggggagcggctcgcagcagcagaaggctgcgtggtagcgcttcatgctgtctgtgttcaacagatcctgagcattttcggcgataacttcgccaccaccggcttcagcaagctcccaaactttgcaggcgatcaagggcataccgagactcctgttgacattctcttgagggtatgtttccccccactccgattgccctacgccccaccctatgatttgagctgacctgagtggccctagatgacgcagcgggtctacaaacttcatgaggacgttcttcgcaccccccatgaagatgaaacagattggcgccgatggaaattcgccgaatccctgcgacgtaatattttcttcgccaatataatccgaacattagcaggacgggcccgccgttacaacgggatcaacattgacccgctagatagcgcgatccttcttcagctgccgttgccggcgccggaggaaatgtggcgagcacgcagcgagggggagtggatgatagcccgcgcacagacgcagcggtcgtgggcgcggggcgtggatggcagcatgctgccagccctgcggaccctacaacagctgctggtgctggaagaggttggcagtgtaagtgttacgttattgcttcccatcacacgcatgattctggcgtgtgccaagctcaatggagcttcatatggctgaggtggaccggtgggccccgcgatagggcgattatgatcgtcacggatgaatttctttttgcatgattttcctgtttccaaaataccctgttcttctcacgaagactacgatcatcgtgggatgtattagttttgtatccgctagagagtagagactagatcctgattgcaaggctttatgattatgattctcagtgttgtttcttgttctgttgttgaacccgtcccgaaatcggaacccaattccgtgggggatctcgacattgatagatcgcatcacgtctttagccgtcgccggattttgatgatagccccattgtggccaaactaacggtgactgcagagcccgtgccgacatgcacggatcaaaaagggatagattgaaatcagccaaacaaaaactgttgggcgcatgtatgagtgagaactggacctggggctgggcttggctggctgagacgccaaaatccggacacacggcgatgccgaggcctcgactcacgacgaacatgcaggccgtcaggcaggcaggcaggcaggcagacaggctggaagacgaggaatcgcgaccggacggcgaacagggtccaggcaggttagtgaggataggttcagcgagccgatacggtacctcgttagtgctccttagttctgaccagcttgctagccttcccgcggatggtaacctccggcgggggtccggcatccacccgtgggatttacttggggggtttttttgtacctctcgtgtcgatcgcagctatctcctccgaggttcgtggccaatcgtttgtatgcaggagatgcagtagtgtcgacgggctaggacgagttggttaacatctcctgggtggtggattggtctcgccgagtagtaagtagtatctagtagtagtaagtatctttctagtactagccgttgttttcatactaacgagttgttggcgatgaggataggagtgggggaggggagagatattatcccactgtgtgctatttt

SEQ ID NO: 3 - DNA, 1857 bp, Aspergillus niger
  misc_feature: cDNA of the An14g05670 (oreR) gene
atgcccgctatgaccattacactgagtgaccagacacatactgtgtctgagacctcatcgacgaacaatggcatctcagggacagaatccaagaagtcaggcactcggaatggccatccccagagcaactgccaccagctgccgcctcccccatcgcaacccactgcggagggggaataccggtgcggaatctgcaacaagacatacagtcgacttgatctgcgggaccggcaccgtcgccgttgtattaagaacattggccaggaacgccagtccaagcgcaaatcctgcgacgcctgtgcgcagaagaagctgcggtgttccatgacccggccctcctgtagccgatgtatccaaagccgacggccttgcgtgtatccgcagtcttctattcctgttcaagctcccaatcttgatgatgctcaagataatattgttacgtcttccggctcccttcactcagtttcggtctgcgccggtataatacctggtggcacgacatgggcccttcccacatatccgttcgacaccccgtctactgccgacgaagtaaatgatgtttcgggctcctcctggagtcccgaaactccaacaaatgccgaacttgcgttccaaaccgttaacgagagtactatgtttcccatgcaagatgtctcgttgatgaactccccgccctggcaagaggatctccatgaacggcccgaaacccttgggctggaggagttcttttccgggtcgcttgacagttgttcgccagctatgtatataccaccgacgatggggtcctctccagtgaccatggtacctcctaccccggctactctcccgggcgactcttatgctacgggaggcggactgagtatgtcatcggtatcccctggctatttcgataatttctggcccgataacatggcgtccaccgatgaagaccacgaaacttggcgattcggcacgtacacagatagccggtccggatcggggttcggccaaaacttcctgcccaaaacaagtcatcatagcccgggtgacatcggtgatctctatcaagagctgttcagcttgttacgtgagtaccccgggctggcactgcagagacagttctactcgcctttcctacaccacgagctacaagggtatgcgatgcaatcgttgggccagccaattagcactactctgagttcgatatcatcctatgctagttatctggaggcttgtgatacatttgatttgtcaatgcacgatggggagcggctcgcagcagcagaaggctgcgtggtagcgcttcatgctgtctgtgttcaacagatcctgagcattttcggcgataacttcgccaccaccggcttcagcaagctcccaaactttgcaggcgatcaagggcataccgagactcctgttgacattctcttgaggatgacgcagcgggtctacaaacttcatgaggacgttcttcgcaccccccatgaagatgaaacagattggcgccgatggaaattcgccgaatccctgcgacgtaatattttcttcgccaatataatccgaacattagcaggacgggcccgccgttacaacgggatcaacattgacccgctagatagcgcgatccttcttcagctgccgttgccggcgccggaggaaatgtggcgagcacgcagcgagggggagtggatgatagcccgcgcacagacgcagcggtcgtgggcgcggggcgtggatggcagcatgctgccagccctgcggaccctacaacagctgctggtgctggaagaggttggcagtgtaagtgttacgttattgcttcccatcacacgcatgattctggcgtgtgccaagctcaatggagcttcatatggctga

SEQ ID NO: 4 - protein, 618 aa, Aspergillus niger
  misc_feature: Protein sequence of OreR oxalate C6 transcriptional regulator from Aspergillus niger
MPAMTITLSDQTHTVSETSSTNNGISGTESKKSGTRNGHPQSNCHQLPPPPSQPTAEGEYRCGICNKTYSRLDLRDRHRRRCIKNIGQERQSKRKSCDACAQKKLRCSMTRPSCSRCIQSRRPCVYPQSSIPVQAPNLDDAQDNIVTSSGSLHSVSVCAGIIPGGTTWALPTYPFDTPSTADEVNDVSGSSWSPETPTNAELAFQTVNESTMFPMQDVSLMNSPPWQEDLHERPETLGLEEFFSGSLDSCSPAMYIPPTMGSSPVTMVPPTPATLPGDSYATGGGLSMSSVSPGYFDNFWPDNMASTDEDHETWRFGTYTDSRSGSGFGQNFLPKTSHHSPGDIGDLYQELFSLLREYPGLALQRQFYSPFLHHELQGYAMQSLGQPISTTLSSISSYASYLEACDTFDLSMHDGERLAAAEGCVVALHAVCVQQILSIFGDNFATTGFSKLPNFAGDQGHTETPVDILLRMTQRVYKLHEDVLRTPHEDETDWRRWKFAESLRRNIFFANIIRTLAGRARRYNGINIDPLDSAILLQLPLPAPEEMWRARSEGEWMIARAQTQRSWARGVDGSMLPALRTLQQLLVLEEVGSVSVTLLLPITRMILACAKLNGASYG

SEQ ID NO: 5 - protein, 55 aa, artificial
  Consensus sequence of DNA binding domain as deduced in Figure 5
  misc_feature (11)..(19): Xaa can be any naturally occurring amino acid
  misc_feature (22)..(22): Xaa can be any naturally occurring amino acid
  misc_feature (26)..(27): Xaa can be any naturally occurring amino acid
  misc_feature (33)..(33): Xaa can be any naturally occurring amino acid
  misc_feature (36)..(38): Xaa can be any naturally occurring amino acid
  misc_feature (40)..(41): Xaa can be any naturally occurring amino acid
  misc_feature (43)..(43): Xaa can be any naturally occurring amino acid
  misc_feature (46)..(46): Xaa can be any naturally occurring amino acid
  misc_feature (48)..(51): Xaa can be any naturally occurring amino acid
  misc_feature (53)..(53): Xaa can be any naturally occurring amino acid
DLRDRHRRRCXXXXXXXXXSKXKSCXXCAQKKXRCXXXRXXCXRCXQXXXXCXYP

SEQ ID NO: 6 - protein, 55 aa, Aspergillus niger
  misc_feature: Putative DNA binding domain of XP_001401190.2
DLRDRHRRRCIKNIGQERQSKRKSCDACAQKKLRCSMTRPSCSRCIQSRRPCVYP

SEQ ID NO: 7 - protein, 55 aa, Aspergillus luchuensis
  misc_feature: Putative DNA binding domain of GAT19234.1
DLRDRHRRRCINNIGQERQSKRKSCDACAQKKLRCSMTRSSCSRCLQSRRPCVYP

SEQ ID NO: 8 - protein, 55 aa, Aspergillus parasiticus SU-1
  misc_feature: Putative DNA binding domain of KJK66719.1
DLRDRHRRRCIKTAGQERLSKRKSCEICAQKKLRCSMTRPACARCVQMGTACHYP

SEQ ID NO: 9 - protein, 55 aa, Aspergillus ochraceoroseus
  misc_feature: Putative DNA binding domain of KKK23905.1
DLRDRHRRRCIKTIGQERQSKRKSCETCAQKKLRCSMTRPACSRCIQIRTPCRYP

SEQ ID NO: 10 - protein, 55 aa, Aspergillus bombycis
  misc_feature: Putative DNA binding domain of OGM46820.1
DLRDRHRRRCIKTAGQERLSKRKSCETCAQKKLRCSMTRPACARCIQMGTACHYP

SEQ ID NO: 11 - protein, 55 aa, Aspergillus tubingensis CBS 134.48
  misc_feature: putative DNA binding domain of OJI89400.1
DLRDRHRRRCIKNIGQERQSKRKSCDACAQKKLRCSMTRPSCSRCLQSRRPCVYP

SEQ ID NO: 12 - protein, 55 aa, Aspergillus brasiliensis CBS 101740; identical to SEQ ID NO: 11
  misc_feature: Putative DNA binding domain of OJJ77274.1

SEQ ID NO: 13 - protein, 55 aa, Aspergillus luchuensis CBS 106.47; identical to SEQ ID NO: 11
  misc_feature: Putative DNA binding domain of OJZ84680.1

SEQ ID NO: 14 - protein, 55 aa, Aspergillus carbonarius ITEM 5010; identical to SEQ ID NO: 11
  misc_feature: Putative DNA binding domain of OOF93142.1

SEQ ID NO: 15 - protein, 55 aa, Aspergillus oryzae RIB40; identical to SEQ ID NO: 8
  misc_feature: Putative DNA binding domain of XP_001818758.2

SEQ ID NO: 16 - protein, 55 aa, Aspergillus flavus NRRL3357; identical to SEQ ID NO: 8
  misc_feature: Putative DNA binding domain of XP_002380179.1

SEQ ID NO: 17 - protein, 55 aa, Aspergillus nomius NRRL 13137
  misc_feature: Putative DNA binding domain of XP_015405413.1
DLRDRHRRRCIKTAGQERLSKRKSCETCAQKKLRCSMTRPACARCVQMGTACHYP

SEQ ID NO: 18 - protein, 55 aa, Penicillium camemberti
  misc_feature: Putative DNA binding domain of CRL26276.1
DLRDRHRRRCIAKGGQARKSKQKSCETCAQKKIRCSLARPNCARCAQLGISCQYP

SEQ ID NO: 19 - protein, 55 aa, Aspergillus niger; identical to SEQ ID NO: 6
  misc_feature: Putative DNA binding domain of CAK42128.1

SEQ ID NO: 20 - protein, 55 aa, Penicillium brasilianum
  misc_feature: Putative DNA binding domain of CEJ54143.1
DLRDRHRRRCAKTFGQERASKRKSCETCAQKKLRCSLTRPACSRCIQMGSACHYP

SEQ ID NO: 21 - protein, 55 aa, Penicillium rubens Wisconsin 54-1255
  misc_feature: Putative DNA binding domain of XP_002563980.1
DLRDRHRRRCAAKGGQSGKSKQKSCETCAQKKIRCSLSRPNCARCAQLGISCHYP

SEQ ID NO: 22 - protein, 55 aa, Aspergillus aculeatus ATCC 16872; identical to SEQ ID NO: 11
  misc_feature: Putative DNA binding domain of XP_020057899.1

SEQ ID NO: 23 - protein, 55 aa, Aspergillus calidoustus
  misc_feature: Putative DNA binding domain of CEL10827.1
DLRDRHRRRCIKSIGQERLSKRKSCETCAQKKLRCSMTRPACTRCLQIGIHCQYP

SEQ ID NO: 24 - protein, 55 aa, Arthroderma otae CBS 113480
  misc_feature: Putative DNA binding domain of XP_002850264.1
DLRDRHRRRCIKSANHERVSKRKSCETCAQKKLRCCLTRPACSRCVQTQTPCHYP

SEQ ID NO: 25 - protein, 556 aa, Aspergillus luchuensis
  misc_feature: Protein sequence of GAT19234.1
MQRLVVNQKIGDLRDRHRRRCINNIGQERQSKRKSCDACAQKKLRCSMTRSSCSRCLQSRRPCVYPQSSVPVQTPSLDDPQDNIASSAGSIHSVPICAGIIPGGTTWALPTYPFDTPSTADELHDVSGSSWSPETPTNAELAFHAVNESTMLPMQDASLMNSPPWQDDLHEQSEAFGLEEFFPGSLDSCSHAMYMPPTMRSSPVTMVPPTPATLPGDSYAAGGGLNMSSVSPGYFDNIWPDMASNDEENETWRFSTYTHSLTGSGFGQSFLPKTSHHSPGDVGDLYQELFSLLREYPGLALQRQFYSPFLHHELQGYAMQSMGQPLSTTLSSISSYANYLETCDTFDLSMHNGERLAAAEGCVVALHAVCVQQILSLFGDNFATNGLSKLPSLTGEQGHPETPVDILLRMTQRVYKLHEDILRTPHEDETDWRRWKFAESLRRNIFFANIIRTLAGRARRYNGINIDPLDSAILLQLPLPAPEEMWRARSEGEWMIARAQTQRSWARGVDGSMLPALRTLQQLLVLEEVGSVSVTLLLPITRMILACAKLNGASYG

SEQ ID NO: 26 - protein, 617 aa, Aspergillus tubingensis CBS 134.48
  misc_feature: Protein sequence of OJI89400.1
MPAMTITLSDQTHTMSEASPKKDGIAGTEVKKPGTRHGHSQSNCHHLPPPPSQPTAEGEYRCGICNKTYSRRDLRDRHRRRCIKNIGQERQSKRKSCDACAQKKLRCSMTRPSCSRCLQSRRPCVYPQSSVPVQTPSLDDPQDNIASSAGSIHSVPICAGIIPGGTTWALPTYPFDTPSTADELHDISGSSWSPETPTNAELAFHAVNESTMLPMQDASLMNSPPWQDDLHEQSETFGLEEFFPGSLDSCSHAMYMPPTMRSSPVTMVPPTPATLSGDSYAAGGGLNMSSVSPGYFDNIWPDMASNDEENETWRFSTYTHSRTGSGFGQSFLPKTSHHSPDDVGDLYQELFSLLREYPGLALQRQFYSPFLHHELQGYAMQSMGQPLSTTLSSISSYANYLETCDTFDLSMHNGERLAAAEGCVVALHAVCVQQILSLFGDNFATNGLSKLPSLTGDQGHPETPVDILLRMTQRVYKLHEDILRTPHEDETDWRRWKFAESLRRNIFFANIIRTLAGRARRYNGINIDPLDSAILLQLPLPAPEEMWRARSEGEWMIARAQTQRSWARGVDGSMLPALRTLQQLLVLEEVGSVSVTLLLPITRMILACAKLNGASYG

SEQ ID NO: 27 - protein, 618 aa, Aspergillus brasiliensis CBS 101740
  misc_feature: Protein sequence of OJJ77274.1
MPAMTITLSDQTHTMSEASSKRDGISRAESRKPGARHGHSQSTCHQLPPPPSQPTAEGEYRCGICNKTYSRRDLRDRHRRRCIKNIGQERQSKRKSCDACAQKKLRCSMTRPSCSRCLQSRRPCVYPQSSVPVQASSLDDSQDNLVSSVGSIHAVPVCAGIIPGGTTWALPTYPFDTPSTGDELNDVPGASWSPETPTNAELAIQTVNDSTMFPMQDVSLMNSPTWQDDLHEQPETYGLEEFFPGSLDSCSQTMYMPPTMGSSPVTMVPPTPATLPGDSYATGGGLNMRSVSAGYFDSFWPDNLVSNDEDNETWRLGTYTDSRTGSGFGQSFLPKTHHHSPGDIGDLYQELFSLLREYPGLALQRQFYSPFLHHELQGYAMQSMGQPLSTTLSSISSYATYLETCDTFDLSMHNGERLAAAEGCVVALHAVCVHQILSIFGDNFATASFSKLPSFAGDHGNSEAPVEILLRMTQRVYKLHEDILRTPHEDETDWRRWKFAETLRRNIFFANIVRTLAGRARRYNGINIDPLDSAILLQLPLPAPEEMWRARSEGEWMIARAQTQQSWARGVDGSMLPALRTLQQLLVLEEVGSVSVTLLLPITRMILACAKLNGASYG

SEQ ID NO: 28 - protein, 617 aa, Aspergillus luchuensis CBS 106.47
  misc_feature: Protein sequence of OJZ84680.1
MPAVTITLSDQTHTMSEASPKRDGIARTEVKKPGTRHGHSQSNCHHLPPPPSQPTAEGEYRCGICNKTYSRRDLRDRHRRRCIKNIGQERQSKRKSCDACAQKKLRCSMTRPSCSRCLQSRRPCVYPQSSVPVQTPSLDDPQDNIASSAGSIHSVPICAGIIPGGTTWALPTYPFDTPSTADELHDVSGSSWSPETPTNAELAFHAVNESTMLPMQDASLMNSPPWQDDLHEQSEAFGLEEFFPGSLDSCSHAMYMPPTMRSSPVTMVPPTPATLPGDSYAAGGGLNMSSVSPGYFDNIWPDMASNDEENETWRFSTYTHSLTGSGFGQSFLPKTSHHSPGDVGDLYQELFSLLREYPGLALQRQFYSPFLHHELQGYAMQSMGQPLSTTLSSISSYANYLETCDTFDLSMHNGERLAAAEGCVVALHAVCVQQILSLFGDNFATNGLSKLPSLTGEQGHPETPVDILLRMTQRVYKLHEDILRTPHEDETDWRRWKFAESLRRNIFFANIIRTLAGRARRYNGINIDPLDSAILLQLPLPAPEEMWRARSEGEWMIARAQTQRSWARGVNGSMLPALRTLQQLLVLEEVGSVSVTLLLPITRMILACAKLNGASYG

SEQ ID NO: 29 - protein, 557 aa, Aspergillus niger
  misc_feature: Protein sequence of CAK42128.1
MHTCVVNKIIVDLRDRHRRRCIKNIGQERQSKRKSCDACAQKKLRCSMTRPSCSRCIQSRRPCVYPQSSIPVQAPNLDDAQDNIVTSSGSLHSVSVCAGIIPGGTTWALPTYPFDTPSTADEVNDVSGSSWSPETPTNAELAFQTVNESTMFPMQDVSLMNSPPWQEDLHERPETLGLEEFFSGSLDSCSPAMYIPPTMGSSPVTMVPPTPATLPGDSYATGGGLSMSSVSPGYFDNFWPDNMASTDEDHETWRFGTYTDSRSGSGFGQNFLPKTSHHSPGDIGDLYQELFSLLREYPGLALQRQFYSPFLHHELQGYAMQSLGQPISTTLSSISSYASYLEACDTFDLSMHDGERLAAAEGCVVALHAVCVQQILSIFGDNFATTGFSKLPNFAGDQGHTETPVDILLRMTQRVYKLHEDVLRTPHEDETDWRRWKFAESLRRNIFFANIIRTLAGRARRYNGINIDPLDSAILLQLPLPAPEEMWRARSEGEWMIARAQTQRSWARGVDGSMLPALRTLQQLLVLEEVGSVSVTLLLPITRMILACAKLNGASYG

SEQ ID NO: 30 - DNA, 2125 bp, Aspergillus niger
tccgctgcaatgtgatgatcaatccgcactagctgacgaccattgcggttccatgactgggcggattatattattttatttatcttgcctaacgcggatggcttggacttggtcgatcgccgcctggcgtggcgttgcgttctggattctcaaacgggagatcgaatccaagacattcatcctatcgagggactatggagccgagaaggaggcacgcgctccgtggttggtggagcagcctccgcgggctgaaaaaatatcagggatgcagggcaacttcagattaagccatgcaggagctcatacagccccccatttgtcgagcaattatctcagcaggaactagagttaccttcgttaggccgggtgaatctgattaaatccatcccatgctaatattacgcagactaagctacaagctacggtttcgggggggttccgcagtctgcaggccgaacgggggatattcacccccctcgagtcgggtcagatgtaccaaaacaaaaggatcaggatccttccatttgcttcccgatcggtcgatagggcagcttgagcgagacttgactaatctggcctattgcttcccctcaacattcgccccggatccgagatgggtggcccggaggttgtacttatcgacggcgtcccctcggtgcctttccctcttctctgatcctactattcatactgcctcaatacagctatatcttatacagcttcctcccaaacgccactcatttccattctgcttcttctctgattcatcctcgaacaactgagctgcttgctttcactgagtgcggctagtcaagctatgcccgctatgaccattacactgagtgaccagacacatactgtgtctgagacctcatcgacgaacaatggcatctcagggacagaatccaagaagtcaggcactcggaatggccatccccagagcaactgccaccagctgccgcctcccccatcgcaacccactgcggagggggaataccggtgcggaatctgcaacaagacatacagtcgacgtaggctactcttttctttatggttgcacagtgttgacacattttctccatgactactggctcccttgatcattctcagagagacggccacttaaggtggaccggtgggccccgcgatagggcgattatgatcgtcacggatgaatttctttttgcatgattttcctgtttccaaaataccctgttcttctcacgaagactacgatcatcgtgggatgtattagttttgtatccgctagagagtagagactagatcctgattgcaaggctttatgattatgattctcagtgttgtttcttgttctgttgttgaacccgtcccgaaatcggaacccaattccgtgggggatctcgacattgatagatcgcatcacgtctttagccgtcgccggattttgatgatagccccattgtggccaaactaacggtgactgcagagcccgtgccgacatgcacggatcaaaaagggatagattgaaatcagccaaacaaaaactgttgggcgcatgtatgagtgagaactggacctggggctgggcttggctggctgagacgccaaaatccggacacacggcgatgccgaggcctcgactcacgacgaacatgcaggccgtcaggcaggcaggcaggcaggcagacaggctggaagacgaggaatcgcgaccggacggcgaacagggtccaggcaggttagtgaggataggttcagcgagccgatacggtacctcgttagtgctccttagttctgaccagcttgctagccttcccgcggatggtaacctccggcgggggtccggcatccacccgtgggatttacttggggggtttttttgtacctctcgtgtcgatcgcagctatctcctccgaggttcgtggccaatcgtttgtatgcaggagatgcagtagtgtcgacgggctaggacgagttggttaacatctcctgggtggtggattggtctcgccgagtagtaagtagtatctagtagtagtaagtatctttctagtactagccgttgttttcatactaacgagttgttggcgatgaggataggagtgggggaggggagagatattatcccactgtgtgctatttt

SEQ ID NO: 31 - protein, 510 aa, Aspergillus niger
MTRPSCSRCIQSRRPCVYPQSSIPVQAPNLDDAQDNIVTSSGSLHSVSVCAGIIPGGTTWALPTYPFDTPSTADEVNDVSGSSWSPETPTNAELAFQTVNESTMFPMQDVSLMNSPPWQEDLHERPETLGLEEFFSGSLDSCSPAMYIPPTMGSSPVTMVPPTPATLPGDSYATGGGLSMSSVSPGYFDNFWPDNMASTDEDHETWRFGTYTDSRSGSGFGQNFLPKTSHHSPGDIGDLYQELFSLLREYPGLALQRQFYSPFLHHELQGYAMQSLGQPISTILSSISSYASYLEACDTFDLSMHDGERLAAAEGCVVALHAVCVQQILSIFGDNFATTGFSKLPNFAGDQGHTETPVDILLRMTQRVYKLHEDVLRTPHEDETDWRRWKFAESLRRNIFFANIIRTLAGRARRYNGINIDPLDSAILLQLPLPAPEEMWRARSEGEWMIARAQTQRSWARGVDGSMLPALRTLQQLLVLEEVGSVSVTLLLPITRMILACAKLNGASYG

SEQ ID NO: 32 - protein, 70 aa, Aspergillus niger
GEYRCGICNKTYSRLDLRDRHRRRCIKNIGQERQSKRKSCDACAQKKLRCSMTRPSCSRCIQSRRPCVYP

SEQ ID NO: 33 - protein, 70 aa, Aspergillus parasiticus
GDFRCGICNKTYSRRDLRDRHRRRCIKTAGQERLSKRKSCEICAQKKLRCSMTRPACARCVQMGTACHYP

SEQ ID NO: 34 - protein, 70 aa, Aspergillus ochraceoroseus
GEFRCGICNKTYSRRDLRDRHRRRCIKTIGQERQSKRKSCETCAQKKLRCSMTRPACSRCIQIRTPCRYP

SEQ ID NO: 35 - protein, 70 aa, Aspergillus bombycis
GDFRCGICNKTYSRRDLRDRHRRRCIKTAGQERLSKRKSCETCAQKKLRCSMTRPACARCIQMGTACHYP

SEQ ID NO: 36 - protein, 70 aa, Aspergillus tubingensis
GEYRCGICNKTYSRRDLRDRHRRRCIKNIGQERQSKRKSCDACAQKKLRCSMTRPSCSRCLQSRRPCVYP

SEQ ID NO: 37 - protein, 70 aa, Aspergillus brasiliensis; identical to SEQ ID NO: 36

SEQ ID NO: 38 - protein, 70 aa, Aspergillus luchuensis; identical to SEQ ID NO: 36

SEQ ID NO: 39 - protein, 70 aa, Aspergillus carbonarius; identical to SEQ ID NO: 36

SEQ ID NO: 40 - protein, 70 aa, Aspergillus oryzae; identical to SEQ ID NO: 33

SEQ ID NO: 41 - protein, 70 aa, Aspergillus flavus; identical to SEQ ID NO: 33

SEQ ID NO: 42 - protein, 70 aa, Aspergillus nomius
GDFRCGICNKTYSRRDLRDRHRRRCIKTAGQERLSKRKSCETCAQKKLRCSMTRPACARCVQMGTACHYP

SEQ ID NO: 43 - protein, 70 aa, Penicillium camemberti
DEFRCEICHKTYSRHDLRDRHRRRCIAKGGQARKSKQKSCETCAQKKIRCSLARPNCARCAQLGISCQYP

SEQ ID NO: 44 - protein, 63 aa, Aspergillus niger
CVVNKIIVDLRDRHRRRCIKNIGQERQSKRKSCDACAQKKLRCSMTRPSCSRCIQSRRPCVYP

SEQ ID NO: 45 - protein, 69 aa, Penicillium brasilianum
EYRCGLCNKTYSRRDLRDRHRRRCAKTFGQERASKRKSCETCAQKKLRCSLTRPACSRCIQMGSACHYP

SEQ ID NO: 46 - protein, 67 aa, Penicillium rubens
RCEICNKVYSRRDLRDRHRRRCAAKGGQSGKSKQKSCETCAQKKIRCSLSRPNCARCAQLGISCHYP

SEQ ID NO: 47 - protein, 70 aa, Aspergillus aculeatus
GDFRCGICNKTYSRRDLRDRHRRRCIKNIGQERQSKRKSCDACAQKKLRCSMTRPSCSRCLQSRRPCVYP

SEQ ID NO: 48 - protein, 70 aa, Aspergillus calidoustus
GEFRCGICHKTYGRRDLRDRHRRRCIKSIGQERLSKRKSCETCAQKKLRCSMTRPACTRCLQIGIHCQYP

SEQ ID NO: 49 - protein, 70 aa, Arthroderma otae
GDFRCHHCNKSYRRRDLRDRHRRRCIKSANHERVSKRKSCETCAQKKLRCCLTRPACSRCVQTQTPCHYP